(19) United States
(12) Patent Application Publication
(10) Pub. No.: US 2026/0306490 A1
(43) Pub. Date: Oct. 8, 2026

(54) PIM1 INHIBITOR FOR TREATING MYELOPROLIFERATIVE NEOPLASMS

(71) Applicant: Sumitomo Pharma America, Inc., Marlborough, MA (US)

(72) Inventors: Emily M. Brooks, Scarborough, ME (US); Jason M. Foulks, Sandy, UT (US); Sujan Kabir, Acton, MA (US); Claudia A. Lebedinsky, Waltham, MA (US); Jian Mei, Chicago, IL (US); Mark L. Wade, Salt Lake City, UT (US); Clifford J. Whatcott, West Jordan, UT (US)

(21) Appl. No.: 19/125,538

(22) PCT Filed: Oct. 30, 2023
(86) PCT No.: PCT/US2023/078187

§ 371 Date: Apr. 29, 2025

Related U.S. Application Data
(60) Provisional application No. 63/381,711, filed on Oct. 31, 2022.
(60) Provisional application No. 63/425,522, filed on Nov. 15, 2022.
(60) Provisional application No. 63/501,221, filed on May 10, 2023.
(60) Provisional application No. 63/506,526, filed on Jun. 6, 2023.

(51) Int. Cl. A61K 31/5025; A61K 31/519; A61K 31/5377; A61P 35/00

(52) U.S. Cl. CPC A61K 31/5025; A61K 31/519; A61K 31/5377; A61P 35/00

(57) ABSTRACT

Methods for treatment of myeloproliferative neoplasms are provided, particularly in patients with poor bone marrow functioning, and difficult to treat human patients such as those with relapsed or refractory disease. The disclosed methods comprise administering Compound (1) having the following structure:

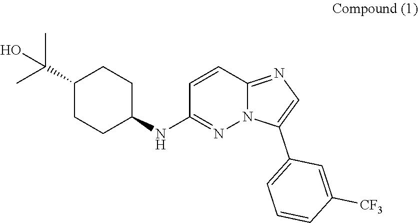 [SMILES: CC(C)(O)[C@H]1CC[C@H](Nc2ccc3ncc(-c4cccc(C(F)(F)F)c4)n3n2)CC1]

or a pharmaceutically acceptable salt thereof, and optionally a JAK kinase inhibitor and/or ACVR inhibitor, to a human subject in need thereof.

### RELATION TO PRIOR APPLICATIONS

The present application claims priority to U.S. Provisional Application Nos. 63/381,711 (filed Oct. 31, 2022), 63/425,522 (filed Nov. 15, 2022), 63/501,221 (filed May 10, 2023), and 63/506,526 filed Jun. 6, 2023 (collectively the “Prior Applications”). The contents of the Prior Applications are incorporated by reference as if fully set forth herein.

### TECHNICAL FIELD

The present technology relates to PIM kinase inhibitors, particularly selective PIM1 kinase inhibitors, for monotherapy and combination therapies for the treatment of myeloproliferative neoplasms such as myelofibrosis (MF).

### BACKGROUND

Despite meaningful advancements in the treatment of many hematological malignancies, significant unmet needs persist. Drug resistance, toxicities, poor response, and poor overall survival remain substantial challenges that require the development and evaluation of novel therapeutic agents.

Myelofibrosis (“MF”) is the most severe form of myeloproliferative neoplasms (“MPN”) characterized by bone marrow fibrosis, aberrant hematopoiesis, splenomegaly, symptoms deterioration, and frequent transformation to acute myeloid leukemia. The first line standard of care for MF is treatment with a JAK2 inhibitor such as ruxolitinib. However, some patients experience an inadequate response, their disease continues to progress, or they suffer adverse events, particularly thrombocytopenia and other loss of bone marrow function.

Numerous molecules have been developed to address these challenges, directed toward various biological targets. Many, such as navitoclax (an oral BCL-X_{I}/BCL-2 inhibitor), parsaclisib (an oral inhibitor of phosphatidylinositol 3-kinase delta (PI3Kδ)), and pelabresib (an oral bromodomain and extraterminal domain (BET) inhibitor (BETi)) have shown efficacy in animal models, only to fail or perform poorly when subsequently tested in rigorous human clinical trials, particularly in relapsed or refractory patients previously treated with JAK inhibitors, or when evaluating the total effect of the drug on the most common symptomatic burden reported by MPN human patients.

The PIM kinase family have been implicated in the regulation of cell growth, survival, and cell cycle and are overexpressed in various cancers. PIM1 kinase has been found to be significantly overexpressed in MF patients. TP-3654 is a highly selective oral PIM-1 kinase inhibitor.

There remains a need for treatment regimens in humans that exploit the activity of PIM kinase inhibitors, both selective PIM1 kinase inhibitors in general, and TP-3654 in particular, to treat hematological malignancies such as MF.

There also remains a need to treat the symptomatic burden of myeloproliferative neoplasms in humans, particularly those measured by the Total Symptoms Score.

A further need exists for augmenting existing MPN treatments without causing or exacerbating hematological toxicities.

An additional need exists in populations previously treated with JAK inhibitors who have relapsed disease, who have become refractory to treatment, are intolerant of JAK inhibitors, or a combination of two or more thereof.

A still further need exists in patients who suffer from poor bone marrow function as a consequence of prior drug treatment or the disease state itself.

### SUMMARY OF DISCLOSURE

This disclosure is based in part on the discovery that Compound (1) (TP-3654) demonstrates clinical activity in human myeloproliferative disorders (e.g., MF) with few or none of the hematological toxicities and poor bone marrow functioning associated with JAK inhibitors, as reported in the examples hereto. These discoveries allow for the treatment of patients with poor bone marrow function, for whom other treatments are often unavailable. They also allow for treatments with improved effect, given the prognostic role that poor bone marrow function plays in disease progression.

Thus in one aspect the disclosure provides a method for treating a myeloproliferative neoplasm in a human subject in need thereof, comprising administering to the subject a therapeutically effective amount of Compound (1) having the following structure:

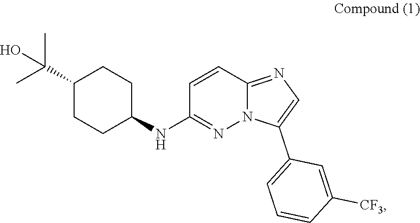 [SMILES: CC(C)(O)[C@H]1CC[C@H](Nc2ccc3ncc(-c4cccc(C(F)(F)F)c4)n3n2)CC1]

or a pharmaceutically acceptable salt thereof, wherein the subject has poor bone marrow functioning characterized by: thrombocytopenia (e.g. a platelet count of ≤150, 100, 75, 50, or 25×10⁹/L); anemia (e.g. a Hgb level≤10, 9, or 8 g/dL); transfusion dependence; or a combination thereof, and wherein the treatment induces a clinically meaningful response in the myeloproliferative neoplasm.

The disclosure is further based on the discovery that the lack of hematological toxicities observed with Compound (1) is also observed with other selective PIM1 inhibitors, but that these advantages are not observed with pan-PIM1 inhibitors, as shown in the examples hereto.

Thus, in another aspect the disclosure provides a method for treating a myeloproliferative neoplasm in a human subject in need thereof, comprising administering to the subject a therapeutically effective amount of a selective PIM1 inhibitor, wherein the subject has poor bone marrow functioning characterized by: thrombocytopenia (e.g. a platelet count of ≤150, 100, 75, 50, or 25×10⁹/L); anemia (e.g. a Hgb level≤10, 9, or 8 g/dL); transfusion dependence; or a combination thereof, wherein the treatment induces a clinically meaningful response in the myeloproliferative neoplasm.

The disclosure is further based on the discovery that Compound (1) is effective in human subjects who have previously failed JAK inhibitor therapy, and it is effective to a degree not seen in other treatments under development for myeloproliferative neoplasms, as shown by comparisons reported in the examples hereto.

Thus, in another aspect the disclosure provides a method of treating a myeloproliferative neoplasm in a human subject in need thereof, wherein the subject has previously been treated by a JAK inhibitor for the myeloproliferative neoplasm and optionally has relapsed disease or is refractory to the JAK inhibitor, comprising administering to the subject a therapeutically effective amount of Compound (1) having the following structure:

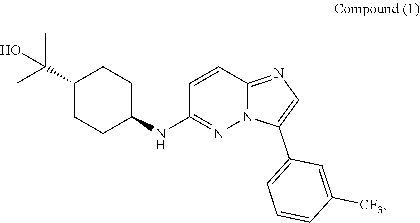 [SMILES: CC(C)(O)[C@H]1CC[C@H](Nc2ccc3ncc(-c4cccc(C(F)(F)F)c4)n3n2)CC1]

or a pharmaceutically acceptable salt thereof, wherein the treatment induces a clinically meaningful response in the myeloproliferative neoplasm.

The disclosure is also based on the discovery that Compound (1) is able to treat the symptomatic burden of myeloproliferative neoplasms, particularly as measured by the about 50% reductions in Total Symptoms Score (“TSS50”), when other treatments under development have failed.

Thus, in another aspect the disclosure provides a method of treating a myeloproliferative neoplasm by inducing TSS50 in a human subject in need thereof, comprising administering to the subject a therapeutically effective amount of Compound (1) having the following structure:

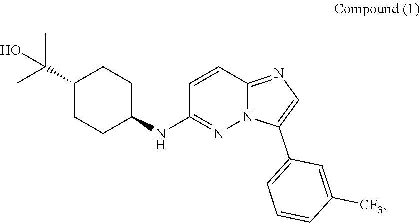 [SMILES: CC(C)(O)[C@H]1CC[C@H](Nc2ccc3ncc(-c4cccc(C(F)(F)F)c4)n3n2)CC1]

or a pharmaceutically acceptable salt thereof, wherein the myeloproliferative neoplasm is treated by inducing TSS50 in the subject, optionally at a time point between and including about 12 and 100 weeks (e.g., between and including about 20 and 75 weeks or between and including about 24 and 48 weeks).

The disclosure is also based on the discovery that there are minimal or no overlapping hematological toxicities when Compound (1) or another selective PIM1 inhibitor is administered in combination with other treatments for myeloproliferative neoplasms such as a JAK inhibitor and/or ACVR inhibitor (e.g., ruxolitinib and momelotinib), such as anemia, thrombocytopenia, transfusion dependence, or a combination of two or more thereof. At the same time, each of the molecules in the combination meaningfully contributes to the efficacy of the therapy, particularly in terms of TSS improvements and/or spleen volume reduction.

Thus, in another aspect the disclosure provides a method of treating a myeloproliferative neoplasm in a human subject in need thereof, comprising administering to the subject Compound (1) having the following structure:

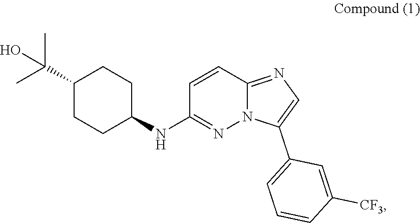 [SMILES: CC(C)(O)[C@H]1CC[C@H](Nc2ccc3ncc(-c4cccc(C(F)(F)F)c4)n3n2)CC1]

or a pharmaceutically acceptable salt thereof, and ruxolitinib or momelotinib or a pharmaceutically acceptable salt thereof in amounts that induce no overlapping hematological toxicities (e.g., anemia and/or thrombocytopenia and/or transfusion dependence), wherein the treatment induces a clinically meaningful response in the myeloproliferative neoplasm.

In another aspect the disclosure provides a method of treating a myeloproliferative neoplasm in a human subject in need thereof, comprising administering to the subject a selective PIM1 inhibitor and a JAK inhibitor and/or ACVR inhibitor (e.g., ruxolitinib or momelotinib) or a pharmaceutically acceptable salt thereof in amounts that induce no overlapping hematological toxicities (e.g., anemia and/or thrombocytopenia and/or transfusion dependence), wherein the treatment induces a clinically meaningful response in the myeloproliferative neoplasm.

The disclosure is still further based on the remarkable results observed from Compound (1) relative to other treatments for myeloproliferative neoplasms, in human subjects previously treated with a JAK inhibitor, and the enhanced probability of clinically meaningful improvements in SVR35 or TSS50.

Thus, in another aspect the disclosure provides a method of inducing a probability of SVR35 in a human subject affected by a myeloproliferative neoplasm about ≥15% or 20%, wherein the subject has previously been treated by a JAK inhibitor for the myeloproliferative neoplasm and optionally has relapsed disease or is refractory to the JAK inhibitor, comprising administering to the subject a therapeutically effective amount of Compound (1) having the following structure:

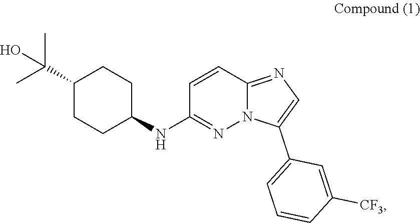 [SMILES: CC(C)(O)[C@H]1CC[C@H](Nc2ccc3ncc(-c4cccc(C(F)(F)F)c4)n3n2)CC1]

or a pharmaceutically acceptable salt thereof, wherein the treatment induces a probability of SVR35 in the subject≥15% or 20%.

In another aspect the disclosure provides a method of inducing a probability of TSS50 in a human subject affected by a myeloproliferative neoplasm about ≥30%, 35%, 40%, 45%, or 50%, wherein the subject has previously been treated by a JAK inhibitor for the myeloproliferative neoplasm and optionally has relapsed disease or is refractory to the JAK inhibitor, comprising administering to the subject a therapeutically effective amount of Compound (1) having the following structure:

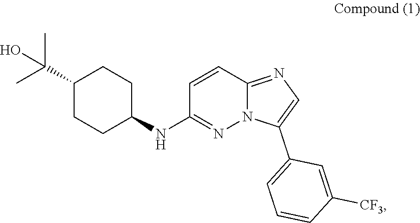 [SMILES: CC(C)(O)[C@H]1CC[C@H](Nc2ccc3ncc(-c4cccc(C(F)(F)F)c4)n3n2)CC1]

or a pharmaceutically acceptable salt thereof, wherein the treatment induces a probability of TSS50 in the subject about ≥30%, 35%, 40%, 45%, or 50%.

These and other aspects of embodiments of the disclosure will be apparent upon reference to the following detailed description. To this end, various references are set forth herein which describe in more detail certain background information, procedures, compounds and/or compositions, and are each hereby incorporated by reference in their entirety.

### BRIEF DESCRIPTION OF THE DRAWINGS

In the figures, identical reference numbers identify similar elements. The sizes and relative positions of elements in the figures are not necessarily drawn to scale and some of these elements are enlarged and positioned to improve figure legibility. Further, the particular shapes of the elements as drawn are not intended to convey any information regarding the actual shape of the particular elements and have been solely selected for ease of recognition in the figures.

FIG. 1A shows the in vitro effect of momelotinib, pacritinib, ruxolitinib, and the HCl salt of Compound (1) on human erythroid (CFU-E, BFU-E), according to the examples.

FIG. 1B shows the in vitro effect of momelotinib, pacritinib, ruxolitinib, and the HCl salt of Compound (1) on human megakaryocyte (CFU-MK), according to the examples.

FIG. 1C shows the in vitro effect of momelotinib, pacritinib, ruxolitinib, and the HCl salt of Compound (1) on human granulocyte-monocyte (CFU-GM), according to the examples.

FIG. 2A shows the in vitro effect of ruxolitinib, the HCl salt of Compound (1), and combinations thereof on human megakaryocyte (CFU-Mk), according to the examples.

FIG. 2B shows the in vitro effect of pacritinib, the HCl salt of Compound (1), and combinations thereof on human megakaryocyte (CFU-MK), according to the examples.

FIG. 2C shows the in vitro effect of momelotinib, the HCl salt of Compound (1), and combinations thereof on human megakaryocyte (CFU-MK), according to the examples.

FIG. 3A shows the in vitro effect of ruxolitinib, the HCl salt of Compound (1), and combinations thereof on human erythroid (CFU-E, BFU-E), according to the examples.

FIG. 3B shows the in vitro effect of pacritinib, the HCl salt of Compound (1), and combinations thereof on human erythroid (CFU-E, BFU-E), according to the examples.

FIG. 3C shows the in vitro effect of momelotinib, the HCl salt of Compound (1), and combinations thereof on human erythroid (CFU-E, BFU-E), according to the examples.

FIG. 4A shows the in vitro effect of ruxolitinib, the HCl salt of Compound (1), and combinations thereof on human granulocyte-monocyte (CFU-GM), according to the examples.

FIG. 4B shows the in vitro effect of pacritinib, the HCl salt of Compound (1), and combinations thereof on human granulocyte-monocyte (CFU-GM), according to the examples.

FIG. 4C shows the in vitro effect of momelotinib, the HCl salt of Compound (1), and combinations thereof on human granulocyte-monocyte (CFU-GM), according to the examples.

FIG. 5A shows patient platelet counts during monotherapy treatment with the HCl salt of Compound (1), according to the examples.

FIG. 5B shows patient hemoglobin during monotherapy treatment with the HCl salt of Compound (1), according to the examples.

FIG. 5C shows patient neutrophil during monotherapy treatment with the HCl salt of Compound (1), according to the examples.

FIG. 5D shows average patient blood counts (platelet, hemoglobin, and neutrophil) during monotherapy treatment with the HCl salt of Compound (1), according to the examples.

FIG. 5E shows best percent change of patient spleen volume reduction during monotherapy treatment with the HCl salt of Compound (1), according to the examples.

FIG. 5F shows best percent change of patient total symptom score reduction during monotherapy treatment with the HCl salt of Compound (1), according to the examples.

FIG. 5G shows best percent change of patient spleen volume reduction and total symptom score reduction during monotherapy treatment with the HCl salt of Compound (1), according to the examples.

FIG. 5H shows patient cytokine modulation, spleen volume reduction, and total symptom score reduction during monotherapy treatment with the HCl salt of Compound (1), according to the examples.

FIG. 5I shows patient cytokine modulation and total symptom score reduction at 4 weeks, 8 weeks, and 12 weeks of treatment with the HCl salt of Compound (1), according to the examples.

FIG. 5J shows patient cytokine modulation and total symptom score reduction at 12 weeks of treatment with the HCl salt of Compound (1), according to the examples.

FIG. 5K shows mean hemoglobin and platelet counts throughout 24-weeks of treatment for 22-evaluable patients treated with the HCl salt of Compound (1), according to the examples.

FIG. 5L shows patient cytokine modulation and total symptom score reduction at various time points of treatment with the HCl salt of Compound (1), according to the examples.

### DETAILED DESCRIPTION

In the following description, certain specific details are set forth in order to provide a thorough understanding of various embodiments of the disclosure. However, one skilled in the art will understand that embodiments of the disclosure may be practiced without these details.

### Definitions and Use of Terms

Unless the context requires otherwise, throughout the present specification and claims, the word “comprise” and variations thereof, such as “comprises” and “comprising,” are to be construed in an open, inclusive sense (i.e., as “including, but not limited to”).

Reference throughout this specification to “one embodiment” or “an embodiment” or “some embodiments” means that a particular feature, structure or characteristic described in connection with the embodiment is included in at least one embodiment of the present disclosure. Thus, the appearances of the phrases “in one embodiment” or “in an embodiment” or “in some embodiments” in various places throughout this specification are not necessarily all referring to the same embodiment. Furthermore, the particular features or characteristics may be combined in any suitable manner in one or more embodiments.

Myeloproliferative neoplasms (“MPN”) refer to a group of disorders in which bone marrow stem cells grow and reproduce abnormally. MPN abnormal stem cells produce excess numbers of one or more types of blood cells (e.g., red blood cells, white blood cells, and/or platelets). As disclosed herein, myeloproliferative neoplasms include, but are not limited to, polycythemia vera (PV), primary or essential thrombocythemia (ET), pre-MF, primary or idiopathic MF, secondary MF (e.g., MF secondary to polycythemia vera or essential thrombocythemia). In some embodiments, the MPN includes PV. In some embodiments, the MPN includes primary or essential thrombocythemia. In some embodiments, the MPN includes pre-MF. In some embodiments, the MPN includes primary or idiopathic MF. In some embodiments, the MPN includes secondary MF (e.g., MF secondary to polycythemia vera or essential thrombocythemia).

“Compounds of the disclosure” or “disclosed compounds” refer to any of the active compounds disclosed herein, including compounds targeting PIM kinase (i.e., Compound (1)), any of the compounds disclosed herein targeting JAK and/or ACVR or any compounds known in the art to have any of these activities, all of which are incorporated herein by reference.

“Drugs” or “disclosed drugs” refer to compounds targeting JAK and/or ACVR, for example ruxolitinib, fedratinib, pacritinib, momelotinib, luspatercept, or a pharmaceutically acceptable salt thereof, as well as ropeginterferon alfa-2b (an interferon) and navtemadelin (an MDM2 inhibitor) or their pharmaceutically acceptable salts, or combinations of two or more thereof.

“PIM kinase inhibitor,” as used herein, refers to a compound that inhibits the activity of a Proviral Insertion in Murine Lymphomas (PIM) kinase. PIM kinase refers to a family of serine/threonine kinases that regulate several signaling pathways that are fundamental to cancer development and progression. The PIM family includes PIM1, PIM2, and PIM3. A PIM inhibitor can have activity on all PIM family members or one or more subtypes of the PIM family. A PIM inhibitor can be selected for action on a specific subtype of the PIM family, for example a PIM inhibitor can act at a lower concentration on PIM1 of the PIM family than on other members of the PIM family. More specifically, a PIM inhibitor can selectively act on PIM1 compared to its action on, for example, PIM2. In some embodiments, a PIM inhibitor inhibits one or more PIM subtypes comprising PIM1.

A “PIM1 inhibitor” or a “PIM1 kinase inhibitor,” as used herein, refers to a compound that functions as an inhibitor to PIM1. In some embodiments, a PIM1 inhibitor is selective for PIM1 (e.g., acts at a lower concentration) compared to other PIM subtypes, particularly in comparison to PIM1 inhibition, in which case it can be referred to as a “selective PIM1 inhibitor” or “selective PIM1 kinase inhibitor.” A selective PIM1 inhibitor will commonly exhibit a PIM1/PIM2 IC₅₀ ratio of less than about 0.05, 0.045, 0.04, 0.035, 0.03, or 0.025, determined as described in Foulks, Neoplasia Vol. 16, No. 5, 2014. Alternatively, the selective PIM1 inhibitor will exhibit a PIM1/PIM2 IC₅₀ ratio of from about 0.001 to 0.05, from about 0.005 to 0.04, or from about 0.01 to 0.03.

A particular example of a selective PIM1 kinase inhibitor is Compound (1) having the following structure:

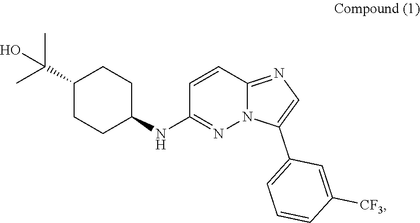 [SMILES: CC(C)(O)[C@H]1CC[C@H](Nc2ccc3ncc(-c4cccc(C(F)(F)F)c4)n3n2)CC1]

or a pharmaceutically acceptable salt thereof (a/k/a TP-3654 or cmpd 1). Another particular example of a selective PIM1 kinase inhibitor is Compound (2) having the following structure:

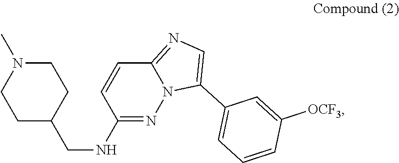 [SMILES: CN1CCC(CNc2ccc3ncc(-c4cccc(OC(F)(F)F)c4)n3n2)CC1]

or a pharmaceutically acceptable salt thereof (a/k/a SGI-1776 or cmpd 2). Additional examples are given by reference to publications in the body of this disclosure, which are herein incorporated by reference.

“JAK inhibitor,” as used herein, refers to a compound that inhibits the activity of a Janus kinase. Janus kinase refers to a family of intracellular, nonreceptor tyrosine kinases that transduce cytokine-mediated signals via the JAK-STAT pathway. The JAK family includes JAK1, JAK2, JAK3, and TYK2. A JAK inhibitor can have activity on all JAK family members or one or more subtypes of the JAK family. A JAK inhibitor can be selected for action on a specific subtype of the JAK family, for example, a JAK inhibitor can act at a lower concentration on JAK2 of the JAK family than on other members of the JAK family. More specifically, a JAK inhibitor can selectively act on JAK2 compared to its action on, for example, JAK1. In some embodiments, a JAK inhibitor inhibits one or more JAK subtypes comprising JAK2. In some embodiments, a JAK inhibitor inhibits one or more JAK subtypes comprising JAK1 and JAK2 (e.g., ruxolitinib).

In some embodiments, the JAK inhibitor may include ruxolitinib, tofacitinib, oclacitinib, baricitinib, filgotinib, gandotinib, lestaurtinib, momelotinib, pacritinib, PF-04965842, updacitinib, perficitinib, fedratinib, cucurbitacin I, CHZ868, decernotinib, CEP-33779, R348, fibotinib, ABT-494 which compounds are known in the art, or combinations of two or more thereof. In some embodiments, the JAK inhibitor may include BMS-911543, ASN002, itacitinib, NS-018, AZD1480, gandotinib, or combinations of two or more thereof. In some embodiments, the JAK inhibitor may include ruxolitinib, gandotinib, lestaurtinib, momelotinib, pacritinib, fedratinib, or combinations of two or more thereof. In some embodiments, the JAK inhibitor may include ruxolitinib, fedratinib, pacritinib, or momelotinib, or a pharmaceutically acceptable salt thereof, or combinations of two or more thereof. In some embodiments, the JAK inhibitor may include ruxolitinib or a pharmaceutically acceptable salt thereof (e.g., ruxolitinib phosphate). In some embodiments, the JAK inhibitor may include fedratinib or a pharmaceutically acceptable salt thereof. In some embodiments, the JAK inhibitor may include pacritinib or a pharmaceutically acceptable salt thereof. In some embodiments, the JAK inhibitor may include momelotinib or a pharmaceutically acceptable salt thereof.

A “JAK1 inhibitor” as used herein refers to a compound that functions as an inhibitor to JAK1. In some embodiments, a JAK1 inhibitor is selective for JAK1 (e.g., acts at a lower concentration) compared to other JAK subtypes.

A “JAK2 inhibitor” as used herein refers to a compound that functions as an inhibitor to JAK2. In some embodiments, a JAK2 inhibitor is selective for JAK2 (e.g., acts at a lower concentration) compared to other JAK subtypes.

“ACVR inhibitor,” as used herein refers to a compound that inhibits the activity of activin receptor. Activin receptor refers to a receptor which binds activin and belongs to the Transforming growth factor beta superfamily of ligands (TGFβ), which are involved in a host of physiological processes including, growth, cell differentiation, homeostasis, osteogenesis, apoptosis and many other functions. The two types of activin receptors are activin type 1 and activin type 2. Activin type 1 receptors transduce signals for a variety of TGFβ ligands and activin type 2 receptors modulate signals for TGFβ ligands. The three types of activin type I receptors are ACVR1, ACVR1B, and ACVR1C, which each bind to a specific type II receptor-ligand complex. The two types of activin type 2 receptors are ACVR2A and ACVR2B. An ACVR inhibitor can have activity on all activin receptor family members or one or more subtypes of the activin receptor family. An ACVR inhibitor can be selected for action on a specific subtype of the activin receptor family, for example, an ACVR inhibitor can act at a lower concentration on ACVR1 of the activin receptor family than on other members of the activin receptor family. In some embodiments, an ACVR inhibitor inhibits one or more activin receptors comprising ACVR1. In some embodiments, the ACVR inhibitor may include momelotinib, pacritinib, luspatercept, or pharmaceutically acceptable salts thereof, or combinations of two or more thereof. In some embodiments, the ACVR inhibitor may include momelotinib or a pharmaceutically acceptable salt thereof. In some embodiments, the ACVR inhibitor may include pacritinib or a pharmaceutically acceptable salt thereof. In some embodiments, the ACVR inhibitor may include luspatercept or a pharmaceutically acceptable salt thereof.

An “ACVR1 inhibitor” as used herein refers to a compound that functions as an inhibitor to ACVR1. In some embodiments, an ACVR1 inhibitor is selective for type 1 kinase activin A receptor (e.g., acts at a lower concentration) compared to other activin receptors.

In some embodiments, a compound may be an ACVR inhibitor and a JAK inhibitor.

Embodiments of the present disclosure also include administration of prodrugs of the disclosed compounds. “Prodrug” is meant to indicate a compound that may be converted under physiological conditions or by solvolysis to a biologically active compound of the disclosure. Thus, the term “prodrug” refers to a metabolic precursor of a compound of the disclosure that is pharmaceutically acceptable. A prodrug may be inactive when administered to a human subject in need thereof, but is converted in vivo to an active compound of the disclosure. Prodrugs are typically rapidly transformed in vivo to yield the parent compound of the disclosure, for example, by hydrolysis in blood. The prodrug compound often offers advantages of solubility, tissue compatibility or delayed release in a subject (see, Bundgard, H., Design of Prodrugs (1985), pp. 7 9, 21 24 (Elsevier, Amsterdam)). A discussion of prodrugs is provided in Higuchi, T., et al., A.C.S. Symposium Series, Vol. 14, and in Bioreversible Carriers in Drug Design, Ed. Edward B. Roche, American Pharmaceutical Association and Pergamon Press, 1987.

The term “prodrug” is also meant to include any covalently bonded carriers, which release the active compound of the disclosure in vivo when such prodrug is administered to a subject. Prodrugs of a compound of the disclosure may be prepared by modifying functional groups present in the compound of the disclosure in such a way that the modifications are cleaved, either in routine manipulation or in vivo, to the parent compound of the disclosure. Prodrugs include compounds of the disclosure wherein a hydroxy, amino or mercapto group is bonded to any group that, when the prodrug of the compound of the disclosure is administered to a subject, cleaves to form a free hydroxy, free amino or free mercapto group, respectively. Examples of prodrugs include, but are not limited to, acetate, formate and benzoate derivatives of alcohol or amide derivatives of amine functional groups in the compounds of the disclosure, and the like.

Embodiments of the disclosure are also meant to encompass administration of all pharmaceutically acceptable compounds of the disclosed compounds being isotopically-labeled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I. These radiolabeled compounds could be useful to help determine or measure the effectiveness of the compounds, by characterizing, for example, the site or mode of action, or binding affinity to a pharmacologically important site of action. Certain isotopically-labeled compounds of structure (I), (II) or (III), for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of structure (I), (II) or (III) can generally be prepared by conventional techniques known to those skilled in the art or by processes analogous to those described in the Preparations and Examples as set out below using an appropriate isotopically-labeled reagent in place of the non-labeled reagent.

Embodiments of the disclosure are also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, embodiments include compounds produced by a process comprising administering a compound of this disclosure to a human subject for a period of time sufficient to yield a metabolic product thereof. Such products are typically identified by administering a radio-labeled compound of the disclosure in a detectable dose to an animal, such as a rat, mouse, guinea pig, monkey, or to a human, allowing sufficient time for metabolism to occur, and isolating its conversion products from the urine, blood or other biological samples.

“Optional” or “optionally” means that the subsequently described event or circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable carrier, diluent or excipient” includes, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans.

“Pharmaceutically acceptable salt” includes both acid and base addition salts. Even if not specifically described in each instance, unless otherwise indicated (e.g., by the context), use of a therapeutic agent described herein (e.g., PIM kinase inhibitor, JAK kinase inhibitor) optionally comprises use of a pharmaceutically acceptable salt of the therapeutic agent instead of, or in addition to, the parent compound.

“Pharmaceutically acceptable acid addition salt” refers to those salts which retain the biological effectiveness and properties of the free bases, which are not biologically or otherwise undesirable, and which are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, and which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2 dimethylaminoethanol, 2 diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N ethylpiperidine, polyamine resins and the like. Particularly preferred organic bases are isopropylamine, diethylamine, ethanolamine, trimethylamine, dicyclohexylamine, choline and caffeine.

The compounds of the disclosure, or their pharmaceutically acceptable salts, may contain one or more asymmetric centers and may thus give rise to enantiomers, diastereomers, and other stereoisomeric forms that may be defined, in terms of absolute stereochemistry, as (R)- or (S)-, or as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centers giving rise to geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof, and includes “enantiomers,” which refers to two stereoisomers whose molecules are non-superimposable mirror images of one another.

A “pharmaceutical composition” refers to a formulation of a compound of the disclosure and a medium generally accepted in the art for the delivery of a biologically active compound to humans. Such a medium includes all pharmaceutically acceptable carriers, diluents or excipients for the compound of the disclosure.

“Effective amount” or “therapeutically effective amount” refers to that amount of a compound of the disclosure which, when administered to a human subject, is sufficient to effect treatment, as defined below, of a cancer, such as a MPN) in the subject. The amount of a compound of the disclosure which constitutes a “therapeutically effective amount” will vary depending on the compound, the condition and its severity, the manner of administration, and the age of the subject to be treated, but can be determined routinely by one of ordinary skill in the art having regard to his own knowledge and to this disclosure.

When an “effective amount” or “therapeutically effective amount” is referenced herein, it will be understood also to encompass a “safe and effective amount” or “therapeutically safe and effective amount.” To be effective, it will be understood that the intervention achieves a clinically meaningful response. In like manner, to be safe, it will be understood that the intervention is devoid of clinically meaningful adverse events, i.e. adverse events which would justify pausing or discontinuing therapy.

A “clinically significant” or “clinically meaningful” response means a response which is both statistically significant, and meaningful from a patient's, clinician's, or caregiver's perspective, typically based on a static measure such as CGI-S or a retrospective evaluation of improvement such as the CGI-C, as described generally in various publications of the United States Food and Drug Administration, including FDA 2018, FDA 2019, and FDA 2020. When a treatment or benefit is described herein, it will be understood that the treatment or benefit preferably shows clinically significant efficacy in a population of patients to a degree of statistical significance. A clinically “meaningful” response is synonymous with an “improvement.”

In one embodiment, a clinically meaningful response in a myeloproliferative neoplasm, or a clinically meaningful improvement in splenomegaly, means SVR25. In another embodiment, a clinically meaningful response in a myeloproliferative neoplasm, or a clinically meaningful improvement in splenomegaly, means SVR35. In another embodiment, a clinically meaningful response in a myeloproliferative neoplasm means TSS50. In another embodiment, a clinically meaningful response in a myeloproliferative neoplasm, or a clinically meaningful response in bone marrow fibrosis, means a fibrosis grade decrease of ≥1. In another embodiment, a clinically meaningful response in a myeloproliferative neoplasm means an increase in platelet counts by ≥50×10⁹/L. In another embodiment, a clinically meaningful response in a myeloproliferative neoplasm means an increase in platelet counts by ≥1 grade. In another embodiment, a clinically meaningful response in a myeloproliferative neoplasm means resolution of thrombocytopenia (commonly defined as platelet counts less than 150×10⁹/L). In another embodiment, a clinically meaningful response in a myeloproliferative neoplasm means an increase in Hgb of ≥2 g/dL. In another embodiment, a clinically meaningful response in a myeloproliferative neoplasm means an increase in Hgb by ≥1 grade. In another embodiment, a clinically meaningful response in a myeloproliferative neoplasm means resolution of anemia (commonly defined as Hgb<10 g/dL). In another embodiment, a clinically meaningful response in a myeloproliferative disorder means conversion of transfusion dependence to transfusion independence.

In other embodiments, the clinically meaningful improvement is based on probabilities of achieving TSS50 or SVR35, recognizing that not all patients will benefit equally. Thus, in various embodiments, when Compound (1) is administered as monotherapy, a clinically meaningful improvement in TSS50 will include probabilities of achieving TSS50≥30, 35, or 40 percent, and a clinically meaningful improvement in SVR35 will include probabilities of achieving SVR35≥20 or 25 percent. When Compound (1) is administered as combined therapy with momelotinib, a clinically meaningful improvement in TSS50 will include probabilities of achieving TSS50≥40 or 45 percent, and a clinically meaningful improvement in SVR35 will include probabilities of achieving SVR35≥20, 25 or 30 percent. When Compound (1) is administered as combined therapy with ruxolitinib, a clinically meaningful improvement in TSS50 will include probabilities of achieving TSS50≥50, 55, or 60 percent, and a clinically meaningful improvement in SVR35 will include probabilities of achieving SVR35≥40, 45, or 50 percent.

As used herein, “symptomatic burden” refers to constitutional or systemic (“whole body”) symptoms (e.g., fatigue, night sweats, weight loss, pruritus, fever, bone pain, joint pain, or a combination of two or more thereof).

Whenever a method of treatment is undertaken according to this disclosure, it will be understood that the method of treatment is undertaken for the purpose of achieving a clinically meaningful response.

In various embodiments, any of the foregoing clinically meaningful responses is observed for about 12 weeks or more, about 24 weeks or more, about 48 weeks or more, about 72 weeks or more, or about 96 weeks or more.

“Treating” or “treatment,” as used herein, covers the treatment of the disease or condition of interest in a human, having the disease or condition of interest, and includes: (i) preventing the disease or condition from occurring in the subject, in particular, when such subject is predisposed to the condition but has not yet been diagnosed as having it; (ii) inhibiting the disease or condition, i.e., arresting its development; (iii) relieving the disease or condition, i.e., causing regression of the disease or condition; or (iv) relieving the total symptoms resulting from the disease or condition, i.e., relieving pain without addressing the underlying disease or condition.

With respect to MF, “treating” or “treatment” includes achieving complete or partial remission, cytogenetic remission or molecular remission of the MF, and achieving clinical improvement, a hematological response (e.g., platelets, red blood cell, and/or white blood cells), a spleen response, a total symptoms score response or stable disease (collectively referred to as “improvement” of MF), (sometimes referred to as IWG-MRT response criteria) as those terms are defined in Tefferi, A., et al., Blood 2013, 122:1395-1398 (see, in particular, Table 1 of Tefferi), the relevant teachings of which are incorporated herein by reference in their entireties. In various embodiments, any of the foregoing improvements is observed for about 12 weeks or more, about 24 weeks or more, about 48 weeks or more, about 72 weeks or more, or about 96 weeks or more (i.e., the improvement is observed at the foregoing timepoints following initiation of treatment, but not necessarily at all or any timepoints before the foregoing timepoints). In some embodiments, any of the foregoing improvements is observed following treatment for about 12 weeks or more, about 24 weeks or more, about 48 weeks or more, about 72 weeks or more, or about 96 weeks or more (i.e., the improvement is not observed until the foregoing timepoints following initiation of treatment). In some embodiments, any of the foregoing improvements is observed following treatment for about 26 weeks or more, about 40 weeks or more, about 52 weeks or more, or about 80 weeks or more.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms has been identified by clinicians.

As used herein, the term “JAK-resistant” or “JAK-inhibitor resistant” in reference to a disease or condition refers to a human subject having the disease or condition and having been treated with a JAK inhibitor and exhibited an inadequate response or no response. In some embodiments, the MPN may be ruxolitinib-resistant.

As used herein, the term “inadequate” or “inadequate response” in reference to a drug treatment refers to a human subject having a relapsed and/or refractory response to the drug (e.g., ruxolitinib and/or fedratinib), a human subject who could not tolerate the drug (i.e., intolerant), a human subject that had a suboptimal response including an inferior spleen volume reduction, total symptom reduction, cytokine modulation, survival time, or a combination of two or more thereof. In some embodiments, inadequate response may refer to a lack of: bone marrow fibrosis reduction, and/or normalization or increased platelet count, and/or normalization or increased neutrophil count, and/or normalization or increased hemoglobin count, and/or transfusion improvement (including conversion into transfusion independence). In some embodiments, inadequate response may refer to side effects that require discontinuation of treatment with the JAK inhibitor.

“Poor bone marrow function” refers to various states in which the bone marrow is not functioning properly, including myelosuppressed states in which the bone marrow doesn't make enough blood cells or platelets, and is commonly manifested in conditions such as thrombocytopenia (low platelet count), anemia (low hemoglobin concentrations), or transfusion dependence. Thus, whenever a patient treated by the methods of the current disclosure is said to have poor bone marrow function, it will be understood that the patient can have thrombocytopenia (i.e. platelet counts are less than or equal to 150, 100, 75, or 50×10⁹/L, anemia (i.e. Hgb≤10, 9, or 8), or transfusion dependence, or a combination of such symptoms of poor bone marrow function.

“Transfusion independence” or “transfusion independent” refers to the patient needing no blood transfusion and an Hgb≥8 for at least twelve weeks. “Transfusion dependence” or “transfusion dependent” refers to a patient that is not transfusion independent. For example, a transfusion dependent patient may be in need of at least one blood transfusion in the previous 12 weeks. In some embodiments, a transfusion dependent patient may be in need of two or more blood transfusions in the previous 12 weeks. In other embodiments, the patient may be in need of one, two, or more blood transfusions in the previous 8 weeks. As used herein, “transfusion improvement” refers to decreased need for blood transfusion or blood part transfusion (e.g., red blood cells, white blood cells, and/or platelet transfusion). In some embodiments, the subject may become transfusion independent (i.e., no longer in need of blood or blood parts (e.g., red blood cells, white blood cells, and/or platelet transfusion), commonly based on the 12 weeks immediately preceding treatment.

In some embodiments, the subject may have relapsed following treatment with a drug (e.g., ruxolitinib and/or fedratinib). In some embodiments, “relapsed” or “relapsed response” to a drug refers to a human subject who has previously achieved≥35% decrease in spleen volume (a/k/a “SVR35”) and/or ≥50% decrease in the total symptoms score (a/k/a “TSS50”) during treatment with the drug (e.g., ruxolitinib), but then a loss of either of these responses during treatment. In some embodiments, the subject may have exhibited a refractory response to a drug (e.g., ruxolitinib and/or fedratinib). In some embodiments, “refractory” or “refractory response” to a drug refers to a human subject who never achieved≥35% decrease in spleen volume and/or ≥50% decrease in the total symptoms score during treatment with the drug.

In some embodiments, a subject has been “previously treated” with a JAK inhibitor. Previous treatment can last more than 3, 6, 9, or even 12 months before a decision is made to commence administration of Compound (1).

When a subject has been “previously treated” with a JAK inhibitor, and commences administration of Compound (1), administration of the JAK inhibitor can be continued or ceased, based on the medical judgment of the treating physician, and the nature of the subject's response to the JAK inhibitor. Thus, for example, if the subject has shown some response, and the response is suboptimal or plateaued, the physician might choose to continue administration of the JAK inhibitor in combination with Compound (1). When, however, the subject has suffered significant hematological toxicities from the JAK inhibitor, the subject is refractory to the JAK inhibitor, or the subject's disease has relapsed, the physician might choose to discontinue administration of the JAK inhibitor, and administer Compound (1) either as monotherapy or in combination with another JAK inhibitor and/or ACVR inhibitor or other MPN treatment.

Most commonly, when a subject has been “previously treated” with a JAK inhibitor, the JAK inhibitor will be ruxolitinib. When the JAK inhibitor is ceased, and Compound (1) is commenced in combination with another JAK inhibitor, Compound (1) will most commonly be co-administered with momelotinib.

In some embodiments, the subject may not have tolerated the drug (i.e., drug intolerance) (e.g., ruxolitinib and/or fedratinib). In some embodiments, drug intolerance refers to a subject's inability to tolerate adverse effects of the drug at therapeutic or subtherapeutic doses. Adverse effects include, but are not limited to, anemia, thrombocytopenia, transfusion dependence, neutropenia, bruising, dizziness, headache, urinary tract infection, tiredness, shortness of breath, weight gain, gas, diarrhea, constipation, herpes zoster, or combinations of two or more thereof.

In some embodiments, the subject may have had an inferior spleen volume reduction following treatment with a drug (e.g., ruxolitinib and/or fedratinib). In some embodiments, an inferior spleen volume reduction may refer to less than 35% spleen volume reduction (including less than about 30%, less than about 25%, less than about 20%, less than about 15%, less than about 10%, less than about 5%, less than about 1%, or no measurable reduction, based on the subject's baseline spleen volume (i.e., spleen volume prior to starting treatment). I.e., the subject does not achieve SVR35. In some embodiments, the spleen volume reduction may be measured 24 weeks after the first treatment dose.

In some embodiments, the subject may have had an inferior total symptom reduction following treatment with a drug (e.g., ruxolitinib and/or fedratinib). In some embodiments, an inferior response to a drug or an inferior total symptom reduction may refer to less than 50% total symptom reduction (including less than about 45%, less than about 40%, less than about 35%, less than about 30%, less than about 25%, less than about 20%, less than about 15%, less than about 10%, less than about 5%, less than about 1%, or no measurable reduction). I.e., the subject does not achieve TSS50. In some embodiments, the total symptom reduction may be measured 24 weeks (i.e., week 24) after the first treatment dose.

In some embodiments, the subject may have had an inferior cytokine modulation following treatment with a drug (e.g., ruxolitinib and/or fedratinib). In some embodiments, an inferior cytokine modulation may refer to a down-regulation<20%. In some embodiments, an inferior cytokine modulation may refer to a down-regulation≤15%. In some embodiments, an inferior cytokine modulation may refer to a down-regulation≤10%. In some embodiments, an inferior cytokine modulation may refer to the cytokine not returning to normal levels (i.e., cytokine level in a gender-matched, healthy cohort).

In some embodiments, the subject may have had an inferior survival time following treatment with a drug (e.g., ruxolitinib and/or fedratinib). In some embodiments, an inferior survival time may refer to a survival rate of less than 1 year following the first treatment dose.

As used herein, “normal levels” or “normalization” refers to the level of the measured component (e.g., platelet, white blood cell (e.g., neutrophil), red blood cells (e.g., hemoglobin), spleen volume, bone marrow fibrosis reduction, total symptom score, cytokine levels) in a gender-matched, healthy cohort. In some embodiments, normalization may result in transfusion improvement (including conversion into transfusion independence).

As used herein, the term “1-grade” or “1 grade” in reference to a subject's platelet count, hemoglobin count, neutrophil count, or bone marrow fibrosis refers to a change in the subject's grade from one grade to the subsequent grade. For example, the change from Grade 1 to Grade 2 is a change in 1-grade, whereas the change from Grade 1 to Grade 3 is a change in 2-grades. Accordingly, a 1-grade change may be a change from Grade 1 to Grade 2, from Grade 2 to Grade 3, or from Grade 3 to Grade 4.

As used herein, “Grade 1” in reference to a subject's platelet count refers to a platelet count of greater than or equal to 75×10⁹/L; “Grade 2” refers a subject with a platelet count less than 75×10⁹/L and greater than or equal to 50×10⁹/L; “Grade 3” refers to a subject with a platelet count less than 50×10⁹/L and greater than or equal to 25×10⁹/L; and Grade 4 is a subject with a platelet count less than 25×10⁹/L. Thus, higher platelet grades indicate further severe disease conditions. A term “up-grade” refers to change grade 1 to grade 2, 3, or 4, or from grade 2 to grade 3 or 4, or from grade 3 to grade 4. The term “up-grade” indicates worsening disease conditions from the platelet count aspect. The term “down-grade” refers to change grade from grade 4 to grade 3, 2, or 1, or from grade 3 to grade 2 or 1, or from grade 2 to 1. The term “down-grade” indicates improving disease conditions from the platelet count aspect.

As used herein, “Grade 1” in reference to a subject's neutrophil count refers to a neutrophil count of greater than or equal to 1500/L; “Grade 2” refers a subject with a neutrophil count less than 1500/L and greater than or equal to 1000/L; “Grade 3” refers to a subject with a neutrophil count less than 1000/L and greater than or equal to 500/L; and Grade 4 is a subject with a neutrophil count less than 500/L. Thus, higher neutrophil grades indicate further severe disease conditions. A term “up-grade” refers to change grade 1 to grade 2, 3, or 4, or from grade 2 to grade 3 or 4, or from grade 3 to grade 4. The term “up-grade” indicates worsening disease conditions from the neutrophil count aspect. The term “down-grade” refers to change grade from grade 4 to grade 3, 2, or 1, or from grade 3 to grade 2 or 1, or from grade 2 to 1. The term “down-grade” indicates improving disease conditions from the neutrophil count aspect.

As used herein, “Grade 1” in reference to a subject's hemoglobin count refers to a hemoglobin count of greater than or equal to 10.0 g/dL; “Grade 2” refers a subject with a hemoglobin count less than 10.0 g/dL and greater than or equal to 8.0 g/dL; “Grade 3” refers to a subject with a hemoglobin count less than 8.0 g/dL. Thus, higher hemoglobin grades indicate further severe disease conditions. A term “up-grade” refers to change grade 1 to grade 2 or 3, or from grade 2 to grade 3. The term “up-grade” indicates worsening disease conditions from the hemoglobin count aspect. The term “down-grade” refers to change grade from grade 3 to grade 2 or 1, or from grade 2 to grade 1. The term “down-grade” indicates improving disease conditions from the hemoglobin count aspect.

As used herein, “bone marrow fibrosis reduction” or “BMFR” or “reduction of bone marrow fibrosis” refers to an improvement or decrease in bone marrow fibrosis of 1 grade or more.

As used herein, “Grade 1” in reference to a subject's bone marrow fibrosis refers to a loose network of reticulin with many intersections, especially in perivascular areas. “Grade 2” refers to diffuse and dense increase in reticulin with extensive intersections, occasionally with focal bundles of thick fibers mostly consistent with collagen, and/or focal osteosclerosis. “Grade 3” refers to diffuse and dense increase in reticulin with extensive intersections and coarse bundles of thick fibers consistent with collagen, usually associated with osteosclerosis. Thus, higher bone marrow fibrosis grades indicate further severe disease conditions. A term “up-grade” refers to change grade 1 to grade 2, 3, or 4, or from grade 2 to grade 3 or 4, or from grade 3 to grade 4. The term “up-grade” indicates worsening disease conditions from the bone marrow fibrosis aspect. The term “down-grade” refers to change grade from grade 4 to grade 3, 2, or 1, or from grade 3 to grade 2 or 1, or from grade 2 to 1. The term “down-grade” indicates improving disease conditions from the bone marrow fibrosis aspect.

As used herein, “spleen volume” is evaluated using either CT or MRI imaging scan of the abdomen and determining the volume of the spleen during treatment. A subject with ≥20% decrease in spleen volume (i.e., spleen volume reduction) at the 24th week (i.e., week 24) compared to spleen volume prior to the first treatment dose is designated as having a spleen volume reduction in response to treatment of SVR20. In some embodiments, the subject may have a ≥35% decrease in spleen volume at the 24th week (i.e., week 24) compared to spleen volume prior to the first treatment dose is designated as having a spleen volume reduction in response to treatment of SVR35. In some embodiments, a subject may have a spleen volume decrease of at least about 35% (including at least about 40%, at least about 45%, at least about 50%, at least about 55%, at least about 60%, or at least about 65%) at the 24th week (i.e., week 24) compared to spleen volume prior to the first treatment dose.

In some embodiments, when the subject had an inadequate response to a JAK inhibitor, the patient may have a spleen volume decrease of less than 35%, less than 25%, less than 20%, or less than 15% at the 24th week (i.e., week 24) compared to spleen volume prior to the first treatment dose with the JAK inhibitor.

As used herein, “cytokine” refers to a small protein(s) important in cell signaling. Cytokines include chemokines, interferons, interleukins, lymphokines, and tumor necrosis factor.

As used herein, “cytokine modulation” refers to increased cytokine levels (i.e., up-regulation) or decreased cytokine levels (i.e., down-regulation) in the subject's serum compared to the subject's cytokine level prior to the first treatment dose. In some embodiments, the cytokine modulation in the subject may refer to the subject's cytokine returning to normal levels.

In some embodiments, the cytokine modulation in the subject may be an up-regulation. In some embodiments, the up-regulation may refer to an increase of a cytokine by ≥about 20%, ≥about 25%, ≥about 35%, ≥about 45%, ≥about 50%, ≥about 60%, ≥about 75%, ≥about 100%, ≥about 150%, ≥about 200%, ≥about 250%, ≥about 300%, or ≥about 500% compared to the cytokine level prior to the first treatment dose. In some embodiments, the up-regulation may refer to an increase of a cytokine by ≥about 25% compared to the cytokine level prior to the first treatment dose. In some embodiments, the up-regulation may refer to an increase of a cytokine by ≥about 50% compared to the cytokine level prior to the first treatment dose. In some embodiments, the cytokine up-regulation may be measured 2 hours, 4 hours, 6 hours, 8 hours, 10 hours, 12 hours, 14 hours, 16 hours, 18 hours, or 20 hours (i.e., day 0) after the first treatment dose. In some embodiments, the cytokine up-regulation may be measured 6 hours, 8 hours, 10 hours, or 12 hours (i.e., day 0) after the first treatment dose. In some embodiments, the cytokine up-regulation may be measured 24 hours (i.e., day 1) after the first treatment dose. In some embodiments, the cytokine up-regulation may be measured 4 weeks (i.e., week 4) after the first treatment dose. In some embodiments, the cytokine up-regulation may be measured 6 weeks (i.e., week 6) after the first treatment dose. In some embodiments, the cytokine up-regulation may be measured 8 weeks (i.e., week 8) after the first treatment dose. In some embodiments, the cytokine up-regulation may be measured 10 weeks (i.e., week 10) after the first treatment dose. In some embodiments, the cytokine up-regulation may be measured 12 weeks (i.e., week 12) after the first treatment dose.

In some embodiments, the cytokine modulation in the subject may be a down-regulation. In some embodiments, the down-regulation may refer to a decrease of a cytokine by ≥about 20%, ≥about 25%, ≥about 35%, ≥about 45%, ≥about 50%, ≥about 60%, ≥about 75%, ≥about 100%, ≥about 150%, ≥about 200%, ≥about 250%, ≥about 300%, or ≥about 500% compared to the cytokine level prior to the first treatment dose. In some embodiments, the down-regulation may refer to a decrease of a cytokine by ≥about 25% compared to the cytokine level prior to the first treatment dose. In some embodiments, the down-regulation may refer to a decrease of a cytokine by ≥about 30% compared to the cytokine level prior to the first treatment dose. In some embodiments, the down-regulation may refer to a decrease of a cytokine by ≥about 40% compared to the cytokine level prior to the first treatment dose. In some embodiments, the down-regulation may refer to a decrease of a cytokine by ≥about 50% compared to the cytokine level prior to the first treatment dose. In some embodiments, the down-regulation may refer to a decrease of a cytokine by ≥about 60% compared to the cytokine level prior to the first treatment dose. In some embodiments, the down-regulation may refer to a decrease of a cytokine by ≥about 70% compared to the cytokine level prior to the first treatment dose. In some embodiments, the down-regulation may refer to a decrease of a cytokine by ≥about 80% compared to the cytokine level prior to the first treatment dose. In some embodiments, the down-regulation may refer to a decrease of a cytokine such that the cytokine is at a normal level (i.e., cytokine level in a gender-matched, healthy cohort). In some embodiments, the cytokine down-regulation may be measured 2 hours, 4 hours, 6 hours, 8 hours, 10 hours, 12 hours, 14 hours, 16 hours, 18 hours, or 20 hours (i.e., day 0) after the first treatment dose. In some embodiments, the cytokine down-regulation may be measured 6 hours, 8 hours, 10 hours, or 12 hours (i.e., day 0) after the first treatment dose. In some embodiments, the cytokine down-regulation may be measured 24 hours (i.e., day 1) after the first treatment dose. In some embodiments, the cytokine down-regulation may be measured 4 weeks (i.e., week 4) after the first treatment dose. In some embodiments, the cytokine down-regulation may be measured 6 weeks (i.e., week 6) after the first treatment dose. In some embodiments, the cytokine down-regulation may be measured 8 weeks (i.e., week 8) after the first treatment dose. In some embodiments, the cytokine down-regulation may be measured 10 weeks (i.e., week 10) after the first treatment dose. In some embodiments, the cytokine down-regulation may be measured 12 weeks (i.e., week 12) after the first treatment dose. In some embodiments, the cytokine down-regulation may be measured 6 months after the first treatment dose. In some embodiments, the cytokine down-regulation may be measured 12 months after the first treatment dose. In some embodiments, the cytokine down-regulation may be measured 18 months after the first treatment dose. In some embodiments, the cytokine down-regulation may be measured 24 months after the first treatment dose.

In some embodiments, the cytokine modulation in the subject may be an up-regulation of certain cytokines and a down-regulation of other cytokines as defined above.

In some embodiments, the cytokine may include adiponectin, angiopoietin 1, angiotensin II, beta-2 microglobulin, bone morphogenetic protein (BMP) and subtypes thereof (e.g., BMP1, BMP6, and BMP7), bone morphogenetic protein Rcp2, brain-derived neurotrophic factor (BDNF), C-C motif chemokine ligand (CCL) and subtypes thereof (e.g., CCL14 and CCL21), cluster of differentiation 40 ligand (CD40L), cluster of differentiation 40 (CD40), colony stimulating factor (CSF1), complement factor D, C-reactive protein (CRP), C-X3-C motif chemokine receptor 1 (CX3CR1), C-X-C motif chemokine ligand (CXL) and subtypes thereof (e.g., CXL11, CXCL12, and CXCL16), Dickkopf WNT Signaling Pathway Inhibitor 1 (DKK1), endoglin, eotaxin and subtypes thereof (e.g., eotaxin-2 and eotaxin-3), epidermal growth factor (EGF), epidermal growth factor receptor (EGFR), erythropoietin (EPO), extracellular matrix protein tenascin-C (TN-C), extracellular newly identified receptor for advanced glycation end-products binding protein (EN-RAGE), ferritin, fibroblast growth-factor (FGF), Fms Related Receptor Tyrosine Kinase 3 (FLT3), Fms-related tyrosine kinase 3 ligand (FLT-3L), granulocyte-macrophage colony-stimulating factor (GM-CSF), growth colony stimulating factor (G-CSF), growth-regulated protein alpha (GRO-alpha), heparin-binding EGF-like growth factor (HB-EGF), hepatocyte growth factor (HGF), insulin like growth factor binding protein 2 (IGFBP2), intercellular adhesion molecule 1 (ICAM-1), interferon (IFN) and subtypes thereof (e.g., interferon gamma-inducible protein 9 (IP-9), interferon-alpha (IFN-alpha), interferon-gamma (IFNg), interferon-gamma inducible protein (IFN-g-IP), interferon-gamma inducible protein 10 (IP-10), interleukin (IL) and subfactors thereof (e.g., IL-1a, IL-1Ra IL-2, IL-2R, IL-2Ra, IL-4, IL-5, IL-5a, IL-6, IL-7, IL-8, IL-10, IL-11, IL-12, IL-12p40, IL-12p70, IL-13, IL-15, IL-17, IL-17A, IL-17F, IL-18, IL-22, IL-23, IL-1B, and IL-1RA), isocitrate dehydrogenase 1 (IDH1), isocitrate dehydrogenase 2 (IDH2), latency-associated peptide transforming growth factor-β1 (LAP TGF-b1), leptin (LEP), LIF interleukin 6 family cytokine (LIF), macrophage colony stimulating factor 1 (MCSF), macrophage derived chemokine (MDC), macrophage inflammatory protein and subtypes thereof (e.g., MIP-1, MIP-1b, MIP-1a, MIP-3a, and MIP-3b), macrophage-derived chemokine (MDC), matrix metalloproteinase and subtypes thereof (e.g., MMP-3 and MMP-9), monocyte chemotactic protein and subtypes thereof (e.g., MCP-1, MCP-2, MCP-3, MCP-4, and MCP-5), monokine-induced by gamma (MIG), myelin basic protein and subtypes thereof (e.g., MBP R2 and MBP7), myeloperoxidase (MPO), myoglobin, osteoprotegerin (OPG), pentraxin (PTX) and subtypes thereof (e.g., PTX3), plasma protein factor VII (factor VII), plasminogen activator inhibitor-1 (PAI-1), platelet factor 4 (PF-4), platelet-derived growth factor BB (PDGF-BB), pulmonary and activation-regulated chemokine (PARC), regulated on activation normal T cell expressed and secreted (RANTES), stem cell factor (SCF), ten-eleven translocation 2 (TET2), thrombopoietin (TPO), thymus and activation-regulated chemokine (TARC), tissue inhibitor of metalloproteinase-1 (TIMP-1), transforming growth factor-β (TGF-beta), transforming growth factor-β1 (TGF-b1), tumor necrosis factor and subtypes thereof (TNF-1, TNF-a, TNF-RI, and TNF-RII), urokinase plasminogen activator receptor (uPAR), vascular adhesion molecule (VCAM-1), vascular endothelial growth factor (VEGF) and subtypes thereof (VEGFb), vascular endothelial growth factor receptor (VGFR), α2-macroglobulin (A2M), or a combination of two or more thereof. In some embodiments, the cytokine may include EGFR, PDGF-BB, EPO, IP-10, TIMP-1, IL-2R-alpha, IL-1R-a, IL-12p40, IL-12p70, IL-15, MMP-3, MMP-9, VEGF, IL-2, IL-6, MIP-1 beta, TNF-a, TGF-beta, or a combination of two or more thereof. In some embodiments, the cytokine may include IL-1Ra, IL-6, IL-10, IL-12, IL-12, TGF-beta, EGFR, ferritin, GRO-a, MMP-9, PAI-1, RANTES, TIMP-1, TNFR-2, VCAM-1, or a combination of two or more thereof.

In some embodiments, the cytokine modulation is a down-regulation of adiponectin, angiopoietin 1, angiotensin II, beta-2 microglobulin, bone morphogenetic protein (BMP) and subtypes thereof (e.g., BMP1, BMP6, and BMP7), bone morphogenetic protein Rcp2, brain-derived neurotrophic factor (BDNF), C-C motif chemokine ligand (CCL) and subtypes thereof (e.g., CCL14 and CCL21), cluster of differentiation 40 ligand (CD40L), cluster of differentiation 40 (CD40), colony stimulating factor (CSF1), complement factor D, C-reactive protein (CRP), C-X3-C motif chemokine receptor 1 (CX3CR1), C-X-C motif chemokine ligand (CXL) and subtypes thereof (e.g., CXL11, CXCL12, and CXCL16), Dickkopf WNT Signaling Pathway Inhibitor 1 (DKK1), endoglin, eotaxin and subtypes thereof (e.g., eotaxin-2 and eotaxin-3), epidermal growth factor (EGF), epidermal growth factor receptor (EGFR), erythropoietin (EPO), extracellular matrix protein tenascin-C (TN-C), extracellular newly identified receptor for advanced glycation end-products binding protein (EN-RAGE), ferritin, fibroblast growth-factor (FGF), Fms Related Receptor Tyrosine Kinase 3 (FLT3), Fms-related tyrosine kinase 3 ligand (FLT-3L), granulocyte-macrophage colony-stimulating factor (GM-CSF), growth colony stimulating factor (G-CSF), growth-regulated protein alpha (GRO-alpha), heparin-binding EGF-like growth factor (HB-EGF), hepatocyte growth factor (HGF), insulin like growth factor binding protein 2 (IGFBP2), intercellular adhesion molecule 1 (ICAM-1), interferon (IFN) and subtypes thereof (e.g., interferon gamma-inducible protein 9 (IP-9), interferon-alpha (IFN-alpha), interferon-gamma (IFNg), interferon-gamma inducible protein (IFN-g-IP), interferon-gamma inducible protein 10 (IP-10), interleukin (IL) and subfactors thereof (e.g., IL-1a, IL-1Ra, IL-2, IL-2R, IL-2Ra, IL-4, IL-5, IL-5a IL-6, IL-7, IL-8, IL-10, IL-11, IL-12, IL-12p40, IL-12p70, IL-13, IL-15, IL-17, IL-17A, IL-17F, IL-18, IL-22, IL-23, IL-1B, and IL-1RA), isocitrate dehydrogenase 1 (IDH1), isocitrate dehydrogenase 2 (IDH2), latency-associated peptide transforming growth factor-β1 (LAP TGF-b1), leptin (LEP), LIF interleukin 6 family cytokine (LIF), macrophage colony stimulating factor 1 (MCSF), macrophage derived chemokine (MDC), macrophage inflammatory protein and subtypes thereof (e.g., MIP-1, MIP-1b, MIP-1a, MIP-3a, and MIP-3b), macrophage-derived chemokine (MDC), matrix metalloproteinase and subtypes thereof (e.g., MMP-3 and MMP-9), monocyte chemotactic protein and subtypes thereof (e.g., MCP-1, MCP-2, MCP-3, MCP-4, and MCP-5), monokine-induced by gamma (MIG), myelin basic protein and subtypes thereof (e.g., MBP R2 and MBP7), myeloperoxidase (MPO), myoglobin, osteoprotegerin (OPG), pentraxin (PTX) and subtypes thereof (e.g., PTX3), plasma protein factor VII (factor VII), plasminogen activator inhibitor-1 (PAI1), platelet factor 4 (PF-4), platelet-derived growth factor BB (PDGF-BB), pulmonary and activation-regulated chemokine (PARC), regulated on activation normal T cell expressed and secreted (RANTES), stem cell factor (SCF), ten-eleven translocation 2 (TET2), thrombopoietin (TPO), thymus and activation-regulated chemokine (TARC), tissue inhibitor of metalloproteinase-1 (TIMP1), transforming growth factor-β (TGF-beta), transforming growth factor-β1 (TGF-b1), tumor necrosis factor and subtypes thereof (TNF-1, TNF-a, TNF-RI, and TNF-RII), urokinase plasminogen activator receptor (uPAR), vascular adhesion molecule (VCAM1), vascular endothelial growth factor (VEGF) and subtypes thereof (VEGFb), vascular endothelial growth factor receptor (VGFR), α2-macroglobulin (A2M), or a combination of two or more thereof.

In some embodiments, the down-regulated cytokine may include B2MICG, BMP1, BMP6, BMP7, BMP-Rcp2, CD40L, CRP, ferritin, FGF, GCSF, GM-CSF, HGF, IFN, IL-1, IL-10, IL-12, IL-13, 1L-15, IL-17, IL-17A, IL-1B, IL-1RA, IL-2, IL-2R, IL-4, IL-5, IL-6, IL-7, IL-8, IFN, IFNa, IFNg, INF-g-IP, IP10, leptin, MBP R2, MBP7, MCP1, MIP1a, MIP1b, MCP2, MIG, MMP, PAL1, PDGF-BB, PTX, RANTES, TNF, TNF-1, TNF-a, TNF-RII, VCAM1, VEGF, VEGFb, VEGR, or a combination of two or more thereof. In some embodiments, the down-regulated cytokine may IL-6, IL-10, IL-12, IL-18, TGF-b, EGFR, Ferritin, GRO-a, IL-1RA, MMP-9, PAI-1, RANTES, TIMP-1, TNFR-2, VCAM-1, or a combination of two or more thereof. In some embodiments, the down-regulated cytokine may include MIG, EN-RAGE, MMP-9, CD40, FRTN, ICAM-1, TIMP-1, IL-18, SCF, MPO, IP-10, IL-2R-alpha, IL-8, MIP-1 beta, IL-12p40, EPO, or a combination of two or more thereof. In some embodiments, the down-regulated cytokine may include MCP-1, MIP-1 alpha, RANTES, IL-13, or a combination of two or more thereof. In some embodiments, the down-regulated cytokine may include IL-8, MMP-9, PAI-1, IL-2, CXL11, CSF1, UPAR, CRP, FLT-3L, EPO, INF-a, LEP, or a combination of two or more thereof. In some embodiments, the down-regulated cytokine may include IL-8, MMP-9, PAI-1, IL-2, or a combination of two or more thereof. In some embodiments, the down-regulated cytokine may include CXL11, CSF1, UPAR, or a combination of two or more thereof. In some embodiments, the down-regulated cytokine may include IL-1Ra, IL-6, IL-10, IL-12, IL-12, TGF-beta, EGFR, ferritin, GRO-a, MMP-9, PAI-1, RANTES, TIMP-1, TNFR-2, VCAM-1, or a combination of two or more thereof. In some embodiments, the down-regulated cytokine may include IL-6, IL-12p40, MMP9, EN-RAGE, or a combination of two or more thereof. In some embodiments, IL-6 may be down-regulated about ≥60%, IL-12p40 may be down-regulated about ≥75%, MMP9 may be down-regulated about ≥50%, EN-RAGE may be down-regulated about ≥60%, or a combination of two or more thereof.

As used herein, “up-titrating” or “up-titration” refers to increasing the amount of a compound or drug disclosed herein administered per day. This may include increasing the amount of the compound or drug at any given administration (e.g., increase a dose of 100 mg to 200 mg) and/or administering the compound or drug more times per day (e.g., increase the number of daily doses from once a day to twice a day). Up-titrating the dose amount and/or the number of daily doses of Compound (1) will result in an increase in the total daily dose of Compound (1) or the pharmaceutically acceptable salt thereof. Up-titrating the dose amount and/or the number of daily doses of ruxolitinib, fedratinib, pacritinib, momelotinib, luspatercept, ropeginterferon alfa-2b, or navtemadelin, will result in an increase in the total daily dose of the ruxolitinib, fedratinib, pacritinib, momelotinib, luspatercept, ropeginterferon alfa-2b, or navtemadelin, or the pharmaceutically acceptable salt thereof. In some embodiments, up-titration may begin from a given starting dose. In some embodiments, up-titration may occur every 4 weeks. In some embodiments, up-titration may continue until a maximum tolerated dose level is reaches or earlier if the subject exhibits MNP (e.g., MF) improvement such as bone marrow fibrosis reduction, total symptom reduction, cytokine modulation (e.g., cytokine reduction or normalization), normalization or increased platelet count, normalization or increased neutrophil count, normalization or increased hemoglobin count, transfusion improvement (including conversion into transfusion independence), spleen volume reduction, or a combination of two or more thereof. In some embodiments, bone marrow fibrosis, total symptom score, cytokine levels, platelet count, neutrophil count, hemoglobin count, and/or spleen volume may be measured every 2 to 4 weeks during treatment.

As used herein, “down-titrating” or “down-titration” refers to decreasing the amount of a compound or drug disclosed herein administered per day. This may include decreasing the amount of the compound or drug at any given administration and/or administering the compound or drug less times per day. For example, the amount of the compound or drug may be reduced by half (e.g., decrease a dose of 200 mg to 100 mg) and/or the number of daily administrations may be reduced by half (e.g., decrease the number of daily doses from twice a day to once a day). Down-titrating the dose amount and/or the number of daily doses of Compound (1) will result is a decrease in the total daily dose of Compound (1) or the pharmaceutically acceptable salt. Down-titrating the dose amount and/or the number of daily doses of ruxolitinib, fedratinib, pacritinib, momelotinib, luspatercept, ropeginterferon alfa-2b, or navtemadelin, will result is a decrease in the total daily dose of ruxolitinib, fedratinib, pacritinib, momelotinib, luspatercept, ropeginterferon alfa-2b, or navtemadelin, or the pharmaceutically acceptable salt thereof. At times, administration of Compound (1) may need to be interrupted. For example, stopping administration and restarting after a period of time. The interruption may last for a certain number of days, until an adverse effect has resolved, and/or until the treated subject's platelet count is at or above a certain amount. In some embodiments, a subject being treated with a stable dose of a JAK inhibitor and Compound (1) may have the administration of Compound (1) interrupted if new adverse event occurs. In some embodiments, administration of Compound (1) may be restarted after the adverse event has resolved. In some embodiments, if the adverse event reoccurs upon restarting administration of Compound (1), the total daily dose of Compound (1) may be reduced. In some embodiments, if the adverse event reoccurs upon restarting administration of Compound (1), administration of Compound (1) may be discontinued. In other embodiments, instead of an interruption, the total daily dose of Compound (1) may be reduced or remain the same depending on the severity of the adverse event. In some embodiments, if the adverse event is a platelet count reduction, resolution of the adverse event may be achieved when the subject's platelet count has increased to the platelet count when treated with only the JAK inhibitor (i.e., subject's baseline JAK inhibitor platelet count). In some embodiments, if the adverse event is a platelet count reduction, resolution of the adverse event may be achieved when the subject's platelet count has increased 1 grade (e.g., from grade 4 to grade 3). In some embodiments, if the adverse event is a platelet count reduction, resolution of the adverse event may be achieved when the subject's platelet count has increased at least 25×10⁹/L. In some embodiments, if the adverse event is a platelet count reduction, resolution of the adverse event may be achieved when the subject's platelet count has increased at least 35%.

The terms “not substantial,” “not substantially,” and “insubstantial” can be used herein to characterize the lack of adverse effects from the disclosed treatments. As used herein, “not substantial,” “not substantially,” or “unsubstantial” refers to about 45% or less. In some embodiments, not substantial refers to about 40% or less. In some embodiments, not substantial refers to about 35% or less. In some embodiments, not substantial refers to about 30% or less, about 25% or less, about 20% or less, about 15% or less, or about 10% or less. For example, in some embodiments, a decrease of a subject's platelet count by no more than 35% is not a substantial decrease (including ≤about 34%, ≤about 33%, ≤about 32%, ≤about 31%, ≤about 30%, ≤about 25%, ≤about 20%, ≤about 15%, K about 10%, or ≤about 5%). In some embodiments, an unsubstantial decrease of a subject's platelet count is by less than 1 grade (i.e., a platelet count decrease such that the subject remains in the same grade). In contrast, as used herein, “substantial” or “substantially” refers to about 25% or more. In some embodiments, substantial refers to about 30% or more, about 35% or more, about 40% or more, about 45% or more, about 50% or more, or about 55% or more.

As used herein, “stable” refers to little or no change such as a change of no more than about ±10%. In some embodiments, stable refers to about ±8%. In some embodiments, stable refers to about ±5%, about ±2%, or about ±1%. For example, in some embodiments, a subject's platelet count does not substantially decrease when it remains stable (i.e., ±10%). In some embodiments, a subject's platelet count, hemoglobin, neutrophil count, or bone marrow fibrosis is stable when the subject remains in the same grade (e.g., platelet count decreases, but subject remains in Grade-2). In some embodiments, when disease stability is achieved the monotherapy of the Compound (1) or the combination therapy that includes Compound (1) is continued at the dosage on which stability was achieved.

As used herein, “total symptom reduction” and similar phrases refers to a reduction of total symptom score of greater than or equal to about 50% (a/k/a TSS50) (including ≥about 55%, ≥about 60%, ≥about 65%, ≥about 70%, ≥about 75%, ≥about 80^{%}, ≥about 85%, ≥about 90%, ≥about 95%, ≥about 99%, or about 100%) from the subject's total symptom score immediately before treatment is begun. Symptoms used to determine total symptom score are those provided in the questionnaires MPN Symptoms Assessment Form (MPN-SAF) (Scherber R et al., Blood (2011) 118 (2): 401-408), the Myelofibrosis Symptom Assessment Form (MFSAF or MF-SAF) (Mesa R A et al, Res 2009, vol. 33(9) (pg. 1199-1203)), and the MPN-SAF TSS abbreviated version of the MPN-SAF (Emanuel R. et al., Journal of Clinical Oncology 30, no. 33 (Nov. 20, 2012) 4098-4103) (all incorporated herein by reference).

These questionnaires all ask the subject to score symptom on a number scale, and include questions related to symptoms such as fatigue, early satiety, abdominal discomfort, inactivity, problems with concentration, night sweats, pruritus, diffuse bone pain, pain under left ribs, fever, and unintentional weight loss. If needed to give precision to the foregoing questionnaires, the applicable version of the MPN-SAF and MPN-SAF TSS50 are those versions in effect on Oct. 30, 2022. Version 4 is the applicable version for MFSAF. As with any other test administered in this disclosure, a subject said to achieve a TSS50 on version 4 of the MFSAF need not have had version 4 administered, as long as the subject would have experienced TSS50 on version 4 if administered.

When an embodiment is based on a reduction in total symptoms in MF subjects on the MFSAF, it will be understood that a comparable reduction can be achieved on the MPN-SAF or MPN-SAF TSS in MPN subjects (particularly ET, PV, and MF subjects), and vice versa. In some embodiments, the total symptom reduction may be measured about 4 weeks after the first treatment dose and TSS50 observed at this time point. In some embodiments, the total symptom reduction may be measured about 12 weeks after the first treatment dose, and TSS50 observed at this time point. In some embodiments, the total symptom reduction may be measured about 24 weeks after the first treatment dose, and TSS50 observed at this time point. In some embodiments, the total symptom reduction may be measured about 36 weeks after the first treatment dose, and TSS50 observed at this time point.

As used herein, when two drugs are referred to as having “no overlapping” toxicities, it means that, based on each drug's toxicity profile when administered individually, a worker of ordinary skill in the art would not expect a clinically significant exacerbation of a particular toxicity when the two drugs are combined. One or multiple toxicities can be evaluated, but the toxicities of particular relevance to this disclosure are hematological toxicities such a platelet count, hemoglobin concentrations, and transfusion dependence. As thus defined, Compound (1) has no overlapping hematological toxicities with ruxolitinib or momelotinib in terms of thrombocytopenia, neutropenia, or anemia when the drugs are combined at the dose levels reported in Table V.

As used herein, “increased neutrophil count” or “increased neutrophil” refers to an increase in neutrophils or an increase in white blood cells.

As used herein, “increased hemoglobin count” or “increased hemoglobin” refers to an increase in hemoglobin or an increase in red blood cells.

As used herein, “about” refers to +10% of the given value. In some embodiments, about may be ±5% of the given value. In some embodiments, about may be ±2% of the given value. All numeric figures reported herein can be qualified by the term “about,” and further expressed based on the foregoing plus or minus percentages.

### I. Medical Treatments

In one aspect the disclosure provides a method for treating a myeloproliferative neoplasm in a human subject in need thereof, comprising administering to the subject a therapeutically effective amount of Compound (1) or a pharmaceutically acceptable salt thereof, wherein the subject has poor bone marrow functioning characterized by: thrombocytopenia (e.g. a platelet count of ≤150, 100, 75, 50, or 25×10⁹/); anemia (e.g. a Hgb level≤10, 9, or 8 g/dL); transfusion dependence; or a combination thereof, and wherein the treatment induces a clinically meaningful response in the myeloproliferative neoplasm.

In another aspect the disclosure provides a method for treating a myeloproliferative neoplasm in a human subject in need thereof, comprising administering to the subject a therapeutically effective amount of a selective PIM1 inhibitor, wherein the subject has poor bone marrow functioning characterized by: thrombocytopenia (e.g. a platelet count of ≤150, 100, 75, 50, or 25×10⁹/L); anemia (e.g. a Hgb level≤10, 9, or 8 g/dL); transfusion dependence; or a combination thereof, wherein the treatment induces a clinically meaningful response in the myeloproliferative neoplasm.

In another aspect the disclosure provides a method of treating a myeloproliferative neoplasm in a human subject in need thereof, wherein the subject has previously been treated by a JAK inhibitor for the myeloproliferative neoplasm and optionally has relapsed disease or is refractory to the JAK inhibitor, comprising administering to the subject a therapeutically effective amount of Compound (1) or a pharmaceutically acceptable salt thereof, wherein the treatment induces a clinically meaningful response in the myeloproliferative neoplasm.

In another aspect the disclosure provides a method of treating a myeloproliferative neoplasm by inducing TSS50 in a human subject in need thereof, comprising administering to the subject a therapeutically effective amount of Compound (1) or a pharmaceutically acceptable salt thereof, wherein the myeloproliferative neoplasm is treated by inducing TSS50 in the subject, optionally at a time point between and including 24 and 48 weeks.

In another aspect the disclosure provides a method of treating a myeloproliferative neoplasm in a human subject in need thereof, comprising administering to the subject Compound (1) or a pharmaceutically acceptable salt thereof, and ruxolitinib or momelotinib or a pharmaceutically acceptable salt thereof in amounts that induce no overlapping hematological toxicities (e.g., anemia and/or thrombocytopenia and/or transfusion dependence), wherein the treatment induces a clinically meaningful response in the myeloproliferative neoplasm.

In another aspect the disclosure provides a method of treating a myeloproliferative neoplasm in a human subject in need thereof, comprising administering to the subject a selective PIM1 inhibitor and ruxolitinib or momelotinib or a pharmaceutically acceptable salt thereof in amounts that induce no overlapping hematological toxicities (e.g., anemia and/or thrombocytopenia and/or transfusion dependence), wherein the treatment induces a clinically meaningful response in the myeloproliferative neoplasm.

In another aspect the disclosure provides a method of inducing a probability of SVR35 in a human subject affected by a myeloproliferative neoplasm≥15% or 20%, wherein the subject has previously been treated by a JAK inhibitor for the myeloproliferative neoplasm and optionally has relapsed disease or is refractory to the JAK inhibitor, comprising administering to the subject a therapeutically effective amount of Compound (1) or a pharmaceutically acceptable salt thereof, wherein the treatment induces a probability of SVR35 in the human subject≥15% or 20%. The foregoing probabilities are absolute probabilities, without reference to any comparator. The foregoing probabilities further assume the subject remains on treatment.

In another aspect the disclosure provides a method of inducing a probability of TSS50 in a human subject affected by a myeloproliferative neoplasm≥30%, 35%, 40%, 45%, or 50%, wherein the subject has previously been treated by a JAK inhibitor for the myeloproliferative neoplasm and optionally has relapsed disease or is refractory to the JAK inhibitor, comprising administering to the subject a therapeutically effective amount of Compound (1) or a pharmaceutically acceptable salt thereof, wherein the treatment induces a probability of TSS50 in the human subject≥30%, 35%, 40%, 45%, or 50%. The foregoing probabilities are absolute probabilities, without reference to any comparator. The foregoing probabilities further assume the subject remains on treatment.

In one aspect, the present technology provides a method for treating a myeloproliferative neoplasm in a human subject in need thereof, wherein the method includes administering to the subject a therapeutically effective amount of Compound (1) or a pharmaceutically acceptable salt thereof. In some embodiments, the subject has a platelet count of less than about 200×10⁹/L (e.g., less than about 150, 100, 75, 50, or 25×10⁹/L; less than 150, 100, 75, 50, or 25×10⁹/L; less than 50 or 25×10⁹/L; or less than 25×10⁹L).

In another aspect, the present technology provides a method for treating a myeloproliferative neoplasm in a human subject in need thereof, comprising administering to the subject a therapeutically effective amount of Compound (1) or a pharmaceutically acceptable salt thereof, wherein the subject has had one or more previous treatments comprising Compound (1) or the pharmaceutically acceptable salt. In response to the treating, the subject exhibits (a) a platelet count reduction of less than 1 grade compared to a baseline platelet grade, (b) a platelet count reduction of less than or equal to 25×10⁹/L compared to a baseline platelet count, and/or (c) a platelet count reduction of less than or equal to 35% compared to a baseline platelet count. The platelet count reduction is determined by a change comparing the baseline platelet count and a second platelet count. The baseline platelet count is the platelet count determined within one week prior to administering Compound (1) or the pharmaceutically acceptable salt. The second platelet count is determined at least 2 weeks after administering Compound (1) or the pharmaceutically acceptable salt.

In another aspect, the present technology provides a method for treating a myeloproliferative neoplasm in a human subject in need thereof, wherein the method includes a combination therapy comprising administering to the subject a therapeutically effective amount of Compound (1) or a pharmaceutically acceptable salt thereof; and a JAK inhibitor or a pharmaceutically acceptable salt thereof and/or an ACVR inhibitor or a pharmaceutically acceptable salt thereof. In some embodiments, the JAK inhibitor and ACVR inhibitor may be the same compound. In some embodiments, the subject has a platelet count of greater than or equal to about 10×10⁹/L. In some embodiments, the subject has a platelet count of greater than or equal to about 15×10⁹/L. In some embodiments, the subject has a platelet count of greater than or equal to about 20×10⁹/L. In some embodiments, the subject has a platelet count of greater than or equal to about 25×10⁹/L. In some embodiments, the subject has a platelet count of less than about 200×10⁹/L. In some embodiments, the subject has a platelet count of less than about 150×10⁹/L. In some embodiments, the subject has a platelet count of less than about 100×10⁹/L. In some embodiments, the subject has a platelet count of less than about 50×10⁹/L. In some embodiments, the subject has a platelet count of less than 25×10⁹/L. In some embodiments, the subject has a platelet count of greater than or equal to about 10×10⁹/L and less than or equal to about 200×10⁹/L.

In another aspect, the present technology provides a method for treating a myeloproliferative neoplasm in a human subject in need thereof, wherein the method includes a combination therapy comprising administering to the subject a therapeutically effective amount of Compound (1) or a pharmaceutically acceptable salt thereof; and ruxolitinib, fedratinib, pacritinib, momelotinib, luspatercept, ropeginterferon alfa-2b, navtemadelin, or a pharmaceutically acceptable salt thereof. In some embodiments, the subject has a platelet count of greater than or equal to about 10×10⁹/L. In some embodiments, the subject has a platelet count of greater than or equal to about 15×10⁹/L. In some embodiments, the subject has a platelet count of greater than or equal to about 20×10⁹/L. In some embodiments, the subject has a platelet count of greater than or equal to about 25×10⁹/L. In some embodiments, the subject has a platelet count of less than about 200×10⁹/L. In some embodiments, the subject has a platelet count of less than about 150×10⁹/L. In some embodiments, the subject has a platelet count of less than about 100×10⁹/L. In some embodiments, the subject has a platelet count of less than about 50×10⁹/L. In some embodiments, the subject has a platelet count of less than 25×10⁹/L. In some embodiments, the subject has a platelet count of greater than or equal to about 10×10⁹/L and less than or equal to about 200×10⁹/L.

In another aspect, the present technology provides a method for treating myeloproliferative neoplasm in a human subject in need thereof, comprising administering to the subject a therapeutically effective amount of Compound (1) or a pharmaceutically acceptable salt thereof, and ruxolitinib, fedratinib, pacritinib, momelotinib, luspatercept, ropeginterferon alfa-2b, navtemadelin, or a pharmaceutically acceptable salt thereof, wherein the subject has a baseline platelet count less than or equal to 200×10⁹/L and/or greater than or equal to 10×10⁹/L. The ruxolitinib or the pharmaceutically acceptable salt is in a dose of about 5 mg to about 25 mg; fedratinib or the pharmaceutically acceptable salt is in a dose of about 200 mg to about 500 mg; pacritinib or the pharmaceutically acceptable salt is in a dose of about 100 mg to about 200 mg; momelotinib or the pharmaceutically acceptable salt is in a dose of about 50 mg to about 200 mg; luspatercept or the pharmaceutically acceptable dose is in a dose of about 0.5 mg/kg to 1.75 mg/kg based on the subject's body weight; ropeginterferon alfa-2b or the pharmaceutically acceptable salt is in a dose of about 50 to 500 μg (e.g., about 50 to 150 μg, about 100 to 300 μg, about 200 to 400 μg, or about 300 to 500 μg; and navtemadelin or the pharmaceutically acceptable salt is in a dose of about 100 to 300 mg (e.g., about 100 to 150 mg, about 150 to 300 mg, or 120 to 240 mg).

In some embodiments, the subject has a platelet count of greater than or equal to about 10×10⁹/L. In some embodiments, the subject has a platelet count of greater than or equal to about 15×10⁹/L. In some embodiments, the subject has a platelet count of greater than or equal to about 20×10⁹/L. In some embodiments, the subject has a platelet count of greater than or equal to about 25×10⁹/L. In some embodiments, the subject has a platelet count of less than about 200×10⁹/L. In some embodiments, the subject has a platelet count of less than about 150×10⁹/L. In some embodiments, the subject has a platelet count of less than about 100×10⁹/L. In some embodiments, the subject has a platelet count of less than about 50×10⁹/L. In some embodiments, the subject has a platelet count of less than 25×10⁹/L. In some embodiments, the subject has a platelet count of greater than or equal to about 10×10⁹/L and less than or equal to about 200×10⁹/L.

In another aspect, the present technology provides a method of treating a myeloproliferative neoplasm in a human subject in need thereof, without causing a clinically significant risk of adverse events, comprising: administering to the subject ruxolitinib fedratinib, pacritinib, momelotinib, luspatercept, ropeginterferon alfa-2b, navtemadelin, or a pharmaceutically acceptable salt thereof for a period of up to about 36 months; determining or having determined if the subject had an adverse event during the treatment period; reducing the dose amount from about 33% to about 80%, or temporarily stopping the dose; and administering to the subject a therapeutically effective amount of Compound (1) or a pharmaceutically acceptable salt thereof. In some embodiments, the subject has a platelet count of greater than or equal to about 10×10⁹/L. In some embodiments, the subject has a platelet count of greater than or equal to about 15×10⁹/L. In some embodiments, the subject has a platelet count of greater than or equal to about 20×10⁹/L. In some embodiments, the subject has a platelet count of greater than or equal to about 25×10⁹/L. In some embodiments, the subject has a platelet count of less than about 200×10⁹/L. In some embodiments, the subject has a platelet count of less than about 150×10⁹/L. In some embodiments, the subject has a platelet count of less than about 100×10⁹/L. In some embodiments, the subject has a platelet count of less than about 50×10⁹/L. In some embodiments, the subject has a platelet count of less than 25×10⁹/L. In some embodiments, the subject has a platelet count of greater than or equal to about 10×10⁹/L and less than or equal to about 200×10⁹/L.

In another aspect, the present technology provides a method of treating a myeloproliferative neoplasm in a human subject in need thereof while the subject is undergoing a ruxolitinib, fedratinib, pacritinib, momelotinib, luspatercept, ropeginterferon alfa-2b, or navtemadelin, therapy, comprising: administering to the subject a therapeutically effective amount of Compound (1) or a pharmaceutically acceptable salt thereof; and continuing to administer to the subject the therapeutically effective amount of Compound (1) or the pharmaceutically acceptable salt, during which the subject either (a) does not receive the therapy (e.g., JAK therapy such as ruxolitinib), or (b) receives a reduced amount of the therapy (e.g., JAK therapy such as ruxolitinib). In some embodiments, the subject has a platelet count of greater than or equal to about 10×10⁹/L. In some embodiments, the subject has a platelet count of greater than or equal to about 15×10⁹/L. In some embodiments, the subject has a platelet count of greater than or equal to about 20×10⁹/L. In some embodiments, the subject has a platelet count of greater than or equal to about 25×10⁹/L. In some embodiments, the subject has a platelet count of less than about 200×10⁹/L. In some embodiments, the subject has a platelet count of less than about 150×10⁹/L. In some embodiments, the subject has a platelet count of less than about 100×10⁹/L. In some embodiments, the subject has a platelet count of less than about 50×10⁹/L. In some embodiments, the subject has a platelet count of less than 25×10⁹/L. In some embodiments, the subject has a platelet count of greater than or equal to about 10×10⁹/L and less than or equal to about 200×10⁹/L.

In another aspect, the present technology provides a method for treating a myeloproliferative neoplasm in a human subject in need thereof, the method includes administering to the subject Compound (1) or a pharmaceutically acceptable salt thereof; and an ACVR inhibitor (e.g., ACVR1 inhibitor), wherein the subject has a platelet count of less than 200×10⁹/L and/or greater than or equal to 10×10⁹/L. The ACVR1 inhibitor may be in a dose of about 50 mg to about 250 mg. In some embodiments, the subject has a platelet count of greater than or equal to about 10×10⁹/L. In some embodiments, the subject has a platelet count of greater than or equal to about 15×10⁹/L. In some embodiments, the subject has a platelet count of greater than or equal to about 20×10⁹/L. In some embodiments, the subject has a platelet count of greater than or equal to about 25×10⁹/L. In some embodiments, the subject has a platelet count of less than about 200×10⁹/L. In some embodiments, the subject has a platelet count of less than about 150×10⁹/L. In some embodiments, the subject has a platelet count of less than about 100×10⁹/L. In some embodiments, the subject has a platelet count of less than about 50×10⁹/L. In some embodiments, the subject has a platelet count of less than 25×10⁹/L. In some embodiments, the subject has a platelet count of greater than or equal to about 10×10⁹/L and less than or equal to about 200×10⁹/L.

In another aspect, the present technology provides a method for treating myeloproliferative neoplasm in a human subject in need thereof, comprising administering to the subject a therapeutically effective amount of Compound (1) or a pharmaceutically acceptable salt thereof, and a JAK inhibitor, wherein the subject has a platelet count of greater than or equal to about 10×10⁹/L and/or a platelet count of less than about 200×10⁹/L. The JAK inhibitor may be in a dose of about 5 mg to about 25 mg, about 200 mg to about 500 mg, or about 100 mg to about 200 mg. In some embodiments, the subject has a platelet count of greater than or equal to about 10×10⁹/L. In some embodiments, the subject has a platelet count of greater than or equal to about 15×10⁹/L. In some embodiments, the subject has a platelet count of greater than or equal to about 20×10⁹/L. In some embodiments, the subject has a platelet count of greater than or equal to about 25×10⁹/L. In some embodiments, the subject has a platelet count of less than about 200×10⁹/L. In some embodiments, the subject has a platelet count of less than about 150×10⁹/L. In some embodiments, the subject has a platelet count of less than about 100×10⁹/L. In some embodiments, the subject has a platelet count of less than about 50×10⁹/L. In some embodiments, the subject has a platelet count of less than 25×10⁹/L. In some embodiments, the subject has a platelet count of greater than or equal to about 10×10⁹/L and less than or equal to about 200×10⁹/L.

In another aspect, the present technology provides a pharmaceutical composition comprising a pharmaceutically acceptable carrier or excipient; Compound (1) or a pharmaceutically acceptable salt thereof; and ruxolitinib, fedratinib, pacritinib, momelotinib, luspatercept, ropeginterferon alfa-2b, or navtemadelin, or a pharmaceutically acceptable salt thereof.

In another aspect, the present technology provides a kit comprising Compound (1) or a pharmaceutically acceptable salt thereof; ruxolitinib, fedratinib, pacritinib, momelotinib, luspatercept, ropeginterferon alfa-2b, or navtemadelin, or a pharmaceutically acceptable salt thereof; and written instructions for administering Compound (1) or a pharmaceutically acceptable salt thereof, in combination with the ruxolitinib, fedratinib, pacritinib, momelotinib, luspatercept, ropeginterferon alfa-2b, or navtemadelin, or a pharmaceutically acceptable salt thereof, to treat a myeloproliferative neoplasm (e.g., MF).

In some embodiments, any of the methods disclosed herein may result in the subject exhibiting bone marrow fibrosis reduction and/or total symptom score reduction and/or cytokine modulation (e.g., cytokine reduction or normalization), and/or normalization or increased platelet count, and/or normalization or increased neutrophil count, and/or normalization or increased hemoglobin count, and/or transfusion improvement (including conversion into transfusion independence) and/or spleen volume reduction. In some embodiments, any of the methods disclosed herein may result in the subject exhibiting increased survival (≥1 year) and/or decreased rate of leukemia transformation and/or transplant eligibility.

In some embodiments, any of the methods disclosed herein may result in the subject exhibiting (a) a platelet count reduction of less than 1 grade compared to a baseline platelet grade, (b) a platelet count reduction of less than or equal to 25×10⁹/L compared to a baseline platelet count, and/or (c) a platelet count reduction of less than or equal to 35% compared to a baseline platelet count. The platelet count reduction is determined by a change comparing the baseline platelet count and a second platelet count. The baseline platelet count is the platelet count determined within one week prior to administering Compound (1) or the pharmaceutically acceptable salt. The second platelet count is determined at least 2 weeks after administering Compound (1) or the pharmaceutically acceptable salt.

In some embodiments, any of the methods disclosed herein may result in the subject exhibiting (a) a neutrophil count reduction of less than 1 grade compared to a baseline neutrophil grade, (b) a neutrophil count reduction of less than or equal to 500/L compared to a baseline neutrophil count, and/or (c) a neutrophil count reduction of less than or equal to 35% compared to a baseline neutrophil count. The neutrophil count reduction is determined by a change comparing the baseline neutrophil count and a second neutrophil count. The baseline neutrophil count is the neutrophil count determined within one week prior to administering Compound (1) or the pharmaceutically acceptable salt. The second neutrophil count is determined at least 2 weeks after administering Compound (1) or the pharmaceutically acceptable salt.

In some embodiments, any of the methods disclosed herein may result in the subject exhibiting (a) a hemoglobin count reduction of less than 1 grade compared to a baseline hemoglobin grade, (b) a hemoglobin count reduction of less than or equal to 5.0 g/dL compared to a baseline hemoglobin count, and/or (c) a hemoglobin count reduction of less than or equal to 35% compared to a baseline hemoglobin count. The hemoglobin count reduction is determined by a change comparing the baseline hemoglobin count and a second hemoglobin count. The baseline hemoglobin count is the hemoglobin count determined within one week prior to administering Compound (1) or the pharmaceutically acceptable salt. The second hemoglobin count is determined at least 2 weeks after administering Compound (1) or the pharmaceutically acceptable salt.

Accordingly, in one embodiment a method for treating myeloproliferative neoplasms in a human subject in need thereof is provided, the method comprising administering to the subject an effective amount of a PIM kinase inhibitor. Another embodiment provides a method for decreasing proliferation of hematopoietic cells in a subject, the method comprising contacting the cells with a PIM kinase inhibitor (e.g., an effective amount of a PIM kinase inhibitor).

Another embodiment provides a method for treating a subject having or at risk of developing an MPN such as MF, the method comprising administering to the subject a composition comprising a therapeutically effective amount of Compound 1, or a pharmaceutically acceptable salt thereof.

In some embodiments, the methods described herein involve identifying a subject being at risk of developing an MPN such as MF. In some embodiments, the methods described herein further include administering Compound 1, or a pharmaceutically acceptable salt thereof (e.g., an effective amount of Compound 1, or a pharmaceutically acceptable salt thereof), to a subject identified as being at risk of developing an MPN such as MF. In some embodiments, the methods further include administering Compound 1, or a pharmaceutically acceptable salt thereof (e.g., an effective amount of Compound 1, or a pharmaceutically acceptable salt thereof), to a subject suspected to have an MPN such as MF.

In some embodiments, provided are methods for prophylactically treating a an MPN such as MF comprising administering Compound 1, or a pharmaceutically acceptable salt thereof (e.g., an effective amount of Compound 1, or a pharmaceutically acceptable salt thereof), to a human subject in need thereof. In some embodiments, provided are methods for treating and MPN such as MF comprising administering Compound 1, or a pharmaceutically acceptable salt thereof (e.g., an effective amount of Compound 1, or a pharmaceutically acceptable salt thereof), to a human subject in need thereof.

In some embodiments, provided are methods for preventing and MPN such as MF comprising administering Compound 1, or a pharmaceutically acceptable salt thereof (e.g., an effective amount of Compound 1, or a pharmaceutically acceptable salt thereof), to a human subject in need thereof.

In some embodiments, provided are methods for inhibiting formation or deposition of MF tissue, the method including contacting MF tissue with Compound 1, or a pharmaceutically acceptable salt thereof, in an amount sufficient to inhibit formation or deposition of MF tissue.

In some embodiments, provided herein is a method for treating a subject having or at risk of developing an MPN such as MF, the method comprising administering to the subject in need thereof a therapeutically effective amount of Compound 1, or a pharmaceutically acceptable salt thereof.

In some embodiments, provided herein is a method for treating a myeloproliferative neoplasm in a human subject in need thereof, the method comprising administering an effective amount of a PIM kinase inhibitor. In some embodiments, the PIM kinase inhibitor is a PIM1 kinase inhibitor.

In some embodiments, provided herein is a method for treating a myeloproliferative neoplasm in a human subject in need thereof, comprising administering to the subject, a therapeutically effective amount of Compound (1) or a pharmaceutically acceptable salt thereof, wherein the subject has a baseline platelet count of less than or equal to 200×10⁹/L.

In some embodiments, the baseline platelet count is less than or equal to about 150×10⁹/L. In some embodiments, the baseline platelet count is less than or equal to about 100×10⁹/L. In some embodiments, the baseline platelet count is less than or equal to about 50×10⁹/L. In some embodiments, the baseline platelet count is less than or equal to about 25×10⁹/L. In some embodiments, the baseline platelet count is greater than or equal to about 5, 10, 15, 20, 25, or 35×10⁹/L.

In some embodiments, the subject has a platelet count of greater than or equal to about 10×10⁹/L. In some embodiments, the subject has a platelet count of greater than or equal to about 15×10⁹/L. In some embodiments, the subject has a platelet count of greater than or equal to about 20×10⁹/L. In some embodiments, the subject has a platelet count of greater than or equal to about 25×10⁹/L. In some embodiments, the subject has a platelet count of less than about 200×10⁹/L. In some embodiments, the subject has a platelet count of less than about 150×10⁹/L. In some embodiments, the subject has a platelet count of less than about 100×10⁹/L. In some embodiments, the subject has a platelet count of less than about 50×10⁹/L. In some embodiments, the subject has a platelet count of less than 25×10⁹/L. In some embodiments, the subject has a platelet count of greater than or equal to about 10×10⁹/L and less than or equal to about 200×10⁹/L.

In some embodiments, the method comprises continuing administering to the subject a therapeutically effective amount of Compound (1) or the pharmaceutically acceptable salt, wherein the subject has a platelet count reduction of (a) less than 1 grade, or (b) less than or equal to 25×10⁹/L, and/or (c) less than or equal to 35%, and the platelet count reduction is determined by a change in the baseline platelet count determined within one week prior to administering Compound (1) or the pharmaceutically acceptable salt and a second platelet count determined at least 2 weeks after administering. In some embodiments, the subject has a platelet count reduction of (a) less than 1 grade. In some embodiments, the subject has a platelet count reduction of (b) less than or equal to 25×10⁹/L. In some embodiments, the subject has a platelet count reduction of (c) less than or equal to 35%.

In some embodiments, the method comprises continuing administering to the subject a therapeutically effective amount of Compound (1) or the pharmaceutically acceptable salt, wherein the subject has a neutrophil count reduction of (a) less than 1 grade compared to a baseline neutrophil grade, (b) less than or equal to 500/L compared to a baseline neutrophil count, and/or (c) less than or equal to 35% compared to a baseline neutrophil count, and the neutrophil count reduction is determined by a change in the baseline neutrophil count determined within one week prior to administering Compound (1) or the pharmaceutically acceptable salt and a second neutrophil count determined at least 2 weeks after administering. In some embodiments, the subject has a neutrophil count reduction of (a) less than 1 grade. In some embodiments, the subject has a neutrophil count reduction of (b) less than or equal to 500/L. In some embodiments, the subject has a neutrophil count reduction of (c) less than or equal to 35%.

In some embodiments, the method comprises continuing administering to the subject a therapeutically effective amount of Compound (1) or the pharmaceutically acceptable salt, wherein the subject has a hemoglobin count reduction of (a) less than 1 grade compared to a baseline hemoglobin grade, (b) less than or equal to 5.0 g/dL compared to a baseline hemoglobin count, and/or (c) less than or equal to 35% compared to a baseline hemoglobin count, and the hemoglobin count reduction is determined by a change in the baseline hemoglobin count determined within one week prior to administering Compound (1) or the pharmaceutically acceptable salt and a second hemoglobin count determined at least 2 weeks after administering. In some embodiments, the subject has a hemoglobin count reduction of (a) less than 1 grade. In some embodiments, the subject has a hemoglobin count reduction of (b) less than or equal to 5.0 g/dL. In some embodiments, the subject has a hemoglobin count reduction of (c) less than or equal to 35%.

In some embodiments, the method results in the subject exhibiting bone marrow fibrosis reduction, total symptom reduction, cytokine modulation, or a combination of two or more thereof. In some embodiments, the method results in the subject exhibiting bone marrow fibrosis reduction. In some embodiments, the method results in the subject exhibiting total symptom reduction. In some embodiments, the method results in the subject exhibiting cytokine modulation.

In some embodiments, the method results in the subject exhibiting normalization or increased platelet count, normalization or increased neutrophil count, normalization or increased hemoglobin count, transfusion improvement (including conversion into transfusion independence), or a combination of two or more thereof. In some embodiments, the method results in the subject exhibiting normalization or increased platelet count. In some embodiments, the method results in the subject exhibiting normalization or increased neutrophil count. In some embodiments, the method results in the subject exhibiting normalization or increased hemoglobin count. In some embodiments, the method results in the subject exhibiting transfusion improvement (including conversion into transfusion independence).

In some embodiments, provided herein is a method for treating a myeloproliferative neoplasm in a human subject in need thereof, comprising administering to the subject a therapeutically effective amount of Compound (1) or a pharmaceutically acceptable salt thereof, wherein: the subject has had one or more previous treatments comprising Compound (1) or the pharmaceutically acceptable salt and has a platelet count reduction of (a) less than 1 grade, (b) less than or equal to 25×10⁹/L, and/or (c) less than or equal to 35%, the platelet count reduction is determined by a change in the baseline platelet count determined within one week prior to administering Compound (1) or the pharmaceutically acceptable salt and a second platelet count determined at least 2 weeks after administering, and the method results in the subject exhibiting bone marrow fibrosis reduction, total symptom reduction, cytokine modulation, or a combination of two or more thereof. In some embodiments, the method results in the subject exhibiting bone marrow fibrosis reduction. In some embodiments, the method results in the subject exhibiting total symptom reduction. In some embodiments, the method results in the subject exhibiting cytokine modulation. In some embodiments, the method results in the subject exhibiting bone marrow fibrosis reduction, total symptom reduction, and cytokine modulation.

In some embodiments, the subject has not been treated with a JAK inhibitor for at least 2 weeks prior to administering Compound (1) or the pharmaceutically acceptable salt.

In some embodiments, the total symptom reduction is a symptom selected from the group consisting of fever, night sweats, early satiety, weight loss, fatigue, weakness, shortness of breath, enlarged spleen or liver, left upper abdominal pain, bone or joint pain, easy bruising, easy bleeding, and combinations of two or more thereof.

In some embodiments, the total symptom reduction refers to minimizing, lessening, or ameliorating fever, night sweats, early satiety, weight loss, fatigue, weakness, shortness of breath, enlarged spleen or liver, left upper abdominal pain, bone or joint pain, easy bruising, easy bleeding, or a combination of two or more thereof.

In some embodiments, the method results in the subject exhibiting spleen volume reduction.

In some embodiments, the method results in bone marrow fibrosis reduction.

In some embodiments, the method results in improving overall survival.

In some embodiments, the method results in the subject exhibiting normalization or increased platelet count, normalization or increased neutrophil count, normalization or increased hemoglobin count, transfusion improvement (including conversion into transfusion independence), or a combination of two or more thereof.

In some embodiments, the methods of the current disclosure result in a durable response based on one or more measures of efficacy described herein. A durable response can be defined based on satisfaction of the efficacy measure at various timepoints, including 12 weeks, 24 weeks, 48 weeks, 72 weeks, or 96 weeks.

In some embodiments, when Compound (1) is co-administered with ruxolitinib or momelotinib, the therapeutic effective amounts of the drugs does not induce any overlapping hematological toxicities. I.e., Compound (1) does not exacerbate the anemia, thrombocytopenia, or transfusion dependence induced by the ruxolitinib or momelotinib.

Other embodiments are based on the probability of achieving certain therapeutic endpoints. Thus, in one embodiment the method induces a probability of SVR35 in the subject≥15% or 20%, wherein the subject has previously been treated by a JAK inhibitor for the myeloproliferative neoplasm and optionally has relapsed disease or is refractory to the JAK inhibitor. In another embodiment the method induces a probability of TSS50 in the subject≥30%, 35%, 40%, 45%, or 50%, wherein the subject has previously been treated by a JAK inhibitor for the myeloproliferative neoplasm and optionally has relapsed disease or is refractory to the JAK inhibitor.

In some embodiments, the subject has had an inadequate response to JAK inhibitor therapy.

In some embodiments, the subject is intolerant of a JAK inhibitor therapy, is resistant to a JAK inhibitor therapy, or is ineligible for a JAK inhibitor therapy.

In some embodiments, the subject experiences relapse from a prior JAK inhibitor therapy.

In some embodiments, the subject experiences relapse from a prior bone marrow transplant.

In some embodiments, the method further comprises administering to the subject an effective amount of a JAK inhibitor and/or an ACVR inhibitor or a pharmaceutically acceptable salt thereof (i.e., the PIM kinase inhibitor is administered concurrently with or sequentially to the JAK inhibitor and/or an ACVR inhibitor or a pharmaceutically acceptable salt thereof). In some embodiments, the JAK inhibitor is a JAK2 inhibitor. In some embodiments, the JAK inhibitor is a JAK1 inhibitor.

In some specific embodiments, the myeloproliferative neoplasm is polycythemia vera. In some other specific embodiments, the myeloproliferative neoplasm is essential thrombocythemia. In still other embodiments, the myeloproliferative neoplasm is MF. The structure of the PIM kinase inhibitor, the JAK inhibitor, and/or an ACVR inhibitor are not particularly limited provided the inhibitor has satisfactory activity against the desired target (i.e., PIM and JAK, respectively). Exemplary PIM kinase inhibitors which are included within the scope of embodiments of the present disclosure include the generic and specific compounds disclosed in PCT Pub. No. WO 2016/161248; WO 2015/019320; WO 2014/033530WO 2014/033631; WO 2014/0200216; WO 2013/175388; WO 2013/013188; WO 2013/020371; WO 2012/154274; WO 2012/129338; WO 2012/080990; WO 2012/120415; WO 2012/004217; WO 2011/057784; WO 2011/079274; WO 2010/0148351; WO 2010/135581; WO 2010/026121; WO 2010/026122; WO 2010/026124; WO 2010/022076; WO 2010/0000978; WO 2010/022081; WO 2009/064486; WO 2009/109576; WO 2008/082839; WO 2008/106692; WO 2008/058126; WO 2007/041712, U.S. Pat. Nos. 7,750,007; 8,168,794 and U.S. Pub. No. 2015/0057265; 2014/0200227; 2014/0329807, US 2008/0261988, the full disclosures of which are hereby incorporated by reference. In some embodiments, the PIM kinase inhibitor is PIM447 or INCB053914. Other PIM kinase inhibitors are known in the art, and such inhibitors are also included in certain embodiments of the disclosure.

The structure of the JAK inhibitor and/or an ACVR inhibitor for use in the present methods is also not particularly limited provided it has sufficient activity against JAK when used in combination with the PIM inhibitor. In some embodiments, the JAK inhibitor has sufficient activity against JAK2, when used in combination with the PIM inhibitor. In some embodiments, the JAK inhibitor has sufficient activity against JAK1, when used in combination with the PIM inhibitor. Exemplary JAK inhibitors, all of which are included within the scope of certain embodiments of the disclosure, are described in PCT Pub. Nos: WO 2015/157257; WO 2014/151871; WO 2014/026595; WO 2014/025128; WO 2014/025486; WO 2014/130411; WO 2014/124230; WO 2011/101161; WO 2011/076519; WO 2010/071885; WO 2010/017122; WO 2009/080638; WO 2009/143389; WO 2009/158571; WO 2009/017954; WO 2009/085913; WO 2009/155565; WO 2008/106635; WO 2008/128072; WO 2008/092199; WO 2005/026026130; WO 2004/046118WO 2004/074244; WO 2001/060816; 97/019065 and in U.S. Pub. Nos: 2015/0306112; 2013/0018034; 2012/0053208; 2008/0260754; and 2008/0214558, the full disclosures of which are hereby incorporated by reference.

Additional therapeutic agents may be used in combination with a PIM kinase inhibitor (i.e., Compound (1) or a pharmaceutically acceptable salt thereof) for treatment of a myeloproliferative neoplasm according to embodiments of the disclosure. Combinations of additional therapeutic agents can be administered simultaneously (e.g., in the same or different formulation) or sequentially with the PIM kinase inhibitor. For example, a PIM kinase inhibitor (i.e., Compound (1) or a pharmaceutically acceptable salt thereof) can be administered before a JAK inhibitor or before an ACVR inhibitor. Alternatively, PIM kinase inhibitor (i.e., Compound (1) or a pharmaceutically acceptable salt thereof) can be administered after a JAK inhibitor or after an ACVR inhibitor. In some of these embodiments, the JAK or ACVR inhibitor can be optionally administered in combination with the PIM kinase inhibitor and the additional therapeutic agent(s). That is, in some embodiments, the method comprises administering the PIM kinase inhibitor and an additional therapeutic agent.

In some embodiments, additional therapeutic agents include hydroxyurea, interferon alpha, cladribine, thalidomide (including derivatives thereof, e.g., pomalidomide, lenolidamide), corticosteroids (e.g., prednisone), everolimus, androgens (e.g., testosterone) and combinations thereof. In some embodiments, the additional therapeutic agents may be used for supportive care, such as corticosteroids, non-steroidal anti-inflammatory drugs, and/or analgesics to control symptoms.

In addition, the methods described herein can be performed in conjunction with other medical procedures. Accordingly, in some embodiments, the method further comprises performing a transfusion, administering radiation therapy, performing a splenectomy, or performing a stem cell transplant. In some specific embodiments, the method further comprises administering an angiotensin mimetic (e.g., TXA127).

Various different myeloproliferative neoplasms (MPN) can be treated by the methods disclosed herein. In some embodiments the MPN is polycythemia vera. In some embodiments, the MPN is essential thrombocythemia. In other embodiments, the MPN is MF.

Some embodiments provide a method for decreasing proliferation of hematopoietic cells in a subject, the method comprising contacting the cells with a PIM kinase inhibitor. In some more specific embodiments, the method further comprises administering to the subject an effective amount of an ACVR inhibitor. In some more specific embodiments, the method further comprises administering to the subject an effective amount of an ACVR1 inhibitor. In some more specific embodiments, the method further comprises administering to the subject an effective amount of a JAK inhibitor. In some embodiments, the JAK inhibitor is a JAK2, or JAK1, or JAK1/2 inhibitor.

In some embodiments, the disclosure is directed at a method of restoring hematological balance. In some different embodiments, the disclosure is directed to a method for decreasing proliferation of cells expressing JAK2 V617F in a human cell, the method comprising contacting the cell with a PIM kinase inhibitor as disclosed herein and optionally a JAK inhibitor as disclosed herein. In some different embodiments, the invention is directed to a method for decreasing proliferation of cells expressing MPL W515L in a human cell, the method comprising contacting the cell with a PIM kinase inhibitor as disclosed herein and optionally a JAK inhibitor as disclosed herein. In some different embodiments, the technology is directed to a method for decreasing proliferation of cells expressing a calreticulin (CALR) mutation in a human cell, the method comprising contacting the cell with a PIM kinase inhibitor as disclosed herein and optionally a JAK inhibitor or an ACVR as disclosed herein. In some different embodiments, the disclosure is directed to a method for decreasing proliferation or overproduction of red blood cells, white blood cells, or platelets in a human cell, the method comprising contacting the cell with a PIM kinase inhibitor as disclosed herein and optionally a JAK inhibitor or an ACVR as disclosed herein. In some embodiments, the human cell is a bone marrow cell.

In some specific embodiments, the myeloproliferative neoplasm of the subject treated for a myeloproliferative neoplasm according to the embodiments described herein comprises a JAK2 mutation, a thrombopoietin receptor (MPL) mutation, or a calreticulin (CALR) mutation. In some embodiments, the MPN comprises a JAK2 and an MPL mutation. In some embodiments, the MPN comprises a JAK2 and a CALR mutation. In some embodiments, the MPN comprises a MPL and a CALR mutation. In some embodiments the MPN comprises a JAK2, MPL, and CALR mutation.

In some embodiments, a JAK2 mutation comprises a JAK2 V617 mutation. JAK2 V617F refers to a mutated JAK2 possessing a V→F amino acid substitution at position 617 with respect to the human, wildtype JAK2 (UniProt. 060674). In some embodiments, a MPL mutation comprises a MPL W515L mutation. MPL W515L refers to a mutated thrombopoietin receptor (MPL) possessing a W→L substitution at position 515 with respect to the human, wildtype MPL (UniProt. P40238). In some embodiments, the mutation in CALR comprises a CALR exon 9 indel.

In some embodiments, provided herein is a method for treating a myeloproliferative neoplasm in a human subject in need thereof, comprising administering to the subject a therapeutically effective amount of Compound (1) or a pharmaceutically acceptable salt thereof, and ruxolitinib, fedratinib, pacritinib, momelotinib, luspatercept, ropeginterferon alfa-2b, or navtemadelin, or a pharmaceutically acceptable salt thereof, wherein: the subject has a baseline platelet count of less than or equal to 200×10⁹/L. Ruxolitinib or the pharmaceutically acceptable salt is commonly administered in a dose of about 5 mg to about 25 mg. The fedratinib or the pharmaceutically acceptable salt is in a dose of about 200 mg to about 500 mg; pacritinib or the pharmaceutically acceptable salt is in a dose of about 100 mg to about 200 mg; momelotinib or the pharmaceutically acceptable salt is in a dose of about 50 mg to about 200 mg; luspatercept is in a dose of about 0.5 mg/kg to 1.75 mg/kg based on the subject's body weight, ropeginterferon alfa-2b or the pharmaceutically acceptable salt is in a dose of 50 to 500 μg, and navtemadelin or the pharmaceutically acceptable salt is in a dose of 120 to 240 mg.

In some embodiments, provided herein is a method for treating a myeloproliferative neoplasm in a human subject in need thereof, comprising administering to the subject a therapeutically effective amount of Compound (1) or a pharmaceutically acceptable salt thereof, and ruxolitinib, fedratinib, pacritinib, momelotinib, luspatercept, ropeginterferon alfa-2b, or navtemadelin, or the pharmaceutically acceptable salt; wherein: the subject has a baseline platelet count of greater than or equal to 25×10⁹/L. The ruxolitinib or the pharmaceutically acceptable salt is commonly administered in a dose of about 5 mg to about 25 mg. The fedratinib or the pharmaceutically acceptable salt is in a dose of about 200 mg to about 500 mg; pacritinib or the pharmaceutically acceptable salt is in a dose of about 100 mg to about 200 mg; momelotinib or the pharmaceutically acceptable salt is in a dose of about 50 mg to about 200 mg; luspatercept is in a dose of about 0.6 mg/kg or 1.75 mg/kg based on the subject's body weight; ropeginterferon alfa-2b or the pharmaceutically acceptable salt is in a dose of 50 to 500 g; and navtemadelin or the pharmaceutically acceptable salt is in a dose of 120 to 240 mg.

In some embodiments, the baseline platelet count in the combination treatment is less than or equal to about 150×10⁹/L. In some embodiments, the baseline platelet count in the combination treatment is less than or equal to about 100×10⁹/L. In some embodiments, the baseline platelet count in the combination treatment is less than or equal to about 75×10⁹/L. In some embodiments, the baseline platelet count in the combination treatment is less than or equal to about 50×10⁹/L. In some embodiments, the baseline platelet count in the combination treatment is greater than or equal to about 10×10⁹/L. In some embodiments, the baseline platelet count in the combination treatment is greater than or equal to about 25×10⁹/L. In some embodiments, the baseline platelet count in the combination treatment is greater than or equal to about 35×10⁹/L.

In some embodiments, provided herein is a method for treating a myeloproliferative neoplasm in a human subject in need thereof, comprising administering to the subject a therapeutically effective amount of Compound (1) or a pharmaceutically acceptable salt thereof, and ruxolitinib or a pharmaceutically acceptable salt thereof. The subject has a baseline platelet count of less than or equal to 200×10⁹/L; and ruxolitinib or the pharmaceutically acceptable salt is in a dose of about 5 mg to 25 mg.

In some embodiments, provided herein is a method for treating a myeloproliferative neoplasm in a human subject in need thereof, comprising administering to the subject a therapeutically effective amount of Compound (1) or a pharmaceutically acceptable salt thereof, and fedratinib or a pharmaceutically acceptable salt thereof. The subject has a baseline platelet count of less than or equal to 200×10⁹/L and/or a baseline platelet count of greater than or equal to 25×10⁹/L; and fedratinib or the pharmaceutically acceptable salt is in a dose of about 200 mg to about 500 mg.

In some embodiments, provided herein is a method for treating a myeloproliferative neoplasm in a human subject in need thereof, comprising administering to the subject a therapeutically effective amount of Compound (1) or a pharmaceutically acceptable salt thereof, and pacritinib or a pharmaceutically acceptable salt thereof. The subject has a baseline platelet count of less than or equal to 200×10⁹/L and/or a baseline platelet count of greater than or equal to 25×10⁹/L; and pacritinib or the pharmaceutically acceptable salt is in a dose of about 100 mg to about 200 mg.

In some embodiments, provided herein method for treating a myeloproliferative neoplasm in a human subject in need thereof, comprising administering to the subject a therapeutically effective amount of Compound (1) or a pharmaceutically acceptable salt thereof; and momelotinib or a pharmaceutically acceptable salt thereof. The subject has a baseline platelet count of less than or equal to 200×10⁹/L and/or a baseline platelet count of greater than or equal to 25×10⁹/L; and momelotinib or the pharmaceutically acceptable salt is in a dose of about 50 mg to about 200 mg.

In some embodiments, provided herein is a method for treating a myeloproliferative neoplasm in a human subject in need thereof, comprising administering to the subject a therapeutically effective amount of Compound (1) or a pharmaceutically acceptable salt thereof, and luspatercept. The subject has a baseline platelet count of less than or equal to 200×10⁹/L and/or a baseline platelet count of greater than or equal to 25×10⁹/L; and luspatercept is in a dose of about 0.5 mg/kg or 1.75 mg/kg based on the subject's body weight.

In some embodiments, the baseline platelet count is measured within one week prior to initiation of the administering Compound (1) or the pharmaceutically acceptable salt.

In some embodiments of the combination treatments, the subject has not been treated with any JAK inhibitor therapy for at least 2 weeks prior to administering Compound (1) or the pharmaceutically acceptable salt.

In some embodiments, provided herein is a method of treating a myeloproliferative neoplasm in a human subject in need thereof, without causing a clinically significant risk of adverse events, comprising: administering to the subject a dose from about 5 mg to about 25 mg of ruxolitinib or a pharmaceutically acceptable salt thereof for a period of up to about 36 months; determining or having determined if the subject had an adverse event during the treatment period; reducing the dose amount from about 33% to about 80%, or temporarily stopping the dose; and administering to the subject a therapeutically effective amount of Compound (1) or a pharmaceutically acceptable salt thereof.

In some embodiments, the adverse event in the combination treatment of Compound (1) or the pharmaceutically acceptable salt and ruxolitinib is thrombocytopenia, anemia, bruising, dizziness, headache, diarrhea, or a combination of two or more thereof.

In some embodiments, provided herein is a method of treating a myeloproliferative neoplasm in a human subject in need thereof while the subject is undergoing a ruxolitinib therapy comprising about 5 mg to about 25 mg of ruxolitinib or a pharmaceutically acceptable salt thereof, the method comprises: administering to the subject a therapeutically effective amount of Compound (1) or a pharmaceutically acceptable salt thereof; continuing to administer to the subject the therapeutically effective amount of Compound (1) or the pharmaceutically acceptable salt, during which the subject either (a) does not receive the therapy (e.g., JAK therapy such as ruxolitinib), or (b) receives a reduced amount of the therapy (e.g., JAK therapy such as ruxolitinib).

In some embodiments, the reduced amount of the therapy comprises less than about 80% amount of the therapy or the pharmaceutically acceptable salt. In some embodiments, the reduced amount of the therapy comprises less than about 75% amount of the therapy or the pharmaceutically acceptable salt. In some embodiments, the reduced amount of the therapy comprises less than about 50% amount of the therapy or the pharmaceutically acceptable salt. In some embodiments, the reduced amount of the therapy comprises less than about 25% amount of the therapy or the pharmaceutically acceptable salt. In some embodiments, the reduced amount of the therapy comprises less than about 20% amount of the therapy or the pharmaceutically acceptable salt. In some embodiments, the reduced amount of the therapy comprises about 10% to about 80% amount of the therapy or the pharmaceutically acceptable salt. In some embodiments, the reduced amount of the therapy comprises about 30% to about 70% amount of the therapy or the pharmaceutically acceptable salt. In some embodiments, the reduced amount of the therapy comprises about 40% to about 60% amount of the therapy or the pharmaceutically acceptable salt.

In some embodiments, provided herein is a method of treating a myeloproliferative neoplasm in a human subject in need thereof, without causing a clinically significant risk of adverse events, comprising: administering to the subject a dose from about 200 mg to about 500 mg of fedratinib or a pharmaceutically acceptable salt thereof for a period of up to about 36 months; determining or having determined if the subject had an adverse event during the treatment period; reducing the dose amount from about 33% to about 80%, or temporarily stopping the dose; and administering to the subject a therapeutically effective amount of Compound (1) or a pharmaceutically acceptable salt thereof.

In some embodiments, the adverse event in the combination treatment of Compound (1) or the pharmaceutically acceptable salt and fedratinib is thrombocytopenia, anemia diarrhea, nausea, vomiting, encephalopathy, or a combination of two or more thereof.

In some embodiments, the reduced amount of the fedratinib therapy comprises ≤50% or 75% amount of fedratinib or the pharmaceutically acceptable salt.

In some embodiments, provided herein is a method of treating a myeloproliferative neoplasm in a human subject in need thereof, without causing a clinically significant risk of adverse events, comprising: administering to the subject a dose from about 100 mg to about 200 mg of pacritinib or a pharmaceutically acceptable salt thereof for a period of up to about 36 months; determining or having determined if the subject had an adverse event during the treatment period; reducing the dose amount from about 33% to about 80%, or temporarily stopping the dose; and administering to the subject a therapeutically effective amount of Compound (1) or a pharmaceutically acceptable salt thereof.

In some embodiments, the adverse event in the combination treatment of Compound (1) or the pharmaceutically acceptable salt and pacritinib is thrombocytopenia, anemia diarrhea, nausea, peripheral edema, or a combination of two or more thereof.

In some embodiments, the reduced amount of the pacritinib therapy comprises ≤50% or 75% amount of pacritinib or the pharmaceutically acceptable salt.

In some embodiments, provided herein is a method of treating a myeloproliferative neoplasm in a human subject in need thereof, without causing a clinically significant risk of adverse events, comprising: administering to the subject a dose from about 50 mg to about 200 mg of momelotinib or a pharmaceutically acceptable salt thereof for a period of up to about 36 months; determining or having determined if the subject had an adverse event during the treatment period; reducing the dose amount from about 33% to about 80%, or temporarily stopping the dose; and administering to the subject a therapeutically effective amount of Compound (1) or a pharmaceutically acceptable salt thereof.

In some embodiments, the adverse event in the combination treatment of Compound (1) or the pharmaceutically acceptable salt and momelotinib is thrombocytopenia, anemia diarrhea, nausea, dizziness, or a combination of two or more thereof.

In some embodiments, provided herein is a method of treating a myeloproliferative neoplasm in a human subject in need thereof while the subject is undergoing a momelotinib therapy comprising from about 50 mg to about 200 mg of momelotinib or a pharmaceutically acceptable salt thereof, wherein the method comprises: administering to the subject a therapeutically effective amount of Compound (1) or a pharmaceutically acceptable salt thereof; continuing to administer to the subject the therapeutically effective amount of Compound (1) or the pharmaceutically acceptable salt, during which the subject either (a) does not receive the momelotinib therapy, or (b) receives a reduced amount of the momelotinib therapy.

In some embodiments, the subject receives a reduced amount of the momelotinib therapy. In some embodiments, the reduced amount of the momelotinib therapy comprises ≤50% or 75% amount of momelotinib or the pharmaceutically acceptable salt.

In some embodiments, provided herein is a method of treating a myeloproliferative neoplasm in a human subject in need thereof, without causing a clinically significant risk of adverse events, comprising: administering to the subject a dose from about 0.6 mg/kg or 1.75 mg/kg of luspatercept for a period of up to about 36 months; determining or having determined if the subject had an adverse event during the treatment period; reducing the dose amount from about 33% to about 80%, or temporarily stopping the dose; and administering to the subject a therapeutically effective amount of Compound (1) or a pharmaceutically acceptable salt thereof.

In some embodiments, the adverse event in the combination treatment of Compound (1) or the pharmaceutically acceptable salt and luspatercept is fatigue, headache, musculoskeletal pain, arthralgia, dizziness/vertigo, nausea, diarrhea, cough, abdominal pain, dyspnea, hypersensitivity, or a combination of two or more thereof.

In some embodiments, provided herein is a method of treating a myeloproliferative neoplasm in a human subject in need thereof while the subject is undergoing a luspatercept therapy comprising from about 0.5 mg/kg or 1.75 mg/kg of luspatercept, wherein the method comprises: administering to the subject a therapeutically effective amount of Compound (1) or a pharmaceutically acceptable salt thereof; continuing to administer to the subject the therapeutically effective amount of Compound (1) or the pharmaceutically acceptable salt, during which the subject either (a) does not receive the luspatercept therapy, or (b) receives a reduced amount of the luspatercept therapy.

In some embodiments, the reduced amount of the luspatercept therapy comprises ≤50% or 75% amount of luspatercept.

In some embodiments, administration of the combination minimizes, lessens, or ameliorates adverse events in the subject.

In some embodiments, administration of the combination reduces bone marrow fibrosis in the subject.

In some embodiments, administration of the combination reduces spleen volume in the subject.

In some embodiments, administration of the combination results in the subject exhibiting normalization or increased platelet count, normalization or increased neutrophil count, normalization or increased hemoglobin count, transfusion improvement (including conversion into transfusion independence), or a combination of two or more thereof.

In some embodiments, administration of the combination results in the subject exhibiting normalization or increased platelet count.

In some embodiments, administration of the combination results in the subject exhibiting normalization or increased neutrophil count.

In some embodiments, administration of the combination results in the subject exhibiting normalization or increased hemoglobin count.

In some embodiments, administration of the combination results in the subject exhibiting transfusion improvement (including conversion into transfusion independence).

In some embodiments, the therapeutically effective amount of Compound (1) or the pharmaceutically acceptable salt is from about 120 mg to about 1500 mg.

In some embodiments, the therapeutically effective amount of Compound (1) or the pharmaceutically acceptable salt is from about 240 mg to about 1440 mg daily. In some embodiments, the therapeutically effective amount of Compound (1) or the pharmaceutically acceptable salt is from about 240 mg to about 1000 mg. In some embodiments, the therapeutically effective amount of Compound (1) or the pharmaceutically acceptable salt is from about 240 mg to about 900 mg. In some embodiments, the therapeutically effective amount of Compound (1) or the pharmaceutically acceptable salt is from about 240 mg to about 800 mg. In some embodiments, the therapeutically effective amount of Compound (1) or the pharmaceutically acceptable salt is from about 240 mg to about 700 mg. In some embodiments, the therapeutically effective amount of Compound (1) or the pharmaceutically acceptable salt is from about 240 mg to about 600 mg. In some embodiments, the therapeutically effective amount of Compound (1) or the pharmaceutically acceptable salt is from about 240 mg to about 500 mg.

In some embodiments, the therapeutically effective amount of Compound (1) or the pharmaceutically acceptable salt is from about 350 mg to about 1350 mg. In some embodiments, the therapeutically effective amount of Compound (1) or the pharmaceutically acceptable salt is from about 400 mg to about 1150 mg. In some embodiments, the therapeutically effective amount of Compound (1) or the pharmaceutically acceptable salt is from about 500 mg to about 1050 mg.

In some embodiments, the therapeutically effective amount of Compound (1) or the pharmaceutically acceptable salt is from about 350 mg to about 1000 mg. In some embodiments, the therapeutically effective amount of Compound (1) or the pharmaceutically acceptable salt is from about 450 mg to about 900 mg. In some embodiments, the therapeutically effective amount of Compound (1) or the pharmaceutically acceptable salt is from about 450 mg to about 800 mg. In some embodiments, the therapeutically effective amount of Compound (1) or the pharmaceutically acceptable salt is from about 450 mg to about 750 mg. In some embodiments, the therapeutically effective amount of Compound (1) or the pharmaceutically acceptable salt is from about 470 mg to about 730 mg.

In some embodiments, the therapeutically effective amount of Compound (1) or the pharmaceutically acceptable salt is from about 120 mg to about 580 mg, about 581 mg to about 1040 mg, or about 1041 mg to about 1500 mg.

In some embodiments, administering the therapeutically effective amount of Compound (1) or the pharmaceutically acceptable salt is at least once daily. In some embodiments, administering the therapeutically effective amount of Compound (1) or the pharmaceutically acceptable salt is at least twice daily. In some embodiments, administering the therapeutically effective amount of Compound (1) or the pharmaceutically acceptable salt is at least three times daily. In some embodiments, administering the therapeutically effective amount of Compound (1) or the pharmaceutically acceptable salt is at least four times daily.

In some embodiments, the therapeutically effective amount of Compound (1) or the pharmaceutically acceptable salt is from about 120 mg/day to about 3000 mg/day. In some embodiments, the therapeutically effective amount of Compound (1) or the pharmaceutically acceptable salt is from about 350 mg/day to about 2100 mg/day. In some embodiments, the therapeutically effective amount of Compound (1) or the pharmaceutically acceptable salt is from about 550 mg/day to about 2000 mg/day. In some embodiments, the therapeutically effective amount of Compound (1) or the pharmaceutically acceptable salt is from about 700 mg/day to about 1950 mg/day.

In some embodiments, the therapeutically effective amount of Compound (1) or the pharmaceutically acceptable salt is from about 120 mg/day to about 800 mg/day. In some embodiments, the therapeutically effective amount of Compound (1) or the pharmaceutically acceptable salt is from about 200 mg/day to about 750 mg/day. In some embodiments, the therapeutically effective amount of Compound (1) or the pharmaceutically acceptable salt is from about 250 mg/day to about 650 mg/day.

In some embodiments, the therapeutically effective amount of Compound (1) or the pharmaceutically acceptable salt is from about 240 mg to about 1440 mg twice daily. In some embodiments, the therapeutically effective amount of Compound (1) or the pharmaceutically acceptable salt is from about 360 mg to about 1440 mg twice daily. In some embodiments, the therapeutically effective amount of Compound (1) or the pharmaceutically acceptable salt is about 1440 mg twice daily.

In some embodiments, ruxolitinib or the pharmaceutically acceptable salt is about 2.5 mg, 5 mg, 7.5 mg, 10 mg, 12.5 mg, 15 mg, 20 mg, or 25 mg. In some embodiments, the dose amount of ruxolitinib or the pharmaceutically acceptable salt may be about 2.5 mg. In some embodiments, the dose amount of ruxolitinib or the pharmaceutically acceptable salt may be about 5 mg. In some embodiments, the dose amount of ruxolitinib or the pharmaceutically acceptable salt may be about 7.5 mg. In some embodiments, the dose amount of ruxolitinib or the pharmaceutically acceptable salt may be about 10 mg. In some embodiments, the dose amount of ruxolitinib or the pharmaceutically acceptable salt may be about 12.5 mg. In some embodiments, the dose amount of ruxolitinib or the pharmaceutically acceptable salt may be about 15 mg. In some embodiments, the dose amount of ruxolitinib or the pharmaceutically acceptable salt may be about 20 mg. In some embodiments, the dose amount of ruxolitinib or the pharmaceutically acceptable salt may be about 25 mg.

In some embodiments, the dose amount of fedratinib or the pharmaceutically acceptable salt may be about 100 mg. In some embodiments, the dose amount of fedratinib or the pharmaceutically acceptable salt may be about 200 mg. In some embodiments, the dose amount of fedratinib or the pharmaceutically acceptable salt may be about 400 mg. In some embodiments, the dose amount of fedratinib or the pharmaceutically acceptable salt may be about 500 mg.

In some embodiments, the dose amount of pacritinib or the pharmaceutically acceptable salt may be about 50 mg. In some embodiments, the dose amount of pacritinib or the pharmaceutically acceptable salt may be about 100 mg. In some embodiments, the dose amount of pacritinib or the pharmaceutically acceptable salt may be about 200 mg.

In some embodiments, the dose amount of momelotinib or the pharmaceutically acceptable salt may be about 25 mg. In some embodiments, the dose amount of momelotinib or the pharmaceutically acceptable salt may be about 50 mg. In some embodiments, the dose amount of momelotinib or the pharmaceutically acceptable salt may be about 100 mg. In some embodiments, the dose amount of momelotinib or the pharmaceutically acceptable salt may be about 200 mg.

In some embodiments, the dose amount of luspatercept may be about 0.6 mg/kg based on the subject's body weight. In some embodiments, the dose amount of luspatercept may be about 0.8 mg/kg based on the subject's body weight. In some embodiments, the dose amount of luspatercept may be about 1.0 mg/kg based on the subject's body weight. In some embodiments, the dose amount of luspatercept may be about 1.25 mg/kg based on the subject's body weight. In some embodiments, the dose amount of luspatercept may be about 1.33 mg/kg based on the subject's body weight. In some embodiments, the dose amount of luspatercept may be about 1.50 mg/kg based on the subject's body weight. In some embodiments, the dose amount of luspatercept may be about 1.75 mg/kg based on the subject's body weight. In some embodiments, luspatercept is administrated once every 3 weeks by subcutaneous injection for at least 21 weeks.

In some embodiments, administering ruxolitinib, fedratinib, pacritinib, or momelotinib, or the pharmaceutically acceptable salt is at least one dose a day. In some embodiments, administering ruxolitinib, fedratinib, pacritinib, or momelotinib, or the pharmaceutically acceptable salt is at least two doses a day.

In some embodiments, administering Compound (1) or the pharmaceutically acceptable salt and ruxolitinib, fedratinib, pacritinib, or momelotinib, or the pharmaceutically acceptable salt thereof is concurrent.

In some embodiments, administering Compound (1) or the pharmaceutically acceptable salt and ruxolitinib, fedratinib, pacritinib, momelotinib, luspatercept, ropeginterferon alfa-2b, or navtemadelin, or the pharmaceutically acceptable salt is sequential.

In some embodiments, the myeloproliferative neoplasm is a JAK inhibitor-resistant myeloproliferative neoplasm.

In some embodiments, the subject has been previously treated with a JAK inhibitor and exhibited an inadequate response.

In some embodiments, the inadequate response is resistance or intolerance to the JAK inhibitor, loss or failure to obtain significant spleen response or total symptom response, and/or developing clinically significant reduction of count of blood cells, neutropenia, or thrombocytopenia.

In some embodiments, the subject has not been previously treated with a JAK inhibitor.

In some embodiments, the present technology further comprises monitoring complete blood counts in the subject and titrating the dose amount of Compound (1) or a pharmaceutically acceptable salt thereof.

In some embodiments, the monitoring comprises assessing platelet counts and increasing the dose amount or frequency (i.e., up-titrating) if the platelet count change is (a) less than 1 grade, (b) decreased by less than 25×10⁹/L, and/or (c) decreased by less than 35%, based on the subject's baseline platelet count (i.e., platelet count prior to starting treatment).

In some embodiments, the monitoring comprises assessing platelet counts and decreasing the dose amount or less frequency (i.e., down-titrating) if the platelet count change is (a) up 1 or more grades, (b) decreased by more than 25×10⁹/L, and/or (c) decreased by more than 35%.

In some embodiments, the platelet count has moved up from grade 1 to grade 2, 3, or 4, or from grade 2 to grade 3 or 4, or from grade 3 to grade 4.

In some embodiments, the monitoring comprises assessing neutrophil counts and decreasing the dose amount or less frequency (i.e., down-titrating) if the neutrophil count change is (a) up 1 or more grades, (b) decreased by more than 500/L, and/or (c) decreased by more than 35%.

In some embodiments, the neutrophil count has moved up from grade 1 to grade 2, 3, or 4, or from grade 2 to grade 3 or 4, or from grade 3 to grade 4.

In some embodiments, the monitoring comprises assessing hemoglobin counts and decreasing the dose amount or less frequency (i.e., down-titrating) if the hemoglobin count change is (a) up 1 or more grades, (b) decreased by more than 5.0 g/dL, and/or (c) decreased by more than 35%.

In some embodiments, the hemoglobin count has moved up from grade 1 to grade 2 or 3, or from grade 2 to grade 3.

In some embodiments, the monitoring is performed prior to, during, and/or after the treatment.

In some embodiments, the monitoring is performed once a week or every 2 to 4 weeks. In some embodiments, the monitoring is performed once a week. In some embodiments, the monitoring is performed every other week. In some embodiments, the monitoring is performed every 3 weeks. In some embodiments, the monitoring is performed every 4 weeks.

In some embodiments, the monitoring further comprises continuing the titrating the dose amount or frequency until achieving a desired stable clinical state.

In some embodiments, the method further comprises monitoring complete blood counts in the subject and titrating the dose amount of ruxolitinib, fedratinib, pacritinib, momelotinib, luspatercept, ropeginterferon alfa-2b, or navtemadelin, or a pharmaceutically acceptable salt thereof.

In some embodiments, the method further comprises monitoring complete blood counts in the subject and titrating the dose amount of ruxolitinib or a pharmaceutically acceptable salt thereof.

In some embodiments, the method further comprises monitoring complete blood counts in the subject and titrating the dose amount of fedratinib or a pharmaceutically acceptable salt thereof.

In some embodiments, the method further comprises monitoring complete blood counts in the subject and titrating the dose amount of pacritinib or a pharmaceutically acceptable salt thereof.

In some embodiments, the method further comprises monitoring complete blood counts in the subject and titrating the dose amount of momelotinib or a pharmaceutically acceptable salt thereof.

In some embodiments, the method further comprises monitoring complete blood counts in the subject and titrating the dose amount of luspatercept.

In some embodiments, the method monitoring comprises assessing platelet counts in the subject prior to, during, and/or after the treatment.

In some embodiments, when administered in combination with Compound (1), titrating comprises starting ruxolitinib or a pharmaceutically acceptable salt thereof in the subject at a dose: 20 mg twice daily if baseline platelet count is greater than 200×10⁹/L, 10 mg twice daily if baseline platelet count is from 100×10⁹/L to 200×10⁹/L, 5 mg twice daily if baseline platelet count is from 50×10⁹/L to less than 100×10⁹/L.

In some embodiments, the method comprises increasing the dose amount of ruxolitinib if the subject's response is insufficient and platelet and neutrophil counts are adequate.

In some embodiments, when administered in combination with Compound (1), the method comprises increasing the dose of ruxolitinib or the pharmaceutically acceptable salt in the amount of 5 mg twice daily increments to a maximum of 25 mg twice daily.

In some embodiments, the method comprises increasing the dose amount of ruxolitinib or a pharmaceutically acceptable salt thereof in the subject that meets all of the following conditions: (a) failure to achieve a reduction from pretreatment baseline in either palpable spleen length of 50% or a 35% reduction in spleen volume as measured by computed tomography (CT) or magnetic resonance imaging (MRI); (b) platelet count greater than 125×10⁹/L at 4 weeks and platelet count never below 100×10⁹/L; and (c) ANC Levels greater than 0.75×10⁹/L.

In some embodiments, the method comprises increasing the dose amount of ruxolitinib or a pharmaceutically acceptable salt thereof in the subject by increments of 5 mg daily to a maximum of 10 mg twice daily if: a) the platelet count has remained at least 40×10⁹/L, b) the platelet count has not fallen by more than 20% in the prior 4 weeks, c) the ANC is more than 1×10⁹/L, and d) the dose has not been reduced or interrupted for an adverse event or hematological toxicity in the prior 4 weeks.

In some embodiments, titrating the dose amount of ruxolitinib or a pharmaceutically acceptable salt thereof is in according to Table (a) for thrombocytopenia for the subject whose starting treatment with a platelet count of 100×10⁹/L or greater:

TABLE (a)

| | Dose at Time of Platelet Decline | | | | |
|---|---|---|---|---|---|
| | 25 mg | 20 mg | 15 mg | 10 mg | 5 mg |
| | twice daily | twice daily | twice daily | twice daily | twice daily |
| Platelet Count | New Dose | New Dose | New Dose | New Dose | New Dose |
| 100 to less than | 20 mg | 15 mg | No Change | No Change | No Change |
| 125 × 10⁹/L | twice daily | twice daily | | | |
| 75 to less than | 10 mg | 10 mg | 10 mg | No Change | No Change |
| 100 × 10⁹/L | twice daily | twice daily | twice daily | | |
| 50 to less than | 5 mg | 5 mg | 5 mg | 5 mg | No Change |
| 75 × 10⁹/L | twice daily | twice daily | twice daily | twice daily | |
| Less than 50 × 10⁹/L | Hold | Hold | Hold | Hold | Hold |

In some embodiments, titrating the dose amount of ruxolitinib or a pharmaceutically acceptable salt thereof is in according to Table (b) for thrombocytopenia for the subject whose starting treatment with a platelet count of 50×10⁹/L to less than 100×10⁹/L:

TABLE (b)

| Platelet Count | Dosing Recommendations |
|---|---|
| Less than 25 × 10⁹/L | Interrupt dosing. |
| 25 × 10⁹/L to less than 35 × 10⁹/L | Decrease dose by 5 mg once daily. |
| AND the platelet count decline is | For patients on 5 mg once daily, |
| less than 20% during the prior | maintain dose at 5 mg once daily. |
| four weeks | |
| 25 × 10⁹/L to less than 35 × 10⁹/L | Decrease dose by 5 mg twice daily. |
| AND the platelet count decline is | For patients on 5 mg twice daily, |
| 20% or greater during the prior | decrease the dose to 5 mg once daily. |
| four weeks | For patients on 5 mg once daily, |
| | maintain dose at 5 mg once daily. |

In some embodiments, the method comprises starting fedratinib or a pharmaceutically acceptable salt thereof in the subject at 200, 300, or 400 mg once daily for patients with a baseline platelet count of greater than or equal to 50×10⁹/L.

In some embodiments, the method comprises reducing the dose amount of fedratinib to 100 mg or below, or temporarily stopping the doses if adverse reactions develop.

In some embodiments, the adverse reaction of the fedratinib therapy is anemia, grade 4 thrombocytopenia, grade 3 thrombocytopenia with active bleeding, grade 3 or higher nausea, vomiting, diarrhea, anemia, or elevations of alanine transaminase (ALT), aspartate aminotransferase (AST), or bilirubin.

In some embodiments, the method comprises starting pacritinib or a pharmaceutically acceptable salt thereof in the subject at 100 mg or 200 mg twice daily for patients with a baseline platelet count below 50×10⁹/L.

In some embodiments, the method comprises reducing the doses amount of pacritinib to 100 mg or below if adverse reactions develop.

In some embodiments, the adverse reaction of the pacritinib therapy is diarrhea, thrombocytopenia, nausea, anemia, and peripheral edema.

In some embodiments, the method further comprises temporarily stopping the dose of pacritinib if any clinically significant worsening of thrombocytopenia that last more than 7 days, until toxicity is resolved, and restarting pacritinib or the pharmaceutically acceptable salt at 50% of last dose given.

In some embodiments, the method comprises starting luspatercept in the subject at 0.5 mg/kg or 1 mg/kg by subcutaneous injection once every 3 weeks.

In some embodiments, titrating the dose amount of luspatercept if the subject's response is insufficient or adverse actions develop.

In some embodiments, further comprises titrating the dose of luspatercept based on responses according to Table (c):

TABLE (c)
Dose Increases for Insufficient Response at Initiation of Treatment

| | |
|---|---|
| No reduction in RBC transfusion burden after | Increase the dose to 1.25 |
| at least 2 consecutive doses (6 weeks) at | mg/kg every 3 weeks |
| the 1 mg/kg starting dose | |
| No reduction in RBC transfusion burden after | Discontinue treatment |
| 3 consecutive doses (9 weeks) at 1.25 mg/kg | |

In some embodiments, titrating the dose of luspatercept is based on responses according to Table (d):

TABLE (d)
Dose Modifications for Predose Hemoglobin
Levels or Rapid Hemoglobin Rise

| | |
|---|---|
| Predose hemoglobin is greater than or | Interrupt treatment |
| equal to 11.5 g/dL in the absence of | Restart when the hemoglobin |
| transfusions | is no more than 11 g/dL |
| Increase in hemoglobin greater than | |
| 2 g/dL within 3 weeks in the absence | |
| of transfusions and | |
| current dose is 1.25 mg/kg | Reduce dose to 1 mg/kg |
| current dose is 1 mg/kg | Reduce dose to 0.8 mg/kg |
| current dose is 0.8 mg/kg | Reduce dose to 0.6 mg/kg |
| current dose is 0.6 mg/kg | Discontinue treatment |

In some embodiments, the method of administering momelotinib or a pharmaceutically acceptable salt thereof in the subject at a dose of about 50 mg to about 200 mg when the subject is suffering from intermediate or high-risk primary or secondary (post-PV or post-ET) myelofibrosis with hemoglobin level<10.

In some embodiments, the method results in the subject exhibiting total symptom stability.

In some embodiments, the method results in the subject exhibiting total symptom reduction.

In some embodiments, the method results in the subject exhibiting partial symptom reduction.

In some embodiments, the method results in the subject exhibiting spleen volume reduction.

In some embodiments, the method results in the subject exhibiting normalization or increased platelet count, normalization or increased neutrophil count, normalization or increased hemoglobin count, transfusion improvement (including conversion into transfusion independence), or a combination of two or more thereof.

In some embodiments, method results in the subject being cytogenetic remission or molecular partial or complete remission.

In some embodiments, the method improves clinically meaningful overall survival in the subject.

In some embodiments, the method ameliorates anemia in the subject.

In some embodiments, the method results in ≥20 g/L increasing in hemoglobin level or changing transfusion-depending to transfusion-independent in the subject.

In some embodiments, the method results in improvement of Patient Global Impression of Change (PGIC) questionnaire (at least 1 score increase) at week 24.

In some embodiments, a myeloproliferative neoplasm is myelofibrosis.

In some embodiments, the myelofibrosis is intermediate-risk myelofibrosis.

In some embodiments, the myelofibrosis is high-risk myelofibrosis.

In some embodiments, the myelofibrosis is primary myelofibrosis.

In some embodiments, the myelofibrosis is secondary myelofibrosis. (e.g., post-PV myelofibrosis and post-ET myelofibrosis).

In some embodiments, the pharmaceutically acceptable salt thereof is a hydrochloric acid salt.

In some embodiments, the hydrochloric acid salt of Compound (1) is in a crystalline form.

In some embodiments, administering Compound (1) or the pharmaceutically acceptable salt is for a period from about one week to about two years.

In some embodiments, administering Compound (1) or the pharmaceutically acceptable salt is for a period from about 15 weeks to about one year.

In some embodiments, administering Compound (1) or the pharmaceutically acceptable salt is for from about 24 weeks to about one year.

In some embodiments, the subject with MF is stratified into a risk group using the Dynamic International Prognostic Scoring System (DIPSS), Mutation-enhanced International Prognostic Scoring System plus karyotype (MIPSS70+), or other systems. Risk factors using DIPSS include age, symptomatic burden (e.g., weight loss, fever, or excessive sweating), white blood cell counts, hemoglobin, and peripheral blasts. Patients having low-risk MF have a DIPSS score of 0. In some embodiments, the MF is low-risk MF.

Patients having intermediate-risk MF have a DIPSS score of 1 to 4. A DIPSS score of 1-2 is also referred to as intermediate-1 risk, and is typically associated with a median survival of about 14.2 years. A DIPSS score of 3 or 4 is also referred to as intermediate-2 risk, and is typically associated with a median survival of 4 years. In some embodiments, the MF is intermediate-risk MF (e.g., intermediate-1 risk MF, intermediate-2 risk MF).

Patients having high-risk MF have a DIPSS score of 5 or 6, associated with a median survival of 1.5 years. In some embodiments, the MF is high-risk MF.

In some embodiments, the MPN is a ruxolitinib-resistant MPN (e.g., ruxolitinib-resistant MF). In some embodiments, the MPN (e.g., MF) has been previously treated with ruxolitinib, e.g., in the absence of a PIM kinase inhibitor (i.e., Compound (1) or a pharmaceutically acceptable salt thereof).

In some embodiments, treating the MPN in accordance with the methods described herein results in complete remission in the subject. When used to refer to a human subject having an MPN, such as MF, “complete remission” means the subject meets the following criteria for 12 weeks: bone marrow shows age-adjusted normocellularity, <5% blasts and ≤grade 1 MF according to the European classification; and hemoglobin 100 g/L and <upper normal limit (UNL), and neutrophil count≥1×10⁹/L and <UNL in peripheral blood; and platelet count≥100×10⁹/L and <UNL, and <2% immature myeloid cells, except that in splenectomized patients, <5% immature myeloid cells is allowed; and resolution of disease symptoms (e.g., total symptoms), non-palpable spleen and liver, and no evidence of extramedullary hematopoiesis (EMH).

In some embodiments, treating the MPN in accordance with the methods described herein results in partial remission in the subject. When used to refer to a human subject having an MPN, such as MF, “partial remission” means the subject meets the following criteria for ≥12 weeks: hemoglobin≥100 g/L and <UNL, and neutrophil count≥1×10⁹/L and <UNL in peripheral blood; and platelet count≥100×10⁹/L and <UNL, and <2% immature myeloid cells, except that in splenectomized patients, <5% immature myeloid cells is allowed; and resolution of disease symptoms (e.g., total symptoms), non-palpable spleen and liver, and no evidence of extramedullary hematopoiesis (EMH). Alternatively, “partial remission” means bone marrow shows age-adjusted normocellularity, <5% blasts and ≤grade 1 MF according to the European classification; and hemoglobin≥85 but <100 g/L, and neutrophil count≥1×10⁹/L and <UNL in peripheral blood; and platelet count≥50 but <100×10⁹/L, and <2% immature myeloid cells, except that in splenectomized patients, <5% immature myeloid cells is allowed; and resolution of disease symptoms (e.g., total symptoms), non-palpable spleen and liver, and no evidence of extramedullary hematopoiesis (EMH).

In some embodiments, treating the MPN in accordance with the methods described herein results in the subject being cytogenetic complete remission, cytogenetic partial remission, molecular complete remission, or molecular partial remission. In some embodiments, treating the MPN in accordance with the methods described herein results in cytogenetic or molecular remission in the subject.

As used to herein, “cytogenetic complete remission” or “molecular complete remission” refers to the subject having eradication of pre-existing abnormality for ≥6 months.

As used herein, “cytogenetic partial remission” refers to the subject having ≥50% reduction in abnormal metaphases in patients with at least 10 abnormal metaphases at before treatment.

In some embodiments, the subject may have progenitor cells that possess acquired mutations within the JAK2, CALR and MPL genes. For example, common mutations in JAK2 include the V617F mutation and mutations (e.g., substitutions, deletions, insertions, duplications) of exon 12. Common mutations in CALR include exon 9 mutations. Common mutations in MPL include exon 10 mutations (e.g., W515L and W515K). Cytogenetic and molecular testing is typically conducted using allele-specific quantitative PCR (qPCR), digital PCR or next-generation sequencing. The foregoing methods are reviewed in Haslam, K. and Langabeer, S. E., “Monitoring Residual Disease in the Myeloproliferative Neoplasms: Current Applications and Emerging Approaches,” Biomed. Res. Intl. 2016:7241591, the relevant teachings of which are incorporated herein by reference in their entireties.

The methods can be defined based on other baseline characteristics. Thus, in some embodiments, the subject has moderate or severe splenomegaly at baseline. In some embodiments, the subject has a baseline spleen size of, e.g., ≥450, 600, 750, 1,000, 1,500, or 2,000 cm³ by imaging. In some embodiments, the subject has a total symptoms score (TSS) at baseline greater than 15, 20, 25, 30, 35, 40, 45, or 50. In some embodiments, the subject is in need of cytokine modulation. In another embodiment the subject has a JAK2V16F mutation. In some embodiments, the subject has a CALR mutation. In some embodiments, the subject has a MPLW515L mutation.

In some embodiments, the subject is characterized by poor bone marrow functioning at baseline. Thus, in some embodiments the subject is characterized by thrombocytopenia at baseline (e.g. a platelet count of ≤150, 100, 75, 50, or 25×10⁹L). In some embodiments the subject is characterized by anemia at baseline (e.g. a Hgb level≤10, 9, or 8 g/dL). In some embodiments, the subject is characterized by transfusion dependence at baseline. In some embodiments, the subject is characterized by serious thrombocytopenia or anemia at baseline (i.e. a platelet count≤50×10⁹/L or a Hgb level≤8 g/dL).

In some embodiments, the subject has grade 2 or higher fibrosis at baseline. In some embodiments, the subject has an absolute neutrophil count of greater than 1×10⁹/L at baseline. In some embodiments, the subject has a peripheral blood blast less than 10% at baseline.

In some embodiments, the myeloproliferative neoplasm is responsive to selective PIM1 inhibition. E.g., the myeloproliferative neoplasm is responsive to a selective PIM1 kinase inhibitor having a PIM1/PIM2 IC₅₀ ratio of less than 0.05, 0.045, 0.04, 0.035, 0.03, or 0.025, wherein IC₅₀ is determined as described in Foulks, Neoplasia Vol. 16, No. 5, 2014.

### Pharmaceutical Compositions

Other embodiments are directed to pharmaceutical compositions. The PIM kinase inhibitor (i.e., Compound (1) or a pharmaceutically acceptable salt thereof) and/or JAK inhibitor (e.g., ruxolitinib, fedratinib, pacritinib, momelotinib, or luspatercept, or a pharmaceutically acceptable salt thereof) and/or ACVR inhibitor (e.g., momelotinib, pacritinib, luspatercept, or a pharmaceutically acceptable salt thereof), ropeginterferon alfa-2b, navtemadelin, or other therapeutic agent may be formulated together or separately according to methods known in the art. Certain embodiments comprise a pharmaceutically acceptable carrier or excipient, a PIM kinase inhibitor and/or a JAK inhibitor and/or ACVR inhibitor. In some embodiments, the pharmaceutical composition comprises a PIM kinase inhibitor and/or JAK inhibitor and/or ACVR inhibitor according to any of the foregoing described embodiments.

In some embodiments, the pharmaceutical composition is formulated for oral administration. In other embodiments, the pharmaceutical composition is formulated for injection.

Suitable routes of administration also include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal routes of administration.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example, subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

In some embodiments, a PIM kinase inhibitor and/or JAK kinase inhibitor and/or ACVR inhibitor is administered in a single dose. Typically, such administration will be by injection, e.g., intravenous injection, in order to introduce the agent quickly. However, other routes are used as appropriate. Accordingly, in some embodiments, a PIM kinase inhibitor (i.e., Compound (1) or a pharmaceutically acceptable salt thereof) and/or JAK kinase inhibitor (e.g., ruxolitinib, fedratinib, pacritinib, momelotinib, or a pharmaceutically acceptable salt thereof) and/or ACVR inhibitor (e.g., momelotinib, pacritinib, luspatercept, or a pharmaceutically acceptable salt thereof) or ropeginterferon alfa-2b or navtemadelin is administered orally.

In some embodiments, a PIM kinase inhibitor (i.e., Compound (1) or a pharmaceutically acceptable salt thereof) a JAK kinase inhibitor, and/or an ACVR administered in multiple doses. In some embodiments, dosing is about once, twice, three times, four times, five times, six times, or more than six times per day. In other embodiments, dosing is about once a month, once every two weeks, once a week, or once every other day. In another embodiment a PIM kinase inhibitor and another agent (e.g., a JAK2 inhibitor) are administered together about once per day to about 6 times per day. In another embodiment the administration of a PIM kinase inhibitor and another agent (e.g., JAK inhibitor) continues for less than about 7 days. In yet another embodiment, the administration continues for more than about 6, 10, 14, 28 days, two months, six months, or one year. In another embodiment, the administration continues for from about seven days to about five years (e.g., from about seven days to about two years, from about seven days to about one year). In another embodiment, the administration continues for 28 days. In another embodiment, the administration continues for one year. In some cases, continuous dosing is achieved and maintained as long as necessary.

Administration of the PIM kinase inhibitor and optionally JAK or ACVR inhibitor may continue as long as necessary. In some embodiments, a PIM kinase inhibitor and optionally JAK or ACVR inhibitor are administered for more than 1, 2, 3, 4, 5, 6, 7, 14, or 28 days. In some embodiments, a PIM kinase inhibitor and optionally JAK or ACVR inhibitor are administered for less than 28, 14, 7, 6, 5, 4, 3, 2, or 1 day. In some embodiments, a PIM kinase inhibitor and optionally JAK or ACVR inhibitor are administered chronically on an ongoing basis, e.g., for the treatment of chronic effects.

In some embodiments, one or more cycles (e.g., 1, 2, 3, 4, 5, 6, 7, 8, 9, 10, 11, 12, etc. cycles) of the PIM kinase inhibitor (i.e., Compound (1) or a pharmaceutically acceptable salt thereof) and/or JAK kinase inhibitor (e.g., ruxolitinib, or a pharmaceutically acceptable salt thereof), and/or an ACVR inhibitor are administered. In some embodiments, the PIM kinase inhibitor (i.e., Compound (1) or a pharmaceutically acceptable salt thereof) and/or JAK kinase inhibitor (e.g., ruxolitinib, or a pharmaceutically acceptable salt thereof), and/or an ACVR inhibitor are administered on a cycle, for example, a 28-day cycle. Accordingly, in some embodiments, one or more cycles of the PIM kinase inhibitor and/or JAK kinase inhibitor (e.g., PIM kinase inhibitor) are each independently administered once or twice per day for 28 days on a 28-day cycle.

In some embodiments, the PIM kinase inhibitor and optionally JAK or ACVR inhibitor are administered in dosages. Due to intrasubject variability in compound pharmacokinetics, individualization of dosing regimen is provided in certain embodiments. Dosing for a compound of embodiments of the disclosure may be found by routine experimentation in light of the instant disclosure and/or can be derived by one of ordinary skill in the art.

In some embodiments, the PIM kinase inhibitors and optionally JAK or ACVR inhibitor are formulated into pharmaceutical compositions. In specific embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

A pharmaceutical composition, as used herein, refers to a mixture of (1) a PIM kinase inhibitor, (2) a combination of a PIM kinase inhibitor and a JAK kinase inhibitor, (3) a combination of a PIM kinase inhibitor and an ACVR inhibitor, or (4) a combination of a PIM kinase inhibitor, and a JAK kinase inhibitor, with other chemical components. Other chemical components may include carriers, stabilizers, diluents, dispersing agents, suspending agents, thickening agents, and/or excipients. In certain embodiments, the pharmaceutical composition facilitates administration of the compound(s) to an organism. In some embodiments, practicing the methods of treatment or use provided herein, therapeutically effective amount(s) of inhibitors of PIM kinase and optionally JAK kinase inhibitors or ACVR inhibitors are administered in a pharmaceutical composition to a human subject having a disease, disorder or medical condition to be treated as provided herein. In certain embodiments, therapeutically effective amounts vary depending on the severity of the disease, the age and relative health of the subject, the potency of the compound used and other factors.

In one embodiment, the inhibitor(s) are formulated in an aqueous solution. In specific embodiments, the aqueous solution is selected from, by way of example only, a physiologically compatible buffer, such as Hank's solution, Ringer's solution, or physiological saline buffer. In other embodiments, inhibitors targeting at least two super-enhancer components are formulated for transmucosal administration. In specific embodiments, transmucosal formulations include penetrants that are appropriate to the barrier to be permeated. In still other embodiments wherein the compounds described herein are formulated for other parenteral injections; appropriate formulations include aqueous or non-aqueous solutions. In specific embodiments, such solutions include physiologically compatible buffers and/or excipients.

In another embodiment, compounds described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropyl methylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as and by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, inhibitors targeting PIM kinase, JAK kinase, and/or ACVR may be formulated into other oral dosage forms. Oral dosage forms include push fit capsules made of gelatin, as well as soft, sealed capsules made of gelatin and a plasticizer, such as glycerol or sorbitol. In specific embodiments, push fit capsules contain the active ingredients in admixture with one or more fillers. Fillers include, by way of example only, lactose, binders such as starches, and/or lubricants such as talc or magnesium stearate and, optionally, stabilizers. In other embodiments, soft capsules contain one or more active compounds that are dissolved or suspended in a suitable liquid. Suitable liquids include, by way of example only, one or more fatty oils, liquid paraffins, or liquid polyethylene glycols. In addition, stabilizers are optionally added.

A particular composition comprises a PIM kinase inhibitor (i.e., Compound (1) or a pharmaceutically acceptable salt thereof), and a polyglycolized glyceride.

“Polyglycolized glycerides” refers to mixtures of monoesters, diesters and triesters of glycerols and monoesters and diesters of polyethylene glycols with a mean relative molecular mass between about 200 and 6000. Polyglycolized glycerides may be obtained by partial transesterification of triglycerides with polyethylene glycol or by esterification of glycerol and polyethylene glycol with fatty acids. In some embodiments, the fatty acid component contains between 8-22 carbon atoms, for example, between 10-18 carbon atoms. Examples of natural vegetable oils from which polyglycolized glycerides can be derived include palm kernel oil and palm oil. Suitable polyol compounds generally have a molecular weight ranging from about 200 to about 6000 g/mol and preferably contain polyethylene glycol, although other polyols may be employed, such as polyglycerols or sorbitol. Polyglycolized glycerides are available on the market under the trade name Gelucire®. Examples of polyglycolized glycerides useful in various embodiments include WL 2514CS, LABRASOL, LABRAFIL, Gelucire 44/14 (lauroyl polyoxy-32 glycerides), Gelucire 33/01, Gelucire 35/10, Gelucire 37/02, Gelucire 50/13, Gelucire 44/11 and mixtures thereof.

“Gelucire® 44/14” or “Gelucire 44/14” is a lipid-based excipient manufactured by Gattefosse Corporation, Westwood, N.J. comprising a mixture of pegylated fatty acid esters and glycerides. The number 44 denotes the melting point of the compound and 14 indicates hydrophile/lipophile balance (HLB) value. Other Gelucire excipients similarly indicate values for melting point and HLB values. For example, Gelucire 33/01, Gelucire 35/10, Gelucire 37/02, Gelucire 50/13 and Gelucire 44/11.

In some embodiments, the composition of the PIM kinase inhibitor and the polyglycolized glyceride is formulated for oral administration, e.g., in the form of tablets, lozenges, aqueous or oily suspensions, dispersible powders or granules, emulsion, hard or soft capsules, or syrups or elixirs.

Compound (1) herein may be in free-base form, or in a pharmaceutically acceptable salt form. In some embodiments, Compound (1) is present as a free base. In some embodiments, Compound (1) is present as a salt. In some embodiments, Compound (1) is present as a hydrochloride salt.

A composition comprising the PIM kinase inhibitor disclosed herein and a polyglycolized glyceride can optionally be used in place of the PIM kinase inhibitor in any of the methods disclosed herein.

In other embodiments, the PIM kinase inhibitor and/or JAK kinase inhibitors and/or ACVR inhibitors are formulated for buccal or sublingual administration. Formulations suitable for buccal or sublingual administration include, by way of example only, tablets, lozenges, or gels. In still other embodiments, the compounds described herein are formulated for parental injection, including formulations suitable for bolus injection or continuous infusion. In specific embodiments, formulations for injection are presented in unit dosage form (e.g., in ampoules) or in multi dose containers. Preservatives are, optionally, added to the injection formulations. In still other embodiments, the pharmaceutical compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water soluble form. In additional embodiments, suspensions of the active compounds (e.g., the PIM kinase inhibitor and/or JAK kinase inhibitors and/or ACVR inhibitors) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

In still other embodiments, the PIM kinase inhibitor and/or JAK kinase inhibitors and/or ACVR inhibitors are administered topically. The compounds described herein are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other embodiments, the PIM kinase inhibitor and/or JAK kinase inhibitors and/or ACVR inhibitors are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of inhibitors is accomplished by means of iontophoretic patches and the like. In certain embodiments, transdermal patches provide controlled delivery of inhibitors. In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In other embodiments, the PIM kinase inhibitors and/or JAK kinase inhibitors and/or ACVR inhibitors are formulated for administration by inhalation. Various forms suitable for administration by inhalation include, but are not limited to, aerosols, mists or powders. Pharmaceutical compositions of inhibitors are conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatin for use in an inhaler or insufflator are formulated containing a powder mix of the compound and a suitable powder base such as lactose or starch.

In still other embodiments, the PIM kinase inhibitors and/or JAK kinase inhibitors and/or ACVR inhibitors are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In certain embodiments, pharmaceutical compositions are formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are optionally used as suitable. Pharmaceutical compositions comprising inhibitors are manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and inhibitors, described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of inhibitors presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the PIM kinase inhibitors and/or JAK kinase inhibitors and/or ACVR inhibitors described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical compositions comprising the PIM kinase inhibitors and/or JAK kinase inhibitors and/or ACVR inhibitors illustratively take the form of a liquid where the agents are present in solution, in suspension or both. Typically, when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, useful aqueous suspensions contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected, for example, from carboxymethylcellulose, carbomer (acrylic acid polymer), poly(methyl methacrylate), polyacrylamide, polycarbophil, acrylic acid/butyl acrylate copolymer, sodium alginate and dextran.

Useful pharmaceutical compositions also, optionally, include solubilizing agents to aid in the solubility of inhibitors. The term “solubilizing agent” generally includes agents that result in formation of a micellar solution or a true solution of the agent. Certain acceptable nonionic surfactants, for example polysorbate 80, are useful as solubilizing agents, as are ophthalmically acceptable glycols, polyglycols, e.g., polyethylene glycol 400, and glycol ethers.

Furthermore, useful pharmaceutical compositions optionally include one or more pH adjusting agents or buffering agents, one or more salts in an amount required to bring osmolality of the composition into an acceptable range, one or more preservatives to inhibit microbial activity, one or more surfactants to enhance physical stability or for other purposes, one or more antioxidants to enhance chemical stability where required, or a combination of two or more thereof.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methyl pyrrolidone are also employed. In additional embodiments, the compounds described herein are delivered using a sustained release system.

In certain embodiments, the formulations described herein comprise one or more antioxidants, metal chelating agents, thiol containing compounds and/or other general stabilizing agents.

The PIM kinase inhibitor and optional JAK inhibitor and/or ACVR inhibitor can be administered concurrently or separately. When multiple therapeutic agents are administered which are not presented in the same dosage form, administration of the various therapeutic agents (e.g. PIM Kinase Inhibitor, and/or JAK inhibitor, and/or ACVR inhibitor) can be administered simultaneously in separate dosage forms, contemporaneously in separate dosage forms, sequentially in separate dosage forms, or per a protocol schedule. For example, one of the inhibitors may be administered via a bolus followed by a separate bolus of the second inhibitor after an appropriate period of time. Slower administration, such as a longer duration infusion can be used for administration of one or both of the inhibitors. The skilled clinician can determine appropriate administration methods and orders, which are all within the scope of the present disclosure.

Some embodiments thus provide a separate dosage form comprising a PIM kinase inhibitor (i.e., Compound (1) or a pharmaceutically acceptable salt thereof) and an additional therapeutic agent (e.g., an additional therapeutic agent described herein), wherein the PIM kinase inhibitor and the additional therapeutic agent are associated with one another. The term “associated with one another,” as used herein, means that the separate dosage forms are packaged together or otherwise attached to one another such that it is readily apparent that the separate dosage forms are intended to be sold and/or administered together, for example, according to a method described herein. In some embodiments, the dosage form comprises a PIM kinase inhibitor (i.e., Compound (1) or a pharmaceutically acceptable salt thereof) and a JAK kinase inhibitor (e.g., ruxolitinib, or a pharmaceutically acceptable salt thereof) and/or an ACVR inhibitor. In some embodiments, the PIM kinase inhibitor is as described herein. In some embodiments, the JAK inhibitor is as described herein. In some embodiments, the ACVR inhibitor is as described herein.

Some related embodiments provide a kit comprising a PIM kinase inhibitor (i.e., Compound (1) or a pharmaceutically acceptable salt thereof) and written instructions for administering the PIM kinase inhibitor for treatment of a myeloproliferative neoplasm is provided. In more specific embodiments, the kit further comprises a JAK inhibitor and written instructions for administering the JAK inhibitor in combination with the PIM kinase inhibitor. In more specific embodiments, the kit further comprises an ACVR inhibitor and written instructions for administering the JAK inhibitor in combination with the PIM kinase inhibitor. In some embodiments, the PIM kinase inhibitor is as described herein. In some embodiments, the JAK inhibitor is as described herein. In some embodiments, the ACVR inhibitor is as described herein. In some embodiments, the myeloproliferative neoplasm is as described herein.

The PIM kinase inhibitors, such as Compound (1) or a pharmaceutically acceptable salt thereof and optional JAK inhibitors and/or optional ACVR can be prepared according to methods known in the art. Exemplary preparation procedures are provided in PCT Pub. Nos: WO 2016/161248; WO 2014/052365; WO 2015/048689; WO 2015/002894; WO 2014/168975; WO 2014/159745; WO 2014/130693; WO 2014/078578; WO 2014/018567; WO 2013/184572; WO 2013/173518; WO 2013/116382; WO 2013/102059; WO 2013/059738; WO 2013/010136; WO 2013/013188; WO 2011/153514; WO 2011/046964; WO 2010/009342; WO 2008/121742; WO 2008/054827; WO 2008/039218; WO 2008/058126; WO 2007/087068; and in U.S. Pub. Nos: 2015/0018336; 2014/0336206; 2014/0329807; 2014/0243355; 2014/0212485; 2014/0194446/2014/0187564; 2014/0135347; 2014/0128414; 2014/0187565; 2014/0171453; 2014/0163027; 2014/01663046; 2014/0142126; 2014/0142123; 2014/0128413; 2014/0079690; 2014/0080844; 2014/0057907; 2014/0039168; 2013/0338172; 2013/0310402; 2013/0273030; 2013/0197014; 2013/0035334; 2013/0012525; 2012/0283277; 2012/0283276; 2012/0277254; 2012/0252821; 2010/0331350, and U.S. Pat. No. 7,750,007, the full disclosures of which are hereby incorporated by reference.

### Example Embodiments of the Technology

Embodiment 1. A method for treating a myeloproliferative neoplasm in a human subject in need thereof, comprising administering to the subject a therapeutically effective amount of Compound (1) or a pharmaceutically acceptable salt thereof, wherein the subject has poor bone marrow functioning characterized by: (a) thrombocytopenia (e.g. a platelet count of ≤150, 100, 75, 50, or 25×10⁹/L); (b) anemia (e.g. a Hgb level≤10, 9, or 8 g/dL); (c) transfusion dependence; or (d) a combination thereof, and wherein the treatment induces a clinically meaningful response in the myeloproliferative neoplasm.

Embodiment 2. A method for treating a myeloproliferative neoplasm in a human subject in need thereof, comprising administering to the subject a therapeutically effective amount of a selective PIM1 inhibitor, wherein the subject has poor bone marrow functioning characterized by: (a) thrombocytopenia (e.g. a platelet count of ≤150, 100, 75, 50, or 25×10⁹/L); (b) anemia (e.g. a Hgb level≤10, 9, or 8 g/dL); (c) transfusion dependence; or (d) a combination thereof, wherein the treatment induces a clinically meaningful response in the myeloproliferative neoplasm.

Embodiment 3. A method of treating a myeloproliferative neoplasm in a human subject in need thereof, wherein the subject has previously been treated by a JAK inhibitor for the myeloproliferative neoplasm and optionally has relapsed disease or is refractory to the JAK inhibitor, comprising administering to the subject a therapeutically effective amount of Compound (1) or a pharmaceutically acceptable salt thereof, wherein the treatment induces a clinically meaningful response in the myeloproliferative neoplasm.

Embodiment 4. A method of treating a myeloproliferative neoplasm by inducing TSS50 in a human subject in need thereof, comprising administering to the subject a therapeutically effective amount of Compound (1) or a pharmaceutically acceptable salt thereof, wherein the myeloproliferative neoplasm is treated by inducing TSS50 in the subject, optionally at a time point between and including 24 and 48 weeks.

Embodiment 5. A method of treating a myeloproliferative neoplasm in a human subject in need thereof, comprising administering to the subject Compound (1) or a pharmaceutically acceptable salt thereof, and ruxolitinib or momelotinib or a pharmaceutically acceptable salt thereof in amounts that induce no overlapping hematological toxicities (e.g., anemia and/or thrombocytopenia and/or transfusion dependence), wherein the treatment induces a clinically meaningful response in the myeloproliferative neoplasm.

Embodiment 6. A method of treating a myeloproliferative neoplasm in a human subject in need thereof, comprising administering to the subject a selective PIM1 inhibitor and ruxolitinib or momelotinib or a pharmaceutically acceptable salt thereof in amounts that induce no overlapping hematological toxicities (e.g., anemia and/or thrombocytopenia and/or transfusion dependence), wherein the treatment induces a clinically meaningful response in the myeloproliferative neoplasm.

Embodiment 7. A method of inducing a probability of SVR35 in a human subject affected by a myeloproliferative neoplasm≥15% or 20%, wherein the subject has previously been treated by a JAK inhibitor for the myeloproliferative neoplasm and optionally has relapsed disease or is refractory to the JAK inhibitor, comprising administering to the subject a therapeutically effective amount of Compound (1) or a pharmaceutically acceptable salt thereof, wherein the treatment induces a probability of SVR35 in the human subject≥15% or 20%.

Embodiment 8. A method of inducing a probability of TSS50 in a human subject affected by a myeloproliferative neoplasm≥30%, 35%, 40%, 45%, or 50%, wherein the subject has previously been treated by a JAK inhibitor for the myeloproliferative neoplasm and optionally has relapsed disease or is refractory to the JAK inhibitor, comprising administering to the subject a therapeutically effective amount of Compound (1), or a pharmaceutically acceptable salt thereof, wherein the treatment induces a probability of TSS50 in the human subject≥30%, 35%, 40%, 45%, or 50%.

Embodiment 9: A method for treating a myeloproliferative neoplasm in a human subject in need thereof, comprising administering to the subject a therapeutically effective amount of a selective PIM1 inhibitor, wherein the subject has a baseline platelet count of less than or equal to 200×10⁹/L (e.g., less than or equal to 150, 100, 75, 50, or 25×10⁹L).

Embodiment 10: A method for treating a myeloproliferative neoplasm in a human subject in need thereof, comprising administering to the subject a therapeutically effective amount of Compound (1) or a pharmaceutically acceptable salt thereof, wherein the subject has a baseline platelet count of less than or equal to 200×10⁹/L (e.g., less than or equal to 150, 100, 75, 50, or 25×10⁹L).

Embodiment 11: A method for treating a myeloproliferative neoplasm in a human subject in need thereof, comprising:
administering to the subject, a therapeutically effective amount of a selective PIM1 inhibitor, and
continuing administering to the subject a therapeutically effective amount of the selective PIM1 inhibitor,
wherein:
the subject has a platelet count reduction of (a) less than 1 grade, or (b) less than or equal to 25×10⁹/L, and/or (c) less than or equal to 35%, and
the platelet count reduction is determined by a change in the baseline platelet count determined within one week prior to administering the selective PIM1 inhibitor and a second platelet count determined at least 2 weeks after administering.

Embodiment 12: A method for treating a myeloproliferative neoplasm in a human subject in need thereof, comprising administering to the subject, a therapeutically effective amount of Compound (1) or a pharmaceutically acceptable salt thereof; and
continuing administering to the subject a therapeutically effective amount of Compound (1) or the pharmaceutically acceptable salt,
wherein:
the subject has a platelet count reduction of (a) less than 1 grade, or (b) less than or equal to 25×10⁹/L, and/or (c) less than or equal to 35%, and
the platelet count reduction is determined by a change in the baseline platelet count determined within one week prior to administering Compound (1) or the pharmaceutically acceptable salt and a second platelet count determined at least 2 weeks after administering.

Embodiment 13: A method for treating a myeloproliferative neoplasm in a human subject in need thereof, comprising administering to the subject a selective PIM1 inhibitor,
wherein:
   the subject has had one or more previous treatments comprising the selective PIM1 inhibitor and has a platelet count reduction of (a) less than 1 grade, (b) less than or equal to 25×10⁹/L, and/or (c) less than or equal to 35%,
   the platelet count reduction is determined by a change in the baseline platelet count determined within one week prior to administering the selective PIM1 inhibitor and a second platelet count determined at least 2 weeks after administering, and
   the method results in the subject exhibiting bone marrow fibrosis reduction, total symptom reduction, cytokine modulation, or a combination of two or more thereof.

Embodiment 14: A method for treating a myeloproliferative neoplasm in a human subject in need thereof, comprising administering to the subject a therapeutically effective amount of Compound (1) Compound (1) or a pharmaceutically acceptable salt thereof, wherein:
the subject has had one or more previous treatments comprising Compound (1) or the pharmaceutically acceptable salt and has a platelet count reduction of (a) less than 1 grade, (b) less than or equal to 25×10⁹/L, and/or (c) less than or equal to 35%,
the platelet count reduction is determined by a change in the baseline platelet count determined within one week prior to administering Compound (1) or the pharmaceutically acceptable salt and a second platelet count determined at least 2 weeks after administering, and
the method results in the subject exhibiting bone marrow fibrosis reduction, total symptom reduction, cytokine modulation, or a combination of two or more thereof.

Embodiment 15: A method for treating a myeloproliferative neoplasm in a human subject in need thereof, comprising administering to the subject a therapeutically effective amount of a selective PIM1 inhibitor and ruxolitinib, fedratinib, pacritinib, momelotinib, luspatercept, ropeginterferon alfa-2b, navtemadelin, or a pharmaceutically acceptable salt thereof, or a combination of two or more thereof,
wherein:
   the subject has a baseline platelet count of less than or equal to 200×10⁹/L (e.g., less than or equal to 150, 100, 75, 50, or 25×10⁹/);
   ruxolitinib or the pharmaceutically acceptable salt is in a dose of about 5 mg to about 25 mg;
   fedratinib or the pharmaceutically acceptable salt is in a dose of about 200 mg to about 500 mg;
   pacritinib or the pharmaceutically acceptable salt is in a dose of about 100 mg to about 200 mg;
   momelotinib or the pharmaceutically acceptable salt is in a dose of about 50 mg to about 200 mg;
   luspatercept is in a dose of about 0.5 mg/kg to 1.75 mg/kg based on the subject's body weight;
   ropeginterferon alfa-2b or the pharmaceutically acceptable salt is in a dose of 50 to 500 g; and
   navtemadelin or the pharmaceutically acceptable salt is in a dose of 120 to 240 mg.

Embodiment 16: A method for treating a myeloproliferative neoplasm in a human subject in need thereof, comprising administering to the subject a therapeutically effective amount of Compound (1) or a pharmaceutically acceptable salt thereof; and ruxolitinib, fedratinib, pacritinib, momelotinib, luspatercept, ropeginterferon alfa-2b, navtemadelin, or a pharmaceutically acceptable salt thereof, or a combination of two or more thereof;
wherein:
   the subject has a baseline platelet count of less than or equal to 200×10⁹/L (e.g., less than or equal to 150, 100, 75, 50, or 25×10⁹/);
   ruxolitinib or the pharmaceutically acceptable salt is in a dose of about 5 mg to about 25 mg;
   fedratinib or the pharmaceutically acceptable salt is in a dose of about 200 mg to about 500 mg;
   pacritinib or the pharmaceutically acceptable salt is in a dose of about 100 mg to about 200 mg;
   momelotinib or the pharmaceutically acceptable salt is in a dose of about 50 mg to about 200 mg;
   luspatercept is in a dose of about 0.5 mg/kg to 1.75 mg/kg based on the subject's body weight;
   ropeginterferon alfa-2b or the pharmaceutically acceptable salt is in a dose of 50 to 500 g; and
   navtemadelin or the pharmaceutically acceptable salt is in a dose of 120 to 240 mg.

Embodiment 17: A method for treating a myeloproliferative neoplasm in a human subject in need thereof, comprising administering to the subject a therapeutically effective amount of Compound (1) or a pharmaceutically acceptable salt thereof;
wherein:
   the subject has a baseline platelet count of less than or equal to 200×10⁹/L (e.g., less than or equal to 150, 100, 75, 50, or 25×10⁹L).

Embodiment 18: A method for treating a myeloproliferative neoplasm in a human subject in need thereof, comprising administering to the subject a therapeutically effective amount of a selective PIM1 inhibitor and ruxolitinib or a pharmaceutically acceptable salt thereof;
wherein:
   the subject has a baseline platelet count of less than or equal to 200×10⁹/L (e.g., less than or equal to 150, 100, 75, 50, or 25×10⁹/L); and
   ruxolitinib or the pharmaceutically acceptable salt is in a dose of about 5 mg to about 25 mg.

Embodiment 19: A method for treating a myeloproliferative neoplasm in a human subject in need thereof, comprising administering to the subject a therapeutically effective amount of Compound (1), or a pharmaceutically acceptable salt thereof; and ruxolitinib or a pharmaceutically acceptable salt thereof;
wherein:
   the subject has a baseline platelet count of less than or equal to 200×10⁹/L (e.g., less than or equal to 150, 100, 75, 50, or 25×10⁹/L); and
   ruxolitinib or the pharmaceutically acceptable salt is in a dose of about 5 mg to about 25 mg.

Embodiment 20: A method for treating a myeloproliferative neoplasm in a human subject in need thereof, comprising administering to the subject a therapeutically effective amount of a selective PIM1 inhibitor and fedratinib or a pharmaceutically acceptable salt thereof;
wherein:
   the subject has a baseline platelet count of less than or equal to 200×10⁹/L (e.g., less than or equal to 150, 100, 75, 50, or 25×10⁹/L); and
   fedratinib or the pharmaceutically acceptable salt is in a dose of about 200 mg to about 500 mg.

Embodiment 21: A method for treating a myeloproliferative neoplasm in a human subject in need thereof, comprising administering to the subject a therapeutically effective amount of Compound (1) or a pharmaceutically acceptable salt thereof; and fedratinib or a pharmaceutically acceptable salt thereof;
wherein:
   the subject has a baseline platelet count of less than or equal to 200×10⁹/L (e.g., less than or equal to 150, 100, 75, 50, or 25×10⁹/L); and
   fedratinib or the pharmaceutically acceptable salt is in a dose of about 200 mg to about 500 mg.

Embodiment 22: A method for treating a myeloproliferative neoplasm in a human subject in need thereof, comprising administering to the subject a therapeutically effective amount of a selective PIM1 inhibitor and pacritinib or a pharmaceutically acceptable salt thereof;
wherein:
   the subject has a baseline platelet count of less than or equal to 200×10⁹/L (e.g., less than or equal to 150, 100, 75, 50, or 25×10⁹/L); and
   pacritinib or the pharmaceutically acceptable salt is in a dose of about 100 mg to about 200 mg.

Embodiment 23: A method for treating a myeloproliferative neoplasm in a human subject in need thereof, comprising administering to the subject a therapeutically effective amount of Compound (1) or a pharmaceutically acceptable salt thereof; and pacritinib or a pharmaceutically acceptable salt thereof;
wherein:
   the subject has a baseline platelet count of less than or equal to 200×10⁹/L (e.g., less than or equal to 150, 100, 75, 50, or 25×10⁹/L); and
   pacritinib or the pharmaceutically acceptable salt is in a dose of about 100 mg to about 200 mg.

Embodiment 24: A method for treating a myeloproliferative neoplasm in a human subject in need thereof, comprising administering to the subject a therapeutically effective amount of a selective PIM1 inhibitor and momelotinib or a pharmaceutically acceptable salt thereof;
wherein:
   the subject has a baseline platelet count of less than or equal to 200×10⁹/L (e.g., less than or equal to 150, 100, 75, 50, or 25×10⁹/L); and
   momelotinib or the pharmaceutically acceptable salt is in a dose of about 50 mg to about 200 mg.

Embodiment 25: A method for treating a myeloproliferative neoplasm in a human subject in need thereof, comprising administering to the subject a therapeutically effective amount of Compound (1) or a pharmaceutically acceptable salt thereof; and momelotinib or a pharmaceutically acceptable salt thereof;
wherein:
   the subject has a baseline platelet count of less than or equal to 200×10⁹/L (e.g., less than or equal to 150, 100, 75, 50, or 25×10⁹/L); and
   momelotinib or the pharmaceutically acceptable salt is in a dose of about 50 mg to about 200 mg.

Embodiment 26: A method for treating a myeloproliferative neoplasm in a human subject in need thereof, comprising administering to the subject a therapeutically effective amount of a selective PIM1 inhibitor and luspatercept;
wherein:
   the subject has a baseline platelet count of less than or equal to 200×10⁹/L (e.g., less than or equal to 150, 100, 75, 50, or 25×10⁹/L); and
   luspatercept is in a dose of about 0.5 mg/kg or 1.75 mg/kg based on the subject's body weight.

Embodiment 27: A method for treating a myeloproliferative neoplasm in a human subject in need thereof, comprising administering to the subject a therapeutically effective amount of Compound (1) or a pharmaceutically acceptable salt thereof; and luspatercept;
wherein:
   the subject has a baseline platelet count of less than or equal to 200×10⁹/L (e.g., less than or equal to 150, 100, 75, 50, or 25×10⁹/L); and
   luspatercept is in a dose of about 0.5 mg/kg or 1.75 mg/kg based on the subject's body weight.

Embodiment 28: A method for treating a myeloproliferative neoplasm in a human subject in need thereof, comprising administering to the subject a therapeutically effective amount of a selective PIM1 inhibitor and ruxolitinib or a pharmaceutically acceptable salt thereof;
wherein:
   the subject has a baseline platelet count of greater than or equal to 10×10⁹/L (e.g., great than or equal to 15, 25, 50, 75, or 100×10⁹/L); and
   ruxolitinib or the pharmaceutically acceptable salt is in a dose of about 5 mg to about 25 mg.

Embodiment 29: A method for treating a myeloproliferative neoplasm in a human subject in need thereof, comprising administering to the subject a therapeutically effective amount of Compound (1) or a pharmaceutically acceptable salt thereof; and ruxolitinib or a pharmaceutically acceptable salt thereof;
wherein:
   the subject has a baseline platelet count of greater than or equal to 10×10⁹/L (e.g., great than or equal to 15, 25, 50, 75, or 100×10⁹/L); and
   ruxolitinib or the pharmaceutically acceptable salt is in a dose of about 5 mg to about 25 mg.

Embodiment 30: A method for treating a myeloproliferative neoplasm in a human subject in need thereof, comprising administering to the subject a therapeutically effective amount of a selective PIM1 inhibitor and fedratinib or a pharmaceutically acceptable salt thereof;
wherein:
   the subject has a baseline platelet count of greater than or equal to 10×10⁹/L (e.g., great than or equal to 15, 25, 50, 75, or 100×10⁹/L); and
   fedratinib or the pharmaceutically acceptable salt is in a dose of about 200 mg to about 500 mg.

Embodiment 31: A method for treating a myeloproliferative neoplasm in a human subject in need thereof, comprising administering to the subject a therapeutically effective amount of Compound (1) or a pharmaceutically acceptable salt thereof; and fedratinib or a pharmaceutically acceptable salt thereof;
wherein:
   the subject has a baseline platelet count of greater than or equal to 10×10⁹/L (e.g., great than or equal to 15, 25, 50, 75, or 100×10⁹/L); and
   fedratinib or the pharmaceutically acceptable salt is in a dose of about 200 mg to about 500 mg.

Embodiment 32: A method for treating a myeloproliferative neoplasm in a human subject in need thereof, comprising administering to the subject a therapeutically effective amount of a selective PIM1 inhibitor and pacritinib or a pharmaceutically acceptable salt thereof;
wherein:
   the subject has a baseline platelet count of greater than or equal to 10×10⁹/L (e.g., great than or equal to 15, 25, 50, 75, or 100×10⁹/L); and
   pacritinib or the pharmaceutically acceptable salt is in a dose of about 100 mg to about 200 mg.

Embodiment 33: A method for treating a myeloproliferative neoplasm in a human subject in need thereof, comprising administering to the subject a therapeutically effective amount of Compound (1) or a pharmaceutically acceptable salt thereof; and pacritinib or a pharmaceutically acceptable salt thereof;
wherein:
   the subject has a baseline platelet count of greater than or equal to 10×10⁹/L (e.g., great than or equal to 15, 25, 50, 75, or 100×10⁹/L); and
   pacritinib or the pharmaceutically acceptable salt is in a dose of about 100 mg to about 200 mg.

Embodiment 34: A method for treating a myeloproliferative neoplasm in a human subject in need thereof, comprising administering to the subject a therapeutically effective amount of a selective PIM1 inhibitor and momelotinib or a pharmaceutically acceptable salt thereof;
wherein:
   the subject has a baseline platelet count of greater than or equal to 10×10⁹/L (e.g., great than or equal to 15, 25, 50, 75, or 100×10⁹/L); and
   momelotinib or the pharmaceutically acceptable salt is in a dose of about 50 mg to about 200 mg.

Embodiment 35: A method for treating a myeloproliferative neoplasm in a human subject in need thereof, comprising administering to the subject a therapeutically effective amount of Compound (1) or a pharmaceutically acceptable salt thereof; and momelotinib or a pharmaceutically acceptable salt thereof;
wherein:
   the subject has a baseline platelet count of greater than or equal to 10×10⁹/L (e.g., great than or equal to 15, 25, 50, 75, or 100×10⁹/L); and
   momelotinib or the pharmaceutically acceptable salt is in a dose of about 50 mg to about 200 mg.

Embodiment 36: A method for treating a myeloproliferative neoplasm in a human subject in need thereof, comprising administering to the subject a therapeutically effective amount of a selective PIM1 inhibitor and luspatercept;
wherein:
   the subject has a baseline platelet count of greater than or equal to 10×10⁹/L (e.g., great than or equal to 15, 25, 50, 75, or 100×10⁹/L); and
   luspatercept is in a dose of about 0.5 mg/kg or 1.75 mg/kg based on the subject's body weight.

Embodiment 37: A method for treating a myeloproliferative neoplasm in a human subject in need thereof, comprising administering to the subject a therapeutically effective amount of Compound (1) or a pharmaceutically acceptable salt thereof; and luspatercept;
wherein:
   the subject has a baseline platelet count of greater than or equal to 10×10⁹/L (e.g., great than or equal to 15, 25, 50, 75, or 100×10⁹/L); and
   luspatercept is in a dose of about 0.5 mg/kg or 1.75 mg/kg based on the subject's body weight.

Embodiment 38: A method of treating a myeloproliferative neoplasm in a human subject in need thereof, without causing a clinically significant risk of adverse events, comprising:
administering to the subject a dose from about 5 mg to about 25 mg of ruxolitinib or a pharmaceutically acceptable salt thereof for a period of up to about 36 months;
determining or having determined if the subject had an adverse event during the treatment period;
reducing the dose amount from about 33% to about 80%, or temporarily stopping the dose; and
administering to the subject a therapeutically effective amount of a selective PIM1 inhibitor.

Embodiment 39: A method of treating a myeloproliferative neoplasm in a human subject in need thereof, without causing a clinically significant risk of adverse events, comprising:
administering to the subject a dose from about 5 mg to about 25 mg of ruxolitinib or a pharmaceutically acceptable salt thereof for a period of up to about 36 months;
determining or having determined if the subject had an adverse event during the treatment period;
reducing the dose amount from about 33% to about 80%, or temporarily stopping the dose; and
administering to the subject a therapeutically effective amount of Compound (1) or a pharmaceutically acceptable salt thereof.

Embodiment 40: The method of embodiment 38 or 39, wherein the adverse event is thrombocytopenia, anemia, bruising, dizziness, headache, diarrhea, or a combination of two or more thereof.

Embodiment 41: A method of treating a myeloproliferative neoplasm in a human subject in need thereof while the subject is undergoing a ruxolitinib therapy comprising about 5 mg to about 25 mg of ruxolitinib or a pharmaceutically acceptable salt thereof, the method comprises:
administering to the subject a therapeutically effective amount of a selective PIM1 inhibitor; and
continuing to administer to the subject the therapeutically effective amount of the selective PIM1 inhibitor, during which the subject either (a) does not receive the ruxolitinib therapy, or (b) receives a reduced amount of the ruxolitinib therapy.

Embodiment 42: A method of treating a myeloproliferative neoplasm in a human subject in need thereof while the subject is undergoing a ruxolitinib therapy comprising about 5 mg to about 25 mg of ruxolitinib or a pharmaceutically acceptable salt thereof, the method comprises:
administering to the subject a therapeutically effective amount of Compound (1), or a pharmaceutically acceptable salt thereof, and
continuing to administer to the subject the therapeutically effective amount of Compound (1) or the pharmaceutically acceptable salt, during which the subject either (a) does not receive the ruxolitinib therapy, or (b) receives a reduced amount of the ruxolitinib therapy.

Embodiment 43: The method of embodiment 41 or 42, wherein the reduced amount of the ruxolitinib therapy comprises ≤50% or 75% of the ruxolitinib therapy.

Embodiment 36: A method of treating a myeloproliferative neoplasm in a human subject in need thereof, without causing a clinically significant risk of adverse events, comprising:
administering to the subject a dose from about 200 mg to about 500 mg of fedratinib or a pharmaceutically acceptable salt thereof for a period of up to about 36 months;
determining or having determined if the subject had an adverse event during the treatment period;
reducing the dose amount from about 33% to about 80%, or temporarily stopping the dose; and
administering to the subject a therapeutically effective amount of a selective PIM1 inhibitor.

Embodiment 45: A method of treating a myeloproliferative neoplasm in a human subject in need thereof, without causing a clinically significant risk of adverse events, comprising:
administering to the subject a dose from about 200 mg to about 500 mg of fedratinib or a pharmaceutically acceptable salt thereof for a period of up to about 36 months;
determining or having determined if the subject had an adverse event during the treatment period;
reducing the dose amount from about 33% to about 80%, or temporarily stopping the dose; and
administering to the subject a therapeutically effective amount of Compound (1) or a pharmaceutically acceptable salt thereof.

Embodiment 46: The method of embodiment 44 or 45, wherein the adverse event is thrombocytopenia, anemia diarrhea, nausea, vomiting, encephalopathy, or a combination of two or more thereof.

Embodiment 47: A method of treating a myeloproliferative neoplasm in a human subject in need thereof while the subject is undergoing a fedratinib therapy comprising from about 200 mg to about 500 mg of fedratinib or a pharmaceutically acceptable salt thereof, the method comprises:
administering to the subject a therapeutically effective amount of a selective PIM1 inhibitor; and
continuing to administer to the subject the therapeutically effective amount of the selective PIM1 inhibitor, during which the subject either (a) does not receive the fedratinib therapy, or (b) receives a reduced amount of the fedratinib therapy.

Embodiment 48: A method of treating a myeloproliferative neoplasm in a human subject in need thereof while the subject is undergoing a fedratinib therapy comprising from about 200 mg to about 500 mg of fedratinib or a pharmaceutically acceptable salt thereof, the method comprises:
administering to the subject a therapeutically effective amount of Compound (1), or a pharmaceutically acceptable salt thereof, and
continuing to administer to the subject the therapeutically effective amount of Compound (1) or the pharmaceutically acceptable salt, during which the subject either (a) does not receive the fedratinib therapy, or (b) receives a reduced amount of the fedratinib therapy.

Embodiment 49: The method of embodiment 47 or 48, wherein the reduced amount of the fedratinib therapy comprises ≤50% or 75% of the fedratinib therapy.

Embodiment 50: A method of treating a myeloproliferative neoplasm in a human subject in need thereof, without causing a clinically significant risk of adverse events, comprising:
administering to the subject a dose from about 100 mg to about 200 mg of pacritinib or a pharmaceutically acceptable salt thereof for a period of up to about 36 months;
determining or having determined if the subject had an adverse event during the treatment period;
reducing the dose amount from about 33% to about 80%, or temporarily stopping the dose; and
administering to the subject a therapeutically effective amount of a selective PIM1 inhibitor.

Embodiment 51: A method of treating a myeloproliferative neoplasm in a human subject in need thereof, without causing a clinically significant risk of adverse events, comprising:
administering to the subject a dose from about 100 mg to about 200 mg of pacritinib or a pharmaceutically acceptable salt thereof for a period of up to about 36 months;
determining or having determined if the subject had an adverse event during the treatment period;
reducing the dose amount from about 33% to about 80%, or temporarily stopping the dose; and
administering to the subject a therapeutically effective amount of Compound (1) or a pharmaceutically acceptable salt thereof.

Embodiment 52: The method of embodiment 50 or 51, wherein the adverse event is thrombocytopenia, anemia diarrhea, nausea, peripheral edema, or a combination of two or more thereof.

Embodiment 53: A method of treating a myeloproliferative neoplasm in a human subject in need thereof while the subject is undergoing a pacritinib therapy comprising about 100 mg to about 200 mg of pacritinib or a pharmaceutically acceptable salt thereof, the method comprises:
administering to the subject a therapeutically effective amount of a selective PIM1 inhibitor; and
continuing to administer to the subject the therapeutically effective amount of the selective PIM1 inhibitor, during which the subject either (a) does not receive the pacritinib therapy, or (b) receives a reduced amount of the pacritinib therapy.

Embodiment 54: A method of treating a myeloproliferative neoplasm in a human subject in need thereof while the subject is undergoing a pacritinib therapy comprising about 100 mg to about 200 mg of pacritinib or a pharmaceutically acceptable salt thereof, the method comprises:
administering to the subject a therapeutically effective amount of Compound (1) or a pharmaceutically acceptable salt thereof; and
continuing to administer to the subject the therapeutically effective amount of Compound (1) or the pharmaceutically acceptable salt, during which the subject either (a) does not receive the pacritinib therapy, or (b) receives a reduced amount of the pacritinib therapy.

Embodiment 55: The method of embodiment 53 or 54, wherein the reduced amount of the pacritinib therapy comprises ≤50% or 75% of the pacritinib therapy.

Embodiment 56: A method of treating a myeloproliferative neoplasm in a human subject in need thereof, without causing a clinically significant risk of adverse events, comprising:
administering to the subject a dose from about 50 mg to about 200 mg of momelotinib or a pharmaceutically acceptable salt thereof for a period of up to about 36 months;
determining or having determined if the subject had an adverse event during the treatment period;
reducing the dose amount from about 33% to about 80%, or temporarily stopping the dose; and
administering to the subject a therapeutically effective amount of a selective PIM1 inhibitor.

Embodiment 57: A method of treating a myeloproliferative neoplasm in a human subject in need thereof, without causing a clinically significant risk of adverse events, comprising:
administering to the subject a dose from about 50 mg to about 200 mg of momelotinib or a pharmaceutically acceptable salt thereof for a period of up to about 36 months;
determining or having determined if the subject had an adverse event during the treatment period;
reducing the dose amount from about 33% to about 80%, or temporarily stopping the dose; and
administering to the subject a therapeutically effective amount of Compound (1), or a pharmaceutically acceptable salt thereof.

Embodiment 58: The method of embodiment 56 or 57, wherein the adverse event is thrombocytopenia, anemia diarrhea, nausea, dizziness, or a combination of two or more thereof.

Embodiment 59: A method of treating a myeloproliferative neoplasm in a human subject in need thereof while the subject is undergoing a momelotinib therapy comprising from about 50 mg to about 200 mg of momelotinib or a pharmaceutically acceptable salt thereof, wherein the method comprises:
administering to the subject a therapeutically effective amount of a selective PIM1 inhibitor; and
continuing to administer to the subject the therapeutically effective amount of the selective PIM1 inhibitor, during which the subject either (a) does not receive the momelotinib therapy, or (b) receives a reduced amount of the momelotinib therapy.

Embodiment 60: A method of treating a myeloproliferative neoplasm in a human subject in need thereof while the subject is undergoing a momelotinib therapy comprising from about 50 mg to about 200 mg of momelotinib or a pharmaceutically acceptable salt thereof, wherein the method comprises:
administering to the subject a therapeutically effective amount of Compound (1), or a pharmaceutically acceptable salt thereof, and
continuing to administer to the subject the therapeutically effective amount of Compound (1) or the pharmaceutically acceptable salt, during which the subject either (a) does not receive the momelotinib therapy, or (b) receives a reduced amount of the momelotinib therapy.

Embodiment 61: The method of embodiment 59 or 60, wherein the reduced amount of the momelotinib therapy comprises ≤50% or 75% of the momelotinib therapy.

Embodiment 62: A method of treating a myeloproliferative neoplasm in a human subject in need thereof, without causing a clinically significant risk of adverse events, comprising:
administering to the subject a dose from about 0.6 mg/kg or 1.75 mg/kg of luspatercept for a period of up to about 36 months;
determining or having determined if the subject had an adverse event during the treatment period;
reducing the dose amount from about 33% to about 80%, or temporarily stopping the dose; and
administering to the subject a therapeutically effective amount of a selective PIM1 inhibitor.

Embodiment 63: A method of treating a myeloproliferative neoplasm in a human subject in need thereof, without causing a clinically significant risk of adverse events, comprising:
administering to the subject a dose from about 0.6 mg/kg or 1.75 mg/kg of luspatercept for a period of up to about 36 months;
determining or having determined if the subject had an adverse event during the treatment period;
reducing the dose amount from about 33% to about 80%, or temporarily stopping the dose; and
administering to the subject a therapeutically effective amount of Compound (1) or a pharmaceutically acceptable salt thereof.

Embodiment 64: The method of embodiment 62 or 63, wherein the adverse event is fatigue, headache, musculoskeletal pain, arthralgia, dizziness/vertigo, nausea, diarrhea, cough, abdominal pain, dyspnea, hypersensitivity, or a combination of two or more thereof.

Embodiment 65: A method of treating a myeloproliferative neoplasm in a human subject in need thereof while the subject is undergoing a luspatercept therapy comprising from about 0.5 mg/kg or 1.75 mg/kg of luspatercept, wherein the method comprises:
administering to the subject a therapeutically effective amount of a selective PIM1 inhibitor; and
continuing to administer to the subject the therapeutically effective amount of the selective PIM1 inhibitor, during which the subject either (a) does not receive the luspatercept therapy, or (b) receives a reduced amount of the luspatercept therapy.

Embodiment 66: A method of treating a myeloproliferative neoplasm in a human subject in need thereof while the subject is undergoing a luspatercept therapy comprising from about 0.5 mg/kg or 1.75 mg/kg of luspatercept, wherein the method comprises:
administering to the subject a therapeutically effective amount of Compound (1) or a pharmaceutically acceptable salt thereof; and
continuing to administer to the subject the therapeutically effective amount of Compound (1) or the pharmaceutically acceptable salt, during which the subject either (a) does not receive the luspatercept therapy, or (b) receives a reduced amount of the luspatercept therapy.

Embodiment 67: The method of embodiment 65 or 66, wherein the reduced amount of the luspatercept therapy comprises ≤50% or 75% of the luspatercept therapy.

Embodiment 67a: A method of treating a myeloproliferative neoplasm in a human subject in need thereof, without causing a clinically significant risk of adverse events, comprising administering to the subject therapeutically effective doses of ropeginterferon alfa-2b and Compound (1) or pharmaceutically acceptable salts thereof.

Embodiment 67b: A method of treating a myeloproliferative neoplasm in a human subject in need thereof, without causing a clinically significant risk of adverse events, comprising administering to the subject therapeutically effective doses of navtemadelin and Compound (1) or pharmaceutically acceptable salts thereof.

Embodiment 68. The method of any of the foregoing embodiments wherein the subject has poor bone marrow functioning characterized by thrombocytopenia (e.g. a platelet count of ≤150, 100, 75, 50, or 25×10⁹/).

Embodiment 69. The method of any of the foregoing Embodiments wherein the subject has poor bone marrow functioning characterized by anemia (e.g. a Hgb level≤10, 9, or 8 g/dL).

Embodiment 70. The method of any of the foregoing Embodiments wherein the subject has poor bone marrow functioning characterized by transfusion dependence.

Embodiment 71. The method of any of the foregoing Embodiments wherein the subject has poor bone marrow functioning characterized by a platelet count of ≤100×10⁹/L.

Embodiment 72. The method of any of the foregoing Embodiments wherein the subject has poor bone marrow functioning characterized by a platelet count of ≤50×10⁹/L.

Embodiment 73. The method of any of the foregoing Embodiments wherein the subject has poor bone marrow functioning characterized by a Hgb level≤10 g/dL.

Embodiment 74. The method of any of the foregoing Embodiments wherein the subject has poor bone marrow functioning characterized by a Hgb level≤8 g/dL.

Embodiment 75. The method of any of the foregoing Embodiments wherein the subject has poor bone marrow functioning characterized by transfusion dependence.

Embodiment 76. The method of any of the foregoing Embodiments, wherein the subject has poor bone marrow functioning characterized by: (a) a platelet count of ≤50×10⁹/L; (b) a Hgb level≤8 g/dL; (c) transfusion dependence; or (d) a combination thereof.

Embodiment 77. The method of the foregoing Embodiments wherein the subject has previously been treated by a JAK inhibitor for the myeloproliferative neoplasm and has relapsed disease or is refractory to the JAK inhibitor.

Embodiment 78. The method of any of the foregoing Embodiments for treating a myeloproliferative neoplasm by inducing TSS50 in a human subject in need thereof, wherein the myeloproliferative neoplasm is treated by inducing TSS50 in the subject at a time point between and including 24 and 48 weeks.

Embodiment 79. The method of any of the foregoing Embodiments, comprising administering to the subject Compound (1) or a pharmaceutically acceptable salt thereof and ruxolitinib or momelotinib or a pharmaceutically acceptable salt thereof in amounts that induce no overlapping hematological toxicities selected from anemia, thrombocytopenia, and transfusion dependence.

Embodiment 80. The method of any of the foregoing Embodiments, comprising administering to the subject Compound (1) or a pharmaceutically acceptable salt thereof and ruxolitinib or momelotinib or a pharmaceutically acceptable salt thereof in amounts that induce no overlapping hematological toxicities in terms of anemia and thrombocytopenia and transfusion dependence.

Embodiment 81. The method of any of the foregoing Embodiments, for treating a myeloproliferative neoplasm by inducing a probability of SVR35 in the subject≥15% or 20%, wherein the subject has previously been treated by a JAK inhibitor for the myeloproliferative neoplasm and has relapsed disease or is refractory to the JAK inhibitor, wherein the treatment induces a probability of SVR35 in the human subject>15% or 20%.

Embodiment 81a. The method of any of the foregoing Embodiments, when undertaken as monotherapy, for treating a myeloproliferative neoplasm by inducing a probability of SVR35 in the subject≥20% or 25%, wherein the subject has previously been treated by a JAK inhibitor for the myeloproliferative neoplasm and has relapsed disease or is refractory to the JAK inhibitor, wherein the treatment induces a probability of SVR35 in the human subject>20% or 25%.

Embodiment 81b. The method of any of the foregoing Embodiments, when undertaken in combination with momelotinib, for treating a myeloproliferative neoplasm by inducing a probability of SVR35 in the subject≥20% or 30%, wherein the subject has previously been treated by a JAK inhibitor for the myeloproliferative neoplasm and has relapsed disease or is refractory to the JAK inhibitor, wherein the treatment induces a probability of SVR35 in the human subject>20% or 30%.

Embodiment 81c. The method of any of the foregoing Embodiments, when undertaken in combination with ruxolitinib, for treating a myeloproliferative neoplasm by inducing a probability of SVR35 in the subject≥40% or 50%, wherein the treatment induces a probability of SVR35 in the human subject>40% or 50%.

Embodiment 82. The method of any of the foregoing Embodiments, for treating a myeloproliferative neoplasm by inducing a probability of TSS50 in the subject≥30%, 35%, 40%, 45%, or 50%, wherein the subject has previously been treated by a JAK inhibitor for the myeloproliferative neoplasm and has relapsed disease or is refractory to the JAK inhibitor, wherein the treatment induces a probability of TSS50 in the human subject≥30%, 35%, 40%, 45%, or 50%.

Embodiment 82a. The method of any of the foregoing Embodiments, when undertaken as monotherapy, for treating a myeloproliferative neoplasm by inducing a probability of TSS50 in the subject≥30% or 40%, wherein the subject has previously been treated by a JAK inhibitor for the myeloproliferative neoplasm and has relapsed disease or is refractory to the JAK inhibitor, wherein the treatment induces a probability of TSS50 in the human subject≥30% or 40%.

Embodiment 82b. The method of any of the foregoing Embodiments, when undertaken in combination with momelotinib, for treating a myeloproliferative neoplasm by inducing a probability of TSS50 in the subject≥40% or 45%, wherein the subject has previously been treated by a JAK inhibitor for the myeloproliferative neoplasm and has relapsed disease or is refractory to the JAK inhibitor, wherein the treatment induces a probability of TSS50 in the human subject≥40% or 45%.

Embodiment 82c. The method of any of the foregoing Embodiments, when undertaken in combination with ruxolitinib, for treating a myeloproliferative neoplasm by inducing a probability of TSS50 in the subject≥50% or 60%, wherein the subject has previously been treated by a JAK inhibitor for the myeloproliferative neoplasm and has relapsed disease or is refractory to the JAK inhibitor, wherein the treatment induces a probability of TSS50 in the human subject≥50% or 60%.

Embodiment 83. The method of any of the foregoing Embodiments, wherein the subject has previously been treated by a JAK inhibitor for the myeloproliferative neoplasm and has relapsed disease or is refractory to the JAK inhibitor.

Embodiment 84. The method of any of the foregoing Embodiments, wherein the subject has previously been treated by a JAK inhibitor for the myeloproliferative neoplasm for ≥3, 6, 9, or 12 months and has relapsed disease or is refractory to the JAK inhibitor.

Embodiment 85. The method of any of the foregoing Embodiments, by inducing SVR35 and TSS50 in the subject, wherein the myeloproliferative neoplasm is treated by inducing SVR35 and TSS50 in the subject, optionally at a time point between and including 24 and 48 weeks.

Embodiment 86. The method of any of the foregoing Embodiments, by inducing a clinically meaningful response in the subject selected from: (a) a clinically meaningful decrease in splenomegaly from a baseline spleen size of, e.g., ≥450, 600, 750, 1,000, 1,500, or 2,000 cm³ by imaging; (b) a clinically meaningful reduction in an elevated TSS from a baseline TSS of, e.g., ≥15, 20, 25, 30, 35, 40, 45, or 50 per the Myelofibrosis Symptom Assessment Form (MFSAF) version 4.0; (c) a clinically meaningful improvement in poor bone marrow function selected from thrombocytopenia (e.g. a platelet count of ≤150, 100, 75, 50, or 25×10⁹/L), anemia (e.g. a Hgb level≤10, 9, or 8 g/dL), or transfusion dependence, or a combination thereof, (d) a clinically meaningful reduction in fibrosis from a baseline fibrosis of, e.g., grade 2 or higher; (e) a clinically meaningful increase in overall survival probability at, e.g., 1, 2, or 3 years; and (f) combinations thereof, and wherein the clinically meaningful response is induced in response to the administration.

Embodiment 87. The method of any of the foregoing Embodiments, by inducing a clinically meaningful decrease in splenomegaly (e.g. SVR35) from a baseline spleen size of, e.g., ≥450, 600, 750, 1,000, 1,500, or 2,000 cm³ by imaging, wherein the clinically meaningful decrease is induced in response to the administration.

Embodiment 88. The method of any of the foregoing Embodiments, by inducing a clinically meaningful reduction in an elevated TSS (e.g. TSS50) from a baseline TSS of, e.g., ≥15, 20, 25, 30, 35, 40, 45, or 50 per the Myelofibrosis Symptom Assessment Form (MFSAF) version 4.0, wherein the clinically meaningful reduction is induced in response to the administration.

Embodiment 89. The method of any of the foregoing Embodiments, by inducing a clinically meaningful reduction in fibrosis (e.g. ≥1 grade) from a baseline fibrosis of, e.g., grade 2 or higher, wherein the clinically meaningful reduction is induced in response to the administration.

Embodiment 90. The method of any of the foregoing Embodiments, by inducing a clinically meaningful increase in overall survival probability at, e.g., 1, 2, or 3 years, wherein the clinically meaningful increase is induced in response to the administration.

Embodiment 91. The method of any of the foregoing Embodiments, by inducing a clinically meaningful response in the subject selected from: (a) complete remission; (b) partial remission; (c) clinical improvement; and (d) stable disease; wherein the clinically meaningful response is evaluated by IWG-MRT response criteria; and the clinically meaningful response is induced in the subject in response to the administration.

Embodiment 92. The method of any of the foregoing Embodiments, wherein the subject at baseline has splenomegaly (e.g., ≥450, 600, 750, 1,000, 1,500, or 2,000 cm³ by imaging).

Embodiment 93. The method of any of the foregoing Embodiments, wherein the subject at baseline has elevated TSS (e.g., ≥15, 20, 25, 30, 35, 40, 45, or 50 per the Myelofibrosis Symptom Assessment Form (MFSAF) version 4.0).

Embodiment 94. The method of any of the foregoing Embodiments, wherein the subject at baseline has fibrosis (e.g., grade 2 or higher).

Embodiment 95. The method of any of the foregoing Embodiments, wherein the subject at baseline has poor bone marrow function.

Embodiment 96. The method of any of the foregoing Embodiments, wherein the subject at baseline has ≥2 or 3 measurable symptoms of myelofibrosis per MFSAF v4.

Embodiment 97. The method of any of the foregoing Embodiments, wherein the subject has previously been treated by a BET inhibitor (e.g. pelabresib), an ACVR1 inhibitor (e.g. pacritinib), or a telomerase inhibitor (e.g. imetelstat), optionally for ≥3 or 6 months.

Embodiment 98. The method of any of the foregoing Embodiments, wherein the myeloproliferative neoplasm is Intermediate-1, Intermediate-2, or High Risk myelofibrosis on the DIPSS scoring system.

Embodiment 99. The method of any of the foregoing Embodiments, wherein the myeloproliferative neoplasm is Low Risk myelofibrosis on the DIPSS scoring system.

Embodiment 100. The method of any of the foregoing Embodiments, wherein the subject is positive for a mutation selected from JAK2V617F, MPLW515L, or CALR, or a combination of two or three of the mutations.

Embodiment 101. The method of any of the foregoing Embodiments, further comprising monitoring the subject's cytokine levels and: (a) adjusting the dose of Compound (1) or a pharmaceutically acceptable salt thereof based on changes in the subject's cytokine levels; and/or (b) correlating changes in the cytokine levels with improvements in the subject's fibrosis.

Embodiment 102. The method of any of the foregoing Embodiments, wherein the Compound (1) or a pharmaceutically acceptable salt thereof is administered as monotherapy for the myeloproliferative neoplasm and the subject has previously been treated with a JAK inhibitor.

Embodiment 103. The method of any of Embodiments 1-101, wherein the Compound (1) or a pharmaceutically acceptable salt thereof is administered in combination with momelotinib or a pharmaceutically acceptable salt thereof, wherein the subject has previously been treated with a JAK inhibitor other than momelotinib or a pharmaceutically acceptable salt thereof and has poor bone marrow functioning.

Embodiment 104. The method of any of Embodiments 1-101, wherein the Compound (1) or a pharmaceutically acceptable salt thereof is administered in combination with ruxolitinib or a pharmaceutically acceptable salt thereof, wherein the subject is treatment naïve to a JAK inhibitor prior to initiating the method.

Embodiment 105. The method of any of Embodiments 1-101, wherein the Compound (1) or a pharmaceutically acceptable salt thereof is administered in combination with ruxolitinib or a pharmaceutically acceptable salt thereof in a subject who has been on ruxolitinib or a pharmaceutically acceptable salt thereof for ≥6 months and on a stable dose for at least 8 weeks, and who has either a suboptimal or plateau in response or lost response to ruxolitinib or a pharmaceutically acceptable salt thereof.

Embodiment 106. The method of any of the foregoing Embodiments, further comprising administering a BET inhibitor (e.g. pelabresib), an ACVR1 inhibitor (e.g. pacritinib), or a telomerase inhibitor (e.g. imetelstat).

Embodiment 107. The method of any of the foregoing Embodiments, optionally comprising administering the Compound (1) or a pharmaceutically acceptable salt thereof. BID.

Embodiment 108: The method of any one of the foregoing embodiments, comprising continuing administering to the subject a therapeutically effective amount of Compound (1) or the pharmaceutically acceptable salt,
wherein:
   the subject has a platelet count reduction of (a) less than 1 grade, or (b) less than or equal to 25×10⁹/L, and/or (c) less than or equal to 35%, and
   the platelet count reduction is determined by a change in the baseline platelet count determined within one week prior to administering Compound (1) or the pharmaceutically acceptable salt and a second platelet count determined at least 2 weeks after administering.

Embodiment 109: The method of any one of the foregoing embodiments, wherein the method minimizes, lessens, or ameliorates adverse events in the subject.

Embodiment 110: The method of any one of the foregoing embodiments, wherein the method results in an improvement in symptoms selected from the group consisting of fever, night sweats, early satiety, weight loss, fatigue, weakness, shortness of breath, enlarged spleen or liver, left upper abdominal pain, bone or joint pain, easy bruising, easy bleeding, and combinations of two or more thereof.

Embodiment 111: The method of any one of the foregoing embodiments, wherein the method results in minimizing, lessening, or ameliorating of fever, night sweats, early satiety, weight loss, fatigue, weakness, shortness of breath, enlarged spleen or liver, left upper abdominal pain, bone or joint pain, easy bruising, easy bleeding, or a combination of two or more thereof.

Embodiment 112: The method of any one of the foregoing embodiments, wherein the method results in a down-regulation of adiponectin, angiopoietin 1, angiotension II, beta-2 microglobulin, bone morphogenetic protein (BMP) and subtypes thereof (e.g., BMP1, BMP6, and BMP7), bone morphogenetic protein Rcp2, brain-derived neurotrophic factor (BDNF), C-C motif chemokine ligand (CCL) and subtypes thereof (e.g., CCL14 and CCL21), cluster of differentiation 40 ligand (CD40L), cluster of differentiation 40 (CD40), colony stimulating factor (CSF1), complement factor D, C-reactive protein (CRP), C-X3-C motif chemokine receptor 1 (CX3CR1), C-X-C motif chemokine ligand (CXL) and subtypes thereof (e.g., CXL11, CXCL12, and CXCL16), Dickkopf WNT Signaling Pathway Inhibitor 1 (DKK1), endoglin, eotaxin and subtypes thereof (e.g., eotaxin-2 and eotaxin-3), epidermal growth factor (EGF), epidermal growth factor receptor (EGFR), erythropoietin (EPO), extracellular matrix protein tenascin-C (TN-C), extracellular newly identified receptor for advanced glycation end-products binding protein (EN-RAGE), ferritin, fibroblast growth-factor (FGF), Fms Related Receptor Tyrosine Kinase 3 (FLT3), Fms-related tyrosine kinase 3 ligand (FLT-3L), granulocyte-macrophage colony-stimulating factor (GM-CSF), growth colony stimulating factor (G-CSF), growth-regulated protein alpha (GRO-alpha), heparin-binding EGF-like growth factor (HB-EGF), hepatocyte growth factor (HGF), insulin like growth factor binding protein 2 (IGFBP2), intercellular adhesion molecule 1 (ICAM-1), interferon (IFN) and subtypes thereof (e.g., interferon gamma-inducible protein 9 (IP-9), interferon-alpha (IFN-alpha), interferon-gamma (IFNg), interferon-gamma inducible protein (IFN-g-IP), interferon-gamma inducible protein 10 (IP-10), interleukin (IL) and subfactors thereof (e.g., IL-1a, IL-1Ra IL-2, IL-2R, IL-2Ra, IL-4, IL-5, IL-5a IL-6, IL-7, IL-8, IL-10, IL-11, IL-12, IL-12p40, IL-12p70, IL-13, IL-15, IL-17, IL-17A, IL-17F, IL-18, IL-22, IL-23, IL-1B, and IL-1RA), isocitrate dehydrogenase 1 (IDH1), isocitrate dehydrogenase 2 (IDH2), latency-associated peptide transforming growth factor-β1 (LAP TGF-b1), leptin (LEP), LIF interleukin 6 family cytokine (LIF), macrophage colony stimulating factor 1 (MCSF), macrophage derived chemokine (MDC), macrophage inflammatory protein and subtypes thereof (e.g., MIP-1, MIP-1b, MIP-1a, MIP-3a, and MIP-3b), macrophage-derived chemokine (MDC), matrix metalloproteinase and subtypes thereof (e.g., MMP-3 and MMP-9), monocyte chemotactic protein and subtypes thereof (e.g., MCP-1, MCP-2, MCP-3, MCP-4, and MCP-5), monokine-induced by gamma (MIG), myelin basic protein and subtypes thereof (e.g., MBP R2 and MBP7), myeloperoxidase (MPO), myoglobin, osteoprotegerin (OPG), pentraxin (PTX) and subtypes thereof (e.g., PTX3), plasma protein factor VII (factor VII), plasminogen activator inhibitor-1 (PAI1), platelet factor 4 (PF-4), platelet-derived growth factor BB (PDGF-BB), pulmonary and activation-regulated chemokine (PARC), regulated on activation normal T cell expressed and secreted (RANTES), stem cell factor (SCF), ten-eleven translocation 2 (TET2), thrombopoietin (TPO), thymus and activation-regulated chemokine (TARC), tissue inhibitor of metalloproteinase-1 (TIMP1), transforming growth factor-β (TGF-beta), transforming growth factor-β1 (TGF-b1), tumor necrosis factor and subtypes thereof (TNF-1, TNF-a, TNF-RI, and TNF-RII), urokinase plasminogen activator receptor (uPAR), vascular adhesion molecule (VCAM1), vascular endothelial growth factor (VEGF) and subtypes thereof (VEGFb), vascular endothelial growth factor receptor (VGFR), α2-macroglobulin (A2M), or a combination of two or more thereof.

Embodiment 113: The method of any one of the foregoing embodiments, wherein the method results in a down-regulation of IL-1Ra, IL-6, IL-10, IL-12, IL-12, TGF-beta, EGFR, ferritin, GRO-a, MMP-9, PAI-1, RANTES, TIMP-1, TNFR-2, VCAM-1, or a combination of two or more thereof.

Embodiment 114: The method of any one of the foregoing embodiments, wherein the method improves clinically meaningful overall survival in the subject.

Embodiment 115: The method of any one of the foregoing embodiments, wherein the method results in the subject exhibiting normalization or increased platelet count, normalization or increased neutrophil count, normalization or increased hemoglobin count, transfusion improvement (including conversion into transfusion independence), or a combination of two or more thereof.

Embodiment 116: The method of any one of the foregoing embodiments, wherein the method results in the subject exhibiting bone marrow fibrosis reduction, total symptom reduction, cytokine modulation, or a combination of two or more thereof.

Embodiment 117: The method of any one of the foregoing embodiments, wherein the method results in a Total Symptoms Score (TSS) reduction of greater than 50%.

Embodiment 119: The method of any one of the foregoing embodiments, wherein the method results in a Total Symptoms Score (TSS) reduction of greater than 70%.

Embodiment 120: The method of any one of the foregoing embodiments, wherein the method reduces bone marrow fibrosis in the subject.

Embodiment 121: The method of any one of the foregoing embodiments, wherein the method results in at least 1 grade reduction of bone marrow fibrosis in the subject.

Embodiment 122: The method of any one of the foregoing embodiments, wherein the method reduces spleen volume in the subject.

Embodiment 123: The method of any one of the foregoing embodiments, wherein the method results in clinical improvement of blood cells or neutropenia counts, and/or ameliorating thrombocytopenia.

Embodiment 124: The method of any one of the foregoing embodiments, wherein the method results in the subject being cytogenetic remission or molecular partial or complete remission.

Embodiment 125: The method of any one of the foregoing embodiments, wherein the method results in partial or complete remission in the subject.

Embodiment 126: The method of any one of the foregoing embodiments, wherein the method ameliorates anemia in the subject.

Embodiment 127: The method of any one of the foregoing embodiments, wherein the method results in ≥20 g/L increasing in hemoglobin level or changing transfusion-depending to transfusion-independent in the subject.

Embodiment 128: The method of any one of the foregoing embodiments, wherein the method results in improvement of Patient Global Impression of Change (PGIC) questionnaire (at least 1 score increase) at week 24.

Embodiment 129: The method of any one of the foregoing embodiments, wherein Compound (1) or a pharmaceutically acceptable salt thereof is administered in an amount from about 120 mg to about 3000 mg.

Embodiment 130: The method of any one of the foregoing embodiments, wherein Compound (1) or a pharmaceutically acceptable salt thereof is administered in an amount from about 240 mg to about 1440 mg daily.

Embodiment 131: The method of any one of the foregoing embodiments, wherein Compound (1) or a pharmaceutically acceptable salt thereof is administered in an amount from about 240 mg to about 1200 mg.

Embodiment 132: The method of any one of the foregoing embodiments, wherein Compound (1) or a pharmaceutically acceptable salt thereof is administered in an amount from about 240 mg to about 1000 mg.

Embodiment 133: The method of any one of the foregoing embodiments, the therapeutically effective amount of Compound (1) or the pharmaceutically acceptable salt is from about 240 mg to about 900 mg.

Embodiment 134: The method of any one of the foregoing embodiments, wherein Compound (1) or a pharmaceutically acceptable salt thereof is administered in an amount from about 240 mg to about 800 mg.

Embodiment 135: The method of any one of the foregoing embodiments, wherein Compound (1) or a pharmaceutically acceptable salt thereof is administered in an amount from about 240 mg to about 700 mg.

Embodiment 136: The method of any one of the foregoing embodiments, wherein Compound (1) or a pharmaceutically acceptable salt thereof is administered in an amount from about 240 mg to about 600 mg.

Embodiment 137: The method of any one of the foregoing embodiments, wherein Compound (1) or a pharmaceutically acceptable salt thereof is administered in an amount from about 240 mg to about 500 mg.

Embodiment 138: The method of any one of the foregoing embodiments, wherein Compound (1) or a pharmaceutically acceptable salt thereof is administered in an amount from about 350 mg to about 1350 mg.

Embodiment 139: The method of any one of the foregoing embodiments, wherein Compound (1) or a pharmaceutically acceptable salt thereof is administered in an amount from about 400 mg to about 1250 mg.

Embodiment 140: The method of any one of the foregoing embodiments, wherein the therapeutically effective amount of Compound (1) or the pharmaceutically acceptable salt from about 450 mg to about 1150 mg.

Embodiment 141: The method of any one of the foregoing embodiments, wherein Compound (1) or a pharmaceutically acceptable salt thereof is administered in an amount from about 500 mg to about 1050 mg.

Embodiment 142: The method of any one of the foregoing embodiments, wherein Compound (1) or a pharmaceutically acceptable salt thereof is administered in an amount from about 550 mg to about 1000 mg.

Embodiment 143: The method of any one of embodiments 1-128, wherein Compound (1) or a pharmaceutically acceptable salt thereof is administered in an amount from about 350 mg to about 1000 mg.

Embodiment 144: The method of any one of embodiments 1-128, wherein Compound (1) or a pharmaceutically acceptable salt thereof is administered in an amount from about 450 mg to about 900 mg.

Embodiment 145: The method of any one of embodiments 1-128, wherein Compound (1) or a pharmaceutically acceptable salt thereof is administered in an amount from about 450 mg to about 800 mg.

Embodiment 146: The method of any one of embodiments 1-128, wherein Compound (1) or a pharmaceutically acceptable salt thereof is administered in an amount from about 450 mg to about 750 mg.

Embodiment 147: The method of any one of embodiments 1-128, wherein Compound (1) or a pharmaceutically acceptable salt thereof is administered in an amount from about 470 mg to about 730 mg.

Embodiment 148: The method of any one of embodiments 1-128, wherein Compound (1) or a pharmaceutically acceptable salt thereof is administered in an amount from about 120 mg to about 580 mg, about 581 mg to about 1040 mg, or about 1041 mg to about 1500 mg.

Embodiment 149: The method of any one of embodiments 1-128, wherein Compound (1) or a pharmaceutically acceptable salt thereof is administered in an amount from about 120 mg/day to about 1500 mg/day.

Embodiment 150: The method of any one of embodiments 1-128, wherein Compound (1) or a pharmaceutically acceptable salt thereof is administered in an amount from about 350 mg/day to about 2100 mg/day.

Embodiment 151: The method of any one of embodiments 1-128, wherein Compound (1) or a pharmaceutically acceptable salt thereof is administered in an amount from about 550 mg/day to about 2000 mg/day.

Embodiment 152: The method of any one of embodiments 1-128, wherein Compound (1) or a pharmaceutically acceptable salt thereof is administered in an amount from about 700 mg/day to about 1950 mg/day.

Embodiment 153: The method of any one of embodiments 1-128, wherein Compound (1) or a pharmaceutically acceptable salt thereof is administered in an amount from about 120 mg/day to about 800 mg/day.

Embodiment 154: The method of any one of embodiments 1-128, wherein Compound (1) or a pharmaceutically acceptable salt thereof is administered in an amount from about 200 mg/day to about 750 mg/day.

Embodiment 155: The method of any one of embodiments 1-128, wherein Compound (1) or a pharmaceutically acceptable salt thereof is administered in an amount from about 250 mg/day to about 650 mg/day.

Embodiment 156: The method of any one of embodiments 1-128, wherein Compound (1) or a pharmaceutically acceptable salt thereof is administered in an amount from about 240 mg to about 1440 mg twice daily.

Embodiment 157: The method of any one of embodiments 1-128, wherein Compound (1) or a pharmaceutically acceptable salt thereof is administered in an amount of about 1440 mg twice daily.

Embodiment 158: The method of any one of the foregoing embodiments, comprising administering the selective PIM1 inhibitor or Compound (1) or its pharmaceutically acceptable salt at least once daily.

Embodiment 159: The method of any one of the foregoing embodiments, comprising administering the selective PIM1 inhibitor or Compound (1) or its pharmaceutically acceptable salt at least twice daily.

Embodiment 160: The method of any one of the foregoing embodiments, comprising administering the selective PIM1 inhibitor or Compound (1) or its pharmaceutically acceptable salt twice daily.

Embodiment 161: The method of any one of the foregoing embodiments, comprising administering the selective PIM1 inhibitor or Compound (1) or its pharmaceutically acceptable salt at least three times daily.

Embodiment 162: The method of any one of the foregoing embodiments, comprising administering the selective PIM1 inhibitor or Compound (1) or its pharmaceutically acceptable salt at least four times daily.

Embodiment 163: The method of any one of the foregoing embodiments, further comprising administering ruxolitinib or its pharmaceutically acceptable salt in an amount of about 5 mg, 10 mg, 15 mg, or 25 mg.

Embodiment 164: The method of any one of the foregoing embodiments, further comprising administering fedratinib or its pharmaceutically acceptable salt in an amount of about 200 mg or 400 mg.

Embodiment 165: The method of any one of the foregoing embodiments, further comprising administering pacritinib or its pharmaceutically acceptable salt in an amount of about 100 mg or 200 mg.

Embodiment 166: The method of any one of the foregoing embodiments, further comprising administering momelotinib or its pharmaceutically acceptable salt in an amount of about 100 mg or 200 mg.

Embodiment 167: The method of any one of the foregoing embodiments, further comprising administering luspatercept in an amount of at least about 1 mg/kg or 1.25 mg/kg based on the subject's body weight.

Embodiment 168: The method of embodiment 167, wherein luspatercept is administrated once every 3 weeks by subcutaneous injection for at least 21 weeks.

Embodiment 169: The method of any one of embodiments 163-166, wherein administering ruxolitinib, fedratinib, pacritinib, or momelotinib, or the pharmaceutically acceptable salt is at least one dose a day.

Embodiment 170: The method of any one of embodiments 163-166, wherein administering ruxolitinib, fedratinib, pacritinib, or momelotinib, or the pharmaceutically acceptable salt is at least two doses a day.

Embodiment 171: The method of any one of embodiments 163-168, comprising administering Compound (1) or the pharmaceutically acceptable salt and ruxolitinib, fedratinib, pacritinib, momelotinib, luspatercept, ropeginterferon alfa-2b, or navtemadelin, or the pharmaceutically acceptable salt thereof concurrently.

Embodiment 172: The method of any one of embodiments 163-168, comprising administering Compound (1) or the pharmaceutically acceptable salt and ruxolitinib, fedratinib, pacritinib, momelotinib, luspatercept, ropeginterferon alfa-2b, or navtemadelin, or the pharmaceutically acceptable salt is sequentially.

Embodiment 173: The method of any one of the foregoing embodiments, wherein, when a baseline platelet count is referenced, the baseline platelet count is measured within one week prior to initiation of the administering Compound (1) or the pharmaceutically acceptable salt.

Embodiment 174: The method of any one of the foregoing embodiments, further comprising monitoring complete blood counts in the subject and titrating a dose amount of Compound (1) or a pharmaceutically acceptable salt thereof.

Embodiment 175: The method of any one of the foregoing embodiments, further comprising monitoring platelet counts and increasing the dose amount or frequency (i.e., up-titrating) if the platelet count change is (a) less than 1 grade, (b) decreased by less than 25×10⁹/L, and/or (c) decreased by less than 35%, based on the subject's baseline platelet count (i.e., platelet count prior to starting treatment).

Embodiment 176: The method of any one of the foregoing embodiments, further comprising monitoring platelet counts and decreasing the dose amount or lessening the dose frequency (collectively, down-titrating) if the platelet count change is (a) up 1 or more grades, (b) decreased by more than 25×10⁹/L, and/or (c) decreased by more than 35%.

Embodiment 177: The method of claim 175, wherein the platelet count has moved up from grade 1 to grade 2, 3, or 4, or from grade 2 to grade 3 or 4, or from grade 3 to grade 4.

Embodiment 178: The method of any one of embodiments 164-177, wherein the monitoring is performed prior to, during, and/or after the treatment.

Embodiment 179: The method of any one of embodiments 174-178, wherein the monitoring is performed once a week or every 2 to 4 weeks.

Embodiment 180: The method of any one of embodiments 174-179, further comprising continuing the titrating the dose amount or frequency until achieving a desired stable clinical state.

Embodiment 181: The method of any one of the foregoing embodiments, further comprising when ruxolitinib, fedratinib, pacritinib, momelotinib, luspatercept, ropeginterferon alfa-2b, or navtemadelin, or a pharmaceutically acceptable salt thereof is administered, monitoring complete blood counts in the subject and titrating the dose amount of ruxolitinib, fedratinib, pacritinib, momelotinib, luspatercept, ropeginterferon alfa-2b, or navtemadelin, or pharmaceutically acceptable salt thereof.

Embodiment 182: The method of any one of the foregoing embodiments, further comprising monitoring comprising assessing platelet counts in the subject prior to, during, and/or after the treatment.

Embodiment 183: The method of any one of the foregoing embodiments, further comprising, when ruxolitinib or its pharmaceutically acceptable salt is administered, starting ruxolitinib or its pharmaceutically acceptable salt at a dose:
20 mg twice daily if baseline platelet count is greater than 200×10⁹/L,
10 mg twice daily if baseline platelet count is from 100×10⁹/L to 200×10⁹/L,
5 mg twice daily if baseline platelet count is from 50×10⁹/L to less than 100×10⁹/L.

Embodiment 184: The method of embodiment 183, further comprising increasing the dose amount if the subject's response is insufficient and platelet and neutrophil counts are adequate.

Embodiment 185: The method of embodiment 184, comprising increasing the dose in the amount of 5 mg twice daily increments to a maximum of 25 mg twice daily.

Embodiment 186: The method of embodiment 184 or 185, comprising increasing the dose amount of ruxolitinib or a pharmaceutically acceptable salt thereof in the subject that meets all of the following conditions:
failure to achieve a reduction from pretreatment baseline in either palpable spleen length of 50% or a 35% reduction in spleen volume as measured by computed tomography (CT) or magnetic resonance imaging (MRI);
platelet count greater than 125×10⁹/L at 4 weeks and platelet count never below 100×10⁹/L; and
ANC Levels greater than 0.75×10⁹/L.

Embodiment 187: The method of embodiment 184 or 185, comprising increasing the dose amount of ruxolitinib or a pharmaceutically acceptable salt thereof in the subject by increments of 5 mg daily to a maximum of 10 mg twice daily if:
the platelet count has remained at least 40×10⁹/L,
the platelet count has not fallen by more than 20% in the prior 4 weeks, the ANC is more than 1×10⁹/L, and
the dose has not been reduced or interrupted for an adverse event or hematological toxicity in the prior 4 weeks.

Embodiment 188: The method of embodiment 184 or 185, comprising titrating the dose amount of ruxolitinib or a pharmaceutically acceptable salt thereof according to Table (a) for thrombocytopenia for the subject whose treatment started with a platelet count of 100×10⁹/L or greater:

TABLE (a)

| | Dose at Time of Platelet Decline | | | | |
|---|---|---|---|---|---|
| | 25 mg | 20 mg | 15 mg | 10 mg | 5 mg |
| | twice daily | twice daily | twice daily | twice daily | twice daily |
| Platelet Count | New Dose | New Dose | New Dose | New Dose | New Dose |
| 100 to less than | 20 mg | 15 mg | No Change | No Change | No Change |
| 125 × 10⁹/L | twice daily | twice daily | | | |
| 75 to less than | 10 mg | 10 mg | 10 mg | No Change | No Change |
| 100 × 10⁹/L | twice daily | twice daily | twice daily | | |
| 50 to less than | 5 mg | 5 mg | 5 mg | 5 mg | No Change |
| 75 × 10⁹/L | twice daily | twice daily | twice daily | twice daily | |
| Less than 50 × 10⁹/L | Hold | Hold | Hold | Hold | Hold |

Embodiment 189: The method of embodiment 184 or 185, comprising titrating the dose amount of ruxolitinib or a pharmaceutically acceptable salt thereof according to Table (b) for thrombocytopenia for the subject whose treatment started with a platelet count of 50×10⁹/L to less than 100×10⁹/L:

TABLE (b)

| Platelet Count | Dosing Recommendations |
|---|---|
| Less than 25 × 10⁹/L | Interrupt dosing. |
| 25 × 10⁹/L to less than 35 × 10⁹/L | Decrease dose by 5 mg once daily. |
| AND the platelet count decline is | For patients on 5 mg once daily, |
| less than 20% during the prior | maintain dose at 5 mg once daily. |
| four weeks | |
| 25 × 10⁹/L to less than 35 × 10⁹/L | Decrease dose by 5 mg twice daily. |
| AND the platelet count decline is | For patients on 5 mg twice daily, |
| 20% or greater during the prior | decrease the dose to 5 mg once daily. |
| four weeks | For patients on 5 mg once daily, |
| | maintain dose at 5 mg once daily. |

Embodiment 190: The method of any one of embodiments 1-182, further comprising, when fedratinib is administered, starting fedratinib or a pharmaceutically acceptable salt thereof in the subject at 200, 300, or 400 mg once daily for patients with a baseline platelet count of greater than or equal to 50×10⁹/L.

Embodiment 191: The method of embodiment 190, comprising reducing the dose amount of fedratinib to 100 mg or below, or temporarily stopping the doses if adverse reactions develop.

Embodiment 192: The method of embodiment 191, wherein the adverse reaction is anemia, grade 4 thrombocytopenia, grade 3 thrombocytopenia with active bleeding, grade 3 or higher nausea, vomiting, diarrhea, anemia, or elevations of alanine transaminase (ALT), aspartate aminotransferase (AST), or bilirubin.

Embodiment 193: The method of any of embodiments 1-182, further comprising, when pacritinib is administered, starting pacritinib or a pharmaceutically acceptable salt thereof in the subject at 100 mg or 200 mg twice daily for patients with a baseline platelet count below 50×10⁹/L.

Embodiment 184: The method of embodiment 183, comprising reducing the dose amount of pacritinib to 100 mg or below if adverse reactions develop.

Embodiment 195: The method of embodiment 194, wherein the adverse reaction is diarrhea, thrombocytopenia, nausea, anemia, and peripheral edema.

Embodiment 196: The method of embodiment 194 or 195, further comprising temporarily stopping the dose if any clinically significant worsening of thrombocytopenia that lasts more than 7 days, until toxicity is resolved, and restarting pacritinib or the pharmaceutically acceptable salt at 50% of last dose given.

Embodiment 197: The method of any of embodiments 1-182, further comprising, when luspatercept is administered, starting luspatercept in the subject at 0.5 mg/kg or 1 mg/kg by subcutaneous injection once every 3 weeks.

Embodiment 198: The method of embodiment 197, comprising titrating the dose amount of luspatercept if the subject's response is insufficient or adverse actions develop.

Embodiment 199: The method of embodiment 198, further comprising titrating the dose based on responses according to Table (c):

TABLE (c)
Dose Increases for Insufficient Response at Initiation of Treatment

| | |
|---|---|
| No reduction in RBC transfusion burden after at least 2 | Increase the dose to 1.25 mg/kg every |
| consecutive doses (6 weeks) at the 1 mg/kg starting dose | 3 weeks |
| No reduction in RBC transfusion burden after 3 | Discontinue treatment |
| consecutive doses (9 weeks) at 1.25 mg/kg | |

Embodiment 200: The method of embodiment 198, comprising titrating the dose based on responses according to Table (d):

TABLE (d)
Dose Modifications for Predose Hemoglobin
Levels or Rapid Hemoglobin Rise

| | |
|---|---|
| Predose hemoglobin is greater than | Interrupt treatment |
| or equal to 11.5 g/dL in the absence | Restart when the hemoglobin |
| of transfusions | is no more than 11 g/dL |
| Increase in hemoglobin greater than | |
| 2 g/dL within 3 weeks in the absence | |
| of transfusions and | |
| current dose is 1.25 mg/kg | Reduce dose to 1 mg/kg |
| current dose is 1 mg/kg | Reduce dose to 0.8 mg/kg |
| current dose is 0.8 mg/kg | Reduce dose to 0.6 mg/kg |
| current dose is 0.6 mg/kg | Discontinue treatment |

Embodiment 201: The method of any of embodiment 1-182, further comprising, when momelotinib is administered, administering momelotinib or a pharmaceutically acceptable salt thereof in the subject at a dose of about 50 mg to about 200 mg when the subject is suffering from intermediate or high-risk primary or secondary (post-PV or post-ET) myelofibrosis with hemoglobin level<10.

Embodiment 202: The method of any one of the foregoing embodiments, wherein the myeloproliferative neoplasm is responsive to selective PIM1 inhibition.

Embodiment 203: The method of any one of the foregoing embodiments, wherein the myeloproliferative neoplasm is a JAK inhibitor-resistant myeloproliferative neoplasm.

Embodiment 204: The method of any one of the foregoing embodiments, wherein the myeloproliferative neoplasm is myelofibrosis.

Embodiment 205: The method of any one of embodiments 1-203, wherein the myeloproliferative neoplasm is intermediate-risk myelofibrosis or high-risk myelofibrosis.

Embodiment 206: The method of any one of the embodiments 1-203, wherein the myeloproliferative neoplasm is primary or idiopathic myelofibrosis.

Embodiment 207: The method of any one of embodiments 1-203, wherein the myeloproliferative neoplasm is secondary myelofibrosis. (e.g., post-PV myelofibrosis and post-ET myelofibrosis).

Embodiment 208: The method of any one of embodiments 1-203, wherein the myeloproliferative neoplasm is selected from polycythemia vera (PV), primary or essential thrombocythemia (ET), or pre-myelofibrosis.

Embodiment 209: The method of any one of the foregoing embodiments, wherein the subject has not been previously treated with a JAK inhibitor.

Embodiment 210: The method of any one of the foregoing embodiments, wherein the subject has not been treated with a JAK inhibitor for at least 2 weeks prior to administering Compound (1) or the pharmaceutically acceptable salt.

Embodiment 211: The method of any one of the foregoing embodiments wherein the subject has been previously treated with a JAK inhibitor and exhibited an inadequate response.

Embodiment 212: The method of any one of the foregoing embodiments, wherein the subject has been previously treated with a JAK inhibitor and exhibited an inadequate response defined as resistance or intolerance to the JAK inhibitor, loss or failure to obtain significant spleen response or total symptom response, and/or developing clinically significant reduction of count of blood cells, neutropenia, or thrombocytopenia.

Embodiment 213: The method of any one of the foregoing embodiments, wherein the subject is intolerant of a JAK inhibitor therapy, is resistant to a JAK inhibitor therapy, or is ineligible for a JAK inhibitor therapy.

Embodiment 214: The method of any one of the foregoing embodiments, wherein the subject has experienced relapse from a prior JAK inhibitor therapy.

Embodiment 215: The method of any one of the foregoing embodiments, wherein the subject has experienced relapse from a prior bone marrow transplant.

Embodiment 216: The method of any one of the foregoing embodiments, wherein the subject has moderate or severe splenomegaly at baseline.

Embodiment 217: The method of any one of the foregoing embodiments, wherein the subject has a total symptoms score (TSS) at baseline greater than 15, 20, 25, 30, 35, 40, 45, or 50.

Embodiment 218: The method of any one of the foregoing embodiments, wherein the subject is in need of cytokine modulation.

Embodiment 219: The method of any one of the foregoing embodiments, wherein the subject has a JAK2V16F mutation.

Embodiment 220: The method of any one of the foregoing embodiments, wherein the subject has a CALR mutation.

Embodiment 221: The method of any one of the foregoing embodiments, wherein the subject has grade 2 or higher fibrosis.

Embodiment 222: The method of any one of the foregoing embodiments, wherein the subject has an absolute neutrophil count of greater than 1×10⁹/L.

Embodiment 223: The method of any one of the foregoing embodiments, wherein the subject has a peripheral blood blast less than 10%.

Embodiment 224: The method of any one of the foregoing embodiments, wherein the subject has a baseline platelet count of less than 200×10⁹/L.

Embodiment 225: The method of any one of the foregoing embodiments, wherein the subject has a baseline platelet count of less than 150×10⁹/L.

Embodiment 226: The method of any one of the foregoing embodiments, wherein the subject has a baseline platelet count of less than 100×10⁹/L.

Embodiment 227: The method of any one of the foregoing embodiments, wherein the subject has a baseline platelet count of less than 50×10⁹/L.

Embodiment 228: The method of any one of the foregoing embodiments, wherein the subject has a baseline platelet count of greater than 10×10⁹/L.

Embodiment 229: The method of any one of the foregoing embodiments, wherein the subject has a baseline platelet count of greater than 25×10⁹/L.

Embodiment 230: The method of any one of the foregoing embodiments, wherein the subject has a baseline platelet count of greater than 35×10⁹/L.

Embodiment 231: The method of any one of the foregoing embodiments, wherein Compound (1) or its pharmaceutically acceptable salt is administered for a period from about one week to about two years.

Embodiment 232: The method of any one of the foregoing embodiments, wherein Compound (1) or its pharmaceutically acceptable salt is administered for a period from about 15 weeks to about one year.

Embodiment 233: The method of any one of the foregoing embodiments, wherein Compound (1) or its pharmaceutically acceptable salt is administered for a period from about 24 weeks to about one year.

Embodiment 234: The method of any one of the foregoing embodiments, wherein Compound (1) or the pharmaceutically acceptable salt is administered orally.

Embodiment 235: The method of any one of the foregoing embodiments, wherein a hydrochloric acid salt of Compound (1) is administered orally.

Embodiment 236: The method of any one of the foregoing embodiments, wherein a crystalline hydrochloric acid salt of Compound (1) is administered orally.

Embodiment 237: The method of any one of the foregoing embodiments, when a selective PIM1 inhibitor is administered, the selective PIM1 inhibitor has a PIM1/PIM-2 IC50 ratio of less than 0.05, 0.045, 0.04, 0.035, 0.03, or 0.025.

### EXAMPLES

### Example 1: In Vitro Hematological Tolerability of Compound (1), Ruxolitinib, Pacritinib, and Momelotinib Individually

The potential effects of Compound (1), ruxolitinib, pacritinib and momelotinib (collectively referred to as test compounds) on human myeloid, erythroid and megakaryocyte progenitor proliferation were evaluated using colony forming cell assays to generate IC₅₀ values.

Clonogenic progenitors of human erythroid (CFU-E, BFU-E), granulocyte-monocyte (CFU-GM), and multipotential (CFU-GEMM) lineages were assessed in a semi-solid methylcellulose-based media formulation containing rhIL-3 (10 ng/mL), rhGM-SCF (10 ng/mL), rhSCF (50 ng/mL), and Epo (3 U/mL).

Using 10 mM stock solutions of each test compound in DMSO, serial dilutions were prepared to achieve concentrations of 3.33, 1, 0.333, 0.1, 0.0333, 0.01, and 0.00333 mM (1000× higher than the desired test concentrations). When the compounds were added in at 1:1000 v/v the appropriate final test concentrations of 10, 3.33, 1, 0.333, 0.1, 0.0333, 0.01 and 0.00333 μM were achieved. In addition, 5-FU was evaluated at 1.0, 0.1 and 0.01 μg/mL as a positive control for toxicity.

Normal human bone marrow light density cells (lot #0210901), derived from normal bone marrow, (Lonza, Maryland) and qualified at ReachBio, were stored at −152° C. until required for the assay. On the day of the experiment, the cells were thawed rapidly, diluted in 10 mL of Iscove's modified Dulbecco's medium containing 10% fetal bovine serum (IMDM+10% FBS) and washed by centrifugation (approximately 1500 r.p.m. for 10 minutes at room temperature). The supernatant was discarded and the cell pellets were resuspended in a known volume of IMDM+10% FBS. A cell count (3% glacial acetic acid) and viability assessment (trypan blue exclusion test) was performed for the bone marrow sample.

Clonogenic progenitors of human megakaryocyte (CFU-Mk) were assessed in a collagen-based media formulation containing rhIL-3 (10 ng/mL), rhIL-6 (10 ng/mL), and Tpo (50 ng/mL). Clonogenic progenitors of the human erythroid (CFU-E and BFU-E), myeloid (CFU-GM), and megakaryocyte (CFU-Mk) lineages were set up in the media formulations described above. Individually, the test compounds were added to the medium to give the final desired concentrations (10, 3.33, 1, 0.333, 0.1, 0.0333, 0.01 and 0.00333 μM). 5-Fluorouracil (Sigma Aldrich) was used as a positive control for progenitor proliferation (inhibition of colony growth) and was introduced to the human bone marrow cultures at 1.0, 0.1, and 0.01 μg/mL. Solvent control cultures (containing no compound but 0.1% DMSO) as well as standard controls (containing no compound and no DMSO) were also initiated. Once introduced into the matrices, the tubes were vortexed to ensure an equal distribution throughout.

Human myeloid and erythroid progenitor assays were initiated at 1.2×10⁴ cells per culture (lot #0210921) and the human megakaryocyte assays were initiated at 1.2×10⁵ cells per culture (lot #0210921). Following 14 days in culture, erythroid and myeloid colonies were assessed microscopically in situ and scored by trained personnel. The colonies were divided into the following categories based on size and morphology: CFU-E, BFU-E, CFU-GM, and CFU-GEMM. The human megakaryocyte cultures were transferred from the 35 mm dishes to labeled glass slides, fixed with methanol/acetone fixative, and then stained with anti-human CD41 antibody and an alkaline phosphate detection system according to manufacturers' instructions. The colonies were assessed microscopically and scored by trained personnel and divided into the following categories based on size: CFU-Mk (3-20), CFU-Mk (21-49), and CFU-Mk (≥50).

Statistical analysis: The mean±standard deviation of three replicate cultures was calculated for both erythroid and myeloid progenitors. Two-tailed standard t-tests were performed to assess if there was a difference in the number of colonies generated between solvent control and treated cultures. Due to the potential subjectivity of colony enumeration, a p value of less than 0.01 is deemed significant. To calculate the concentration of 50% inhibition of colony growth (IC₅₀) for each compound, a dose response curve was generated plotting the log of the compound concentration versus the percentage of control colony growth using GraphPad Prism 9 software.

Erythroid (CFU-E and BFU-E) and myeloid (CFU-GM) colony enumeration was performed by trained personnel. In addition, the distribution of colony types as well as general colony and cellular morphology was analyzed. The variance in colony number detected in replicate cultures was representative of the historical coefficient of variation for colony enumeration using these types of assays. The number and distribution of colonies detected in the solvent control (0.1% DMSO for single agents) was no different from the standard control (containing no compound and no DMSO). For statistical analysis colony numbers in single compound treated cultures were compared to the 0.1% DMSO solvent control cultures. 5-FU was used as a positive control for toxicity and the inhibitory effects obtained for 5-FU were as expected.

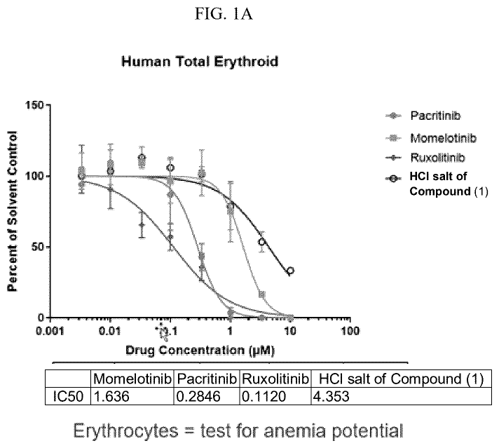

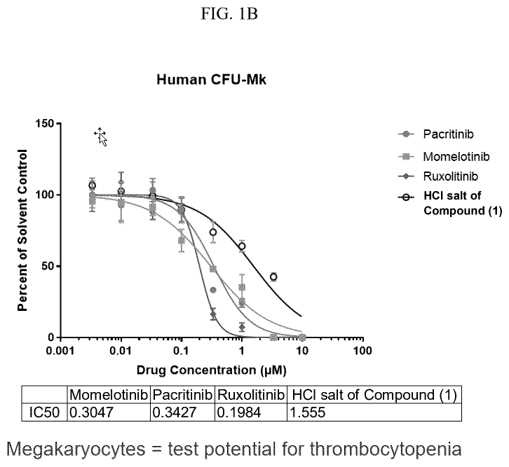

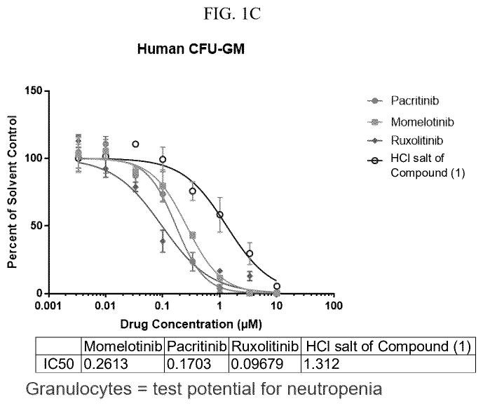

The effect of the individual test compounds in a methylcellulose-based medium on human erythroid progenitor proliferation are presented in FIG. 1A (IC₅₀ values are listed in the figure), on human megakaryocyte progenitor proliferation are presented in FIG. 1B (IC₅₀ values are listed in the figure), and on human myeloid progenitor proliferation are presented in FIG. 1C (IC₅₀ values are listed in the figure). All of the test compounds inhibited erythroid, megakaryocyte, and myeloid progenitor proliferation with IC₅₀ values ranging from 0.1-4.35 μM. Ruxolitinib was the most potent and Compound (1) was the least potent. Momelotinib significantly inhibited erythroid progenitor proliferation at 10 and 3.33 μM and additionally myeloid progenitor proliferation at 1.0 μM, which provided IC₅₀ values of 1.6 and 0.26 μM for erythroid and myeloid progenitor proliferation (FIG. 1A and FIG. 1C, respectively). Pacritinib significantly inhibited erythroid progenitor proliferation from 10-0.333 μM and additionally myeloid progenitor proliferation at 0.1 μM, which provided IC₅₀ values of 0.28 and 0.17 μM for erythroid and myeloid progenitor proliferation (FIGS. 1A and 1C, respectively). Ruxolitinib significantly inhibited both erythroid and myeloid progenitor proliferation from 10-0.033 μM, which provided IC₅₀ values of 0.11 and 0.10 μM for erythroid and myeloid progenitor proliferation (FIGS. 1A and 1C, respectively)—making ruxolitinib the most potent compound of the test compounds. Compound (1) inhibited erythroid progenitor proliferation at 10 and 3.33 μM and myeloid progenitor proliferation at 1.0 μM, which provided IC₅₀ values of 4.25 and 1.31 μM for erythroid and myeloid progenitor proliferation (FIGS. 1A and 1C, respectively)—making Compound (1) the least inhibitory on erythroid and myeloid progenitor cells.

A similar trend was seen in the megakaryocyte progenitor assay with momelotinib and pacritinib significantly inhibiting the total CFU-Mk from 10-0.333 μM, which provided IC₅₀ values of 0.30 and 0.34 μM, respectively (FIG. 1). Again, ruxolitinib was the most potent with significant inhibition of the total CFU-Mk at concentrations of 0.033 μM and higher, which provided IC₅₀ value of 0.2 μM (FIG. 1). Compound (1) inhibited the total CFU-Mk at 10, 3.33 and 1.0 μM, which provided IC₅₀ value of 1.56 μM—making Compound (1) the least inhibitory on megakaryocyte progenitor cells.

Of the compounds evaluated, Compound (1) had the least inhibition on progenitors of human erythroid (CFU-E, BFU-E), granulocyte-monocyte (CFU-GM), and multipotential (CFU-GEMM) lineages. The data indicates Compound (1) as a monotherapy is expected to be well tolerated with lower myelosuppressive adverse events compared to JAK inhibitors (e.g., ruxolitinib, pacritinib, and momelotinib). Accordingly, Compound (1) should be safe (A) to use in patients with normal or below normal platelets, red blood cells, and/or neutrophils and (B) at doses up to 3000 mg/day and in some embodiments more than 3000 mg/day.

### Example 2: In Vitro Hematological Tolerability of Compound (1), Ruxolitinib, Pacritinib, and Momelotinib in Combination

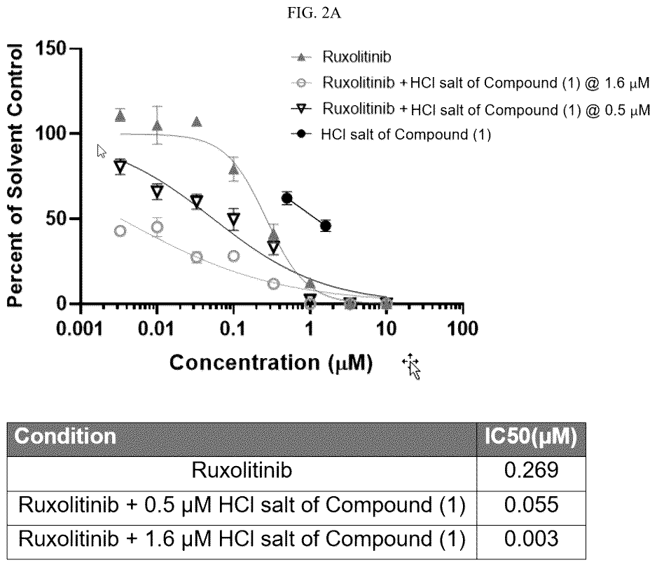

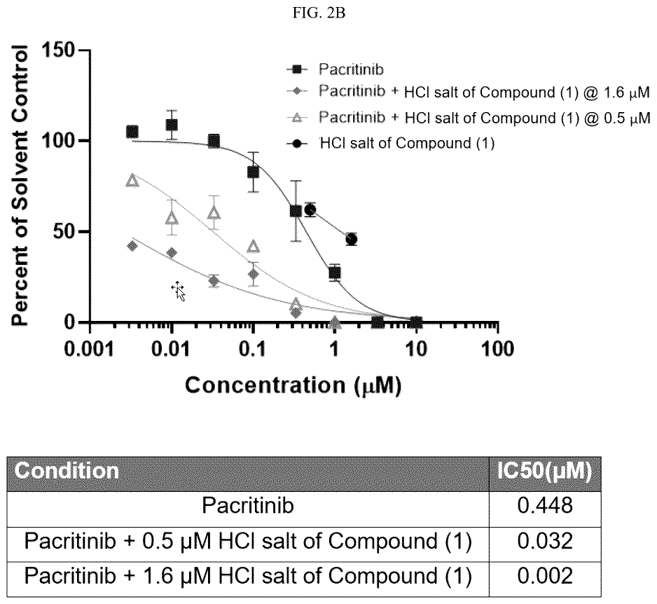

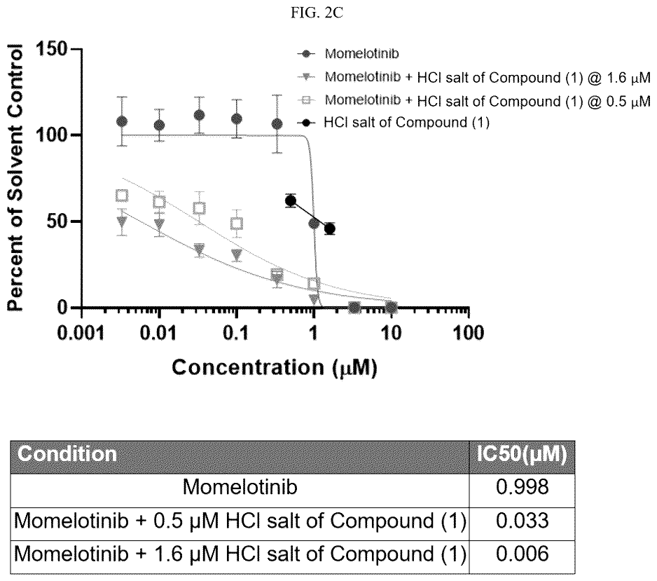

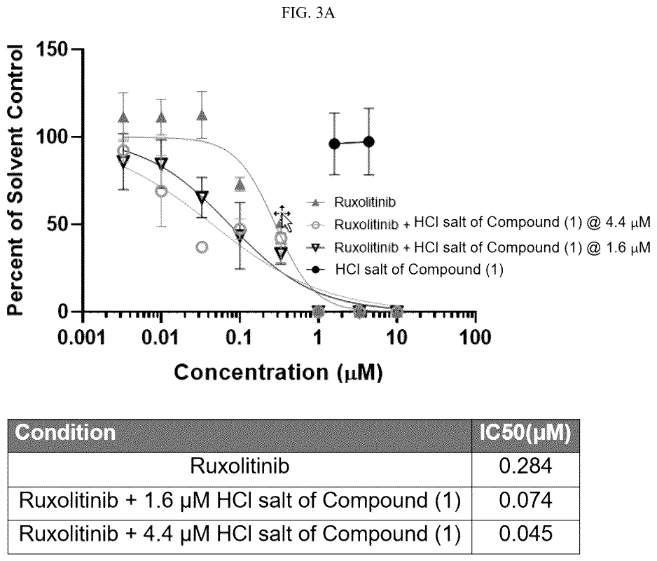

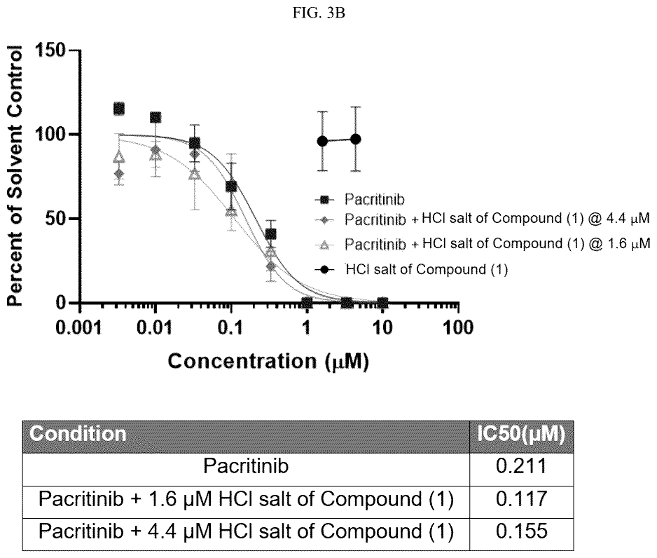

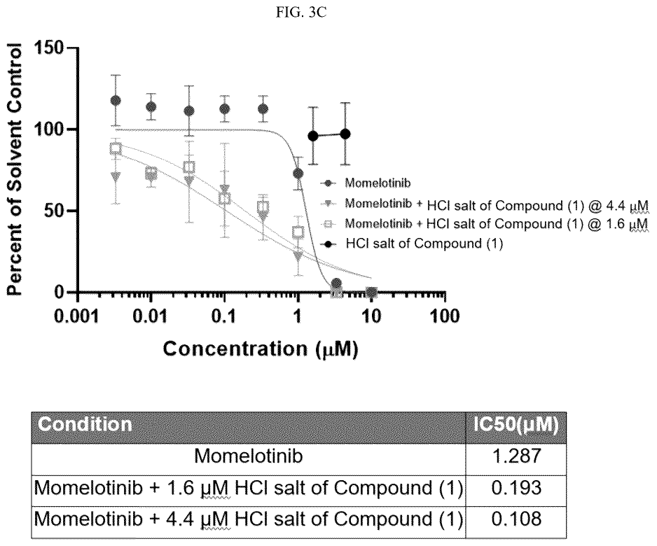

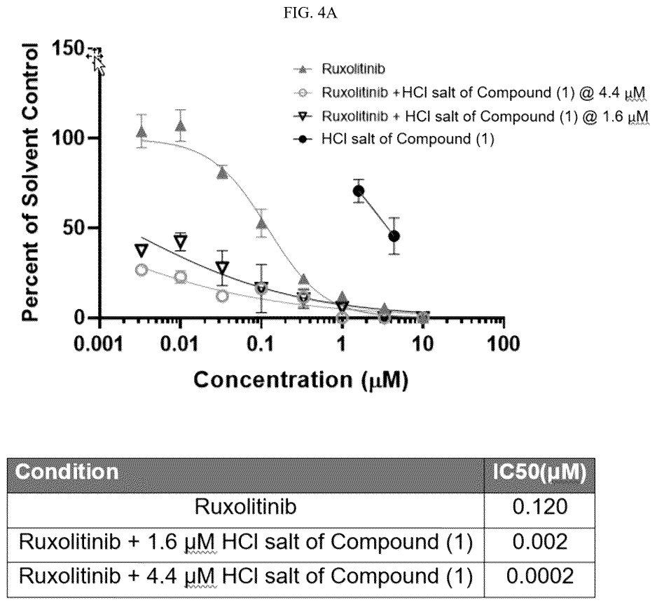

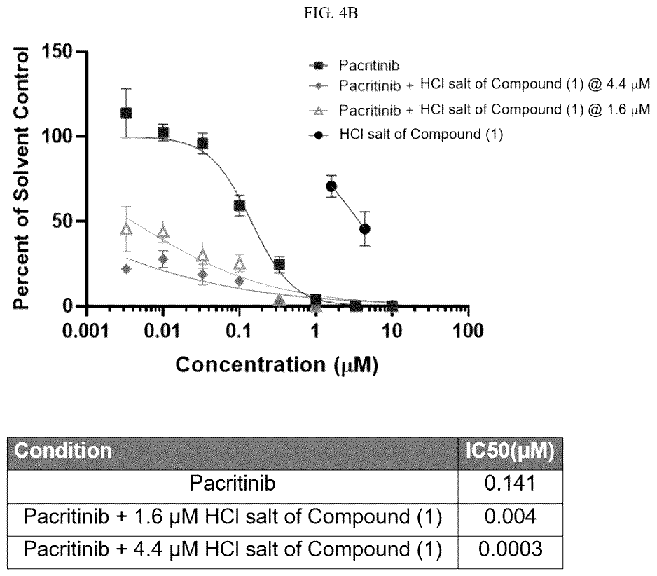

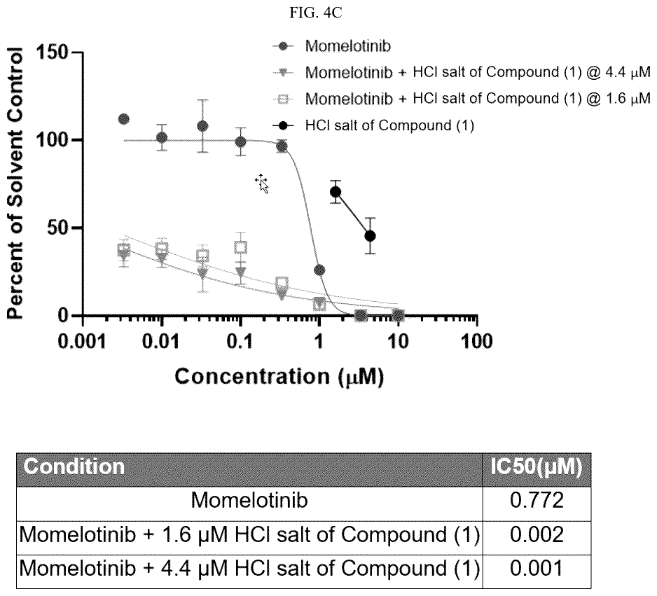

Following the method of Example 1, combinations of Compound (1) with ruxolitinib, pacritinib, or momelotinib were evaluated. The combinations were made by adding the HCl salt of Compound (1) to the standard dose responses of momelotinib, pacritinib and ruxolitinib to provide a concentration of the HCl salt of Compound (1) at 4.4 and 1.6 μM (human erythroid and myeloid progenitor proliferation) or 1.6 and 0.5 μM (human megakaryocyte progenitor proliferation). Other than the test compounds being combined, the only change from the method in Example 1 was a 0.2% DMSO solvent control instead of 0.1% DMSO. The results are provided in FIGS. 2A-4C (FIGS. 2A-2C are human megakaryocytes (CFU-MK), FIGS. 3A-3C are human erythroid (CFU-E, BFU-E), and FIGS. 4A-4C are human granulocyte-monocyte (CFU-GM).

The combination caused a greater change in the IC₅₀ value compared to the single agents of the myeloid progenitor (CFU-GM) (FIGS. 4A-4C) as compared to the erythroid progenitor (CFU-E, BFU-E) (FIGS. 3A-3C). The combination also caused more megakaryocyte inhibition (IC₅₀ values ranging from 0.002-0.055 μM) when compared to the single agents alone (FIGS. 2A-2C).

For all combinations, the addition of Compound (1) at 4.4, 1.6, and 0.5 μM to momelotinib, pacritinib, or ruxolitinib caused more inhibition of in vitro progenitor cells compared to the compounds alone (FIGS. 2A-4C). The increased in vitro inhibition suggests a possible increase of myelosuppressive adverse events when Compound (1) is used in combination (especially in combination with a JAK inhibitor) compared to use as a monotherapy. Accordingly, combination with Compound (1) should be safe (A) to use in patients with normal platelets, red blood cells, and/or neutrophils at doses at or below 3000 mg/day or (B) to use in patients with below normal platelets, red blood cells, and/or neutrophils at doses below 3000 mg/day (such as at or below 2000 mg/day).

### Example 3: Clinical Trial Study Treating MF Patients with HCl Salt of Compound (1)

Patients with MF (primary MF, post-PV MF, post-ET MF) and DIPSS intermediate-1, intermediate-2, or high risk were enrolled in a Phase 1/2 clinical study. All patients were previously treated with a JAK inhibitor and exhibited an inadequate response. Compound (1) was given to patients following a dose escalation to evaluate the safety and clinical response across different dose levels ranging from 480 mg QD to 720 mg BID. An interim analysis of doses administered to nine patients is reported in Table I.

TABLE I
Dose levels

| | Total | | |
|---|---|---|---|
| Dose | Daily | | Duration on monotherapy |
| Level | Dose | Number of | treatment with HCl salt of |
| (mg) | (mg) | Patients | Compound (1) |
| 480 QD | 480 | 1 completed | Up to 6 cycles |
| 720 QD | 720 | 1 completed, | At least 6 cycles |
| | | 1 active | |
| 360 BID | 720 | 1 completed | Up to 6 cycles |
| 480 BID | 960 | 2 completed, | At least 3 cycles |
| | | 2 actives | |
| 720 BID | 1440 | 1 active | At least 3 cycles |

QD = once a day;
BID = twice a day

Dose escalation to 960 mg BID and 1440 mg BID is intended.

Patients had to meet all of the following inclusion criteria to enroll in the study:
1. Adult (≥18 years of age)
2. Confirmed pathological diagnosis of primary MF (PMF) or post-PV-MF/postET-MF as per WHO diagnostic criteria and intermediate or high-risk primary or secondary MF based on the Dynamic International Prognostic Scoring System (DIPSS).
3. Previously treated with a JAK inhibitor and are intolerant, resistant, refractory or lost response to the JAK inhibitor, or are ineligible to be treated with ruxolitinib or fedratinib at the discretion of the investigator.
4. Grade≥2 bone marrow fibrosis, as confirmed by bone marrow biopsy within 12 weeks prior to Screening.
5. Fulfill the following laboratory parameters:
   a. Platelet count≥25×10⁹/L (without the assistance of growth factors or platelet transfusions)
   b. Absolute neutrophil count (ANC)≥1×10⁹/L without the assistance of granulocyte growth factors.
6. Peripheral blood blast count<10%.
7. Eastern Cooperative Oncology Group (ECOG) performance status≤2.
8. Life expectancy≥3 months.
9. Adequate renal function, as determined by clinical laboratory tests (serum creatinine≤1.5×upper limit of normal (ULN), and calculated creatinine clearance≥30 mL/min) (using Cockcroft-Gault formula).
10. Adequate hepatic function (ALT/AST≤3×ULN, total bilirubin≤1.5×ULN; or ALT/AST≤5×ULN, direct bilirubin≤2×ULN if due to MF); and coagulation ([PT and PTT]≤1.5×ULN).
11. Agree to provide bone marrow biopsies during the study: at baseline or within 12 weeks prior to enrollment, and every 6 months during treatment.
12. Capable of providing signed informed consent as described in which includes compliance with the requirements and restrictions listed in the informed consent form (ICF) and in this protocol
13. Non-fertile or agree to use an adequate method of contraception as described while on study and for 6 months following the study, and have a negative pregnancy test (if female of childbearing potential and not currently nursing; males agree to use an adequate method of contraception as described in while on study and for 3 months following the study. Note: Female breastfeeding patients may be enrolled if they interrupt breastfeeding. Breastfeeding should not be resumed for at least 6 months after the last dose of study drug.
14. Splenomegaly during the screening period as demonstrated by splenic length≥5 cm below the costal margin by palpation or spleen volume of ≥450 cm³ by magnetic resonance Imaging (MRI) or computerized tomography (CT) scan
15. Show at least 2 symptoms measurable (score≥1) using the Myelofibrosis Symptom Assessment Form (MFSAF), v4.0.
16. Able to take orally administered medication

Patients meeting any one of the exclusion criteria were prohibited from participating in the study:
1. Received previous systemic antineoplastic therapy (including unconjugated therapeutic antibodies, toxin immunoconjugates, ESA, and alpha-interferon) or any experimental therapy within 14 days or 5 half-lives, whichever is longer, before the first dose of study treatment.
2. Major surgery within 2 weeks before the first dose of either study drug.
3. Splenic irradiation within 6 months prior to Screening or prior splenectomy.
4. AML, MDS, or peripheral blasts≥10%.
5. Prior autologous or allogeneic stem cell transplant at any time.
6. Eligible for allogeneic bone marrow or stem cell transplantation within 3 months following enrollment (not a candidate or not willing to undergo transplantation or a suitable donor not available are considered as transplant ineligible).
7. Currently receiving treatment with a prohibited medication that cannot be discontinued at least one week prior to the start of treatment.
8. Experiencing electrolyte abnormalities of NCI CTCAE Grade≥2 (e.g., serum potassium, magnesium and calcium) unless they can be corrected during screening and are deemed not clinically significant by the Investigator.
9. History of congestive heart failure, myocardial infarction within the past 6 months prior to Cycle 1/Day 1; left ventricular ejection fraction<45% by echocardiogram or MUGA, unstable arrhythmia, or evidence of ischemia on electrocardiogram (ECG) within 14 days prior to Cycle 1/Day 1.
10. Corrected QT interval (using Fridericia's correction formula) of >450 msec in men and >470 msec in women.
11. Central nervous system (CNS) cancer or metastases, meningeal carcinomatosis, malignant seizures, or a disease that either causes or threatens neurologic compromise (e.g., unstable vertebral metastases).
12. Other invasive malignancies within the last 3 years, except non-melanoma skin cancer, and localized cured prostate and cervical cancer
13. Experienced portal hypertension or any of its complications.
14. Active, uncontrolled bacterial, viral, or fungal infections, requiring systemic antimicrobial within 14 days.
15. Known bleeding diathesis or signs of uncontrolled active bleeding (hematuria, GI bleeding) other than self-limited causes of benign etiology that have been adequately investigated at the discretion of the Investigator.
16. Requiring anticoagulation with aspirin>81 mg daily, unfractionated heparin, low molecular weight heparin (LMWH), direct anti-thrombin inhibitors, or vitamin K antagonists (e.g., warfarin).
17. Severe chronic obstructive pulmonary disease with hypoxemia (defined as resting 02 saturation of <90% breathing room air).
18. Unwilling or unable to comply with procedures required in this protocol
19. Known infection with human immunodeficiency virus, hepatitis B, or hepatitis C. Patients with history of chronic hepatitis that is currently not active are eligible.
20. Serious nonmalignant disease (e.g., hydronephrosis, liver failure, or other conditions) that could compromise protocol objectives in the opinion of the Investigator and/or the Sponsor.
21. Currently receiving any other investigational agent.
22. Exhibited allergic reactions to a similar structural compound, biological agent, or formulation.
23. Medical condition or have undergone significant surgery to the gastrointestinal tract that could impair absorption or that could result in short bowel syndrome with diarrhea due to malabsorption.
24. Used hydroxyurea or anagrelide within 24 hours prior to the first dose.
25. Systemic steroid therapy (>10 mg daily prednisone or equivalent) within 7 days prior to the first dose ofstudy treatment (note: topical, inhaled, nasal, and ophthalmic steroids are not prohibited).

The initial trial design required patients to have platelet counts≥50×10⁹/L and Hgb levels≥8 but the Hgb requirement was subsequently removed, and the platelet count requirement reduced to 25×10⁹/L, in response to positive hematological responses in the patients initially enrolled.

The following procedures and tests in Table II were performed for all enrolled patients:

TABLE II

| Procedures/Tests | Frequency |
|---|---|
| Record Demographic information | Once before treatment |
| Record Medical history | Once before treatment |
| Record Transfusion History/Status | Once before treatment |
| Physical examination | Once every cycle (28 days) |
| Vital signs | Once every cycle (28 days) |
| ECOG performance status | Once every cycle (28 days) |
| Hematology | Once every cycle (28 days) |
| Chemistry | Once every cycle (28 days) |
| Coagulation | Once every cycle (28 days) |
| Urinalysis | Once every cycle (28 days) |
| Pregnancy test | Once every cycle (28 days) |
| Serology for Hep B, Hep C | Once every cycle (28 days) |
| Record Prior and concomitant medication | Every visit |
| Spleen by Palpation | Once every cycle (28 days) |
| Spleen Volume by Imaging (MRI or CT scan) | Once before treatment, then every 12 weeks |
| IWG-MRT assessment | Every 12 weeks |
| MFSAF (v4.0) Diary | Patient completes daily from 7 days prior to Cycle |
| | 1, Day 1 through the last day of treatment |
| PGIC Diary | Once every cycle (28 days) |
| Bone marrow biopsy | Every 24 weeks |
| PK blood sample | C1D1, C1D8, C1D15, C1D22, C2D1 |
| 12-lead ECG | Once every cycle (28 days) |
| Holter ECG Monitor | C1D1 and C2D1 |
| ECHO/MUGA | Once before treatment |
| AEs assessment | Every visit |
| Biomarker blood samples | Once every cycle (28 days) |
| Survival Follow-up | For 18 months after treatment |

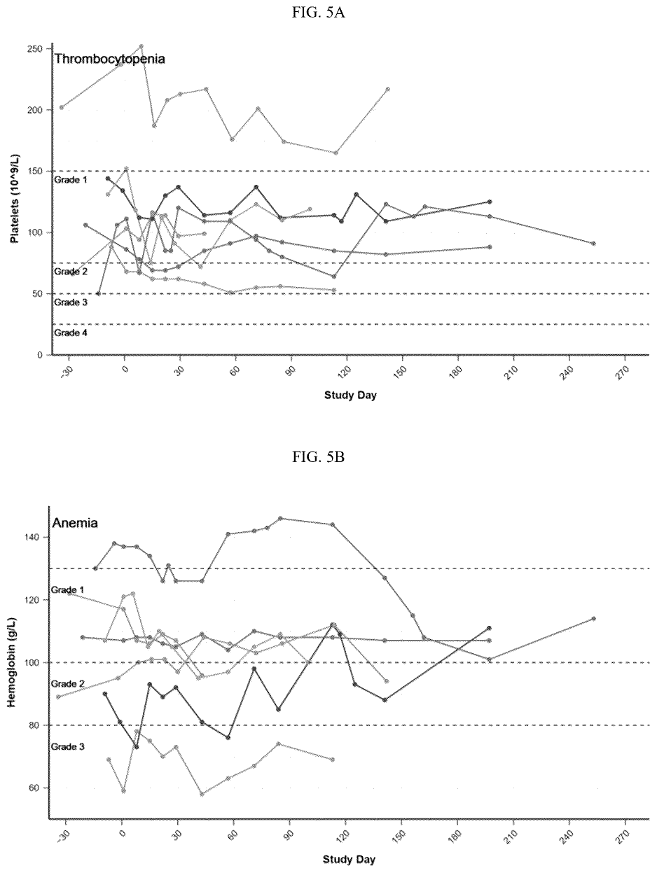

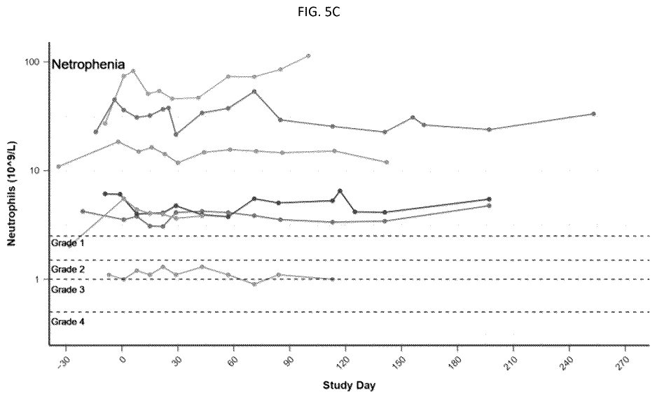

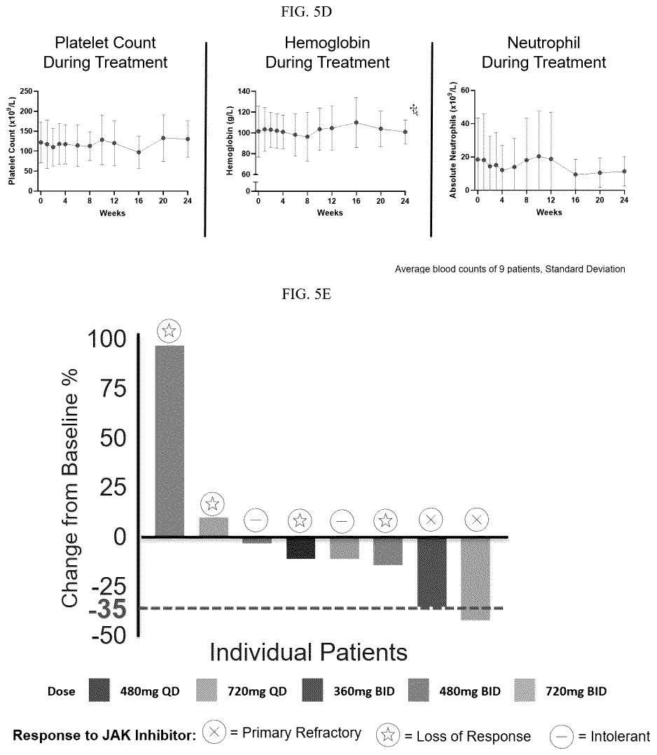

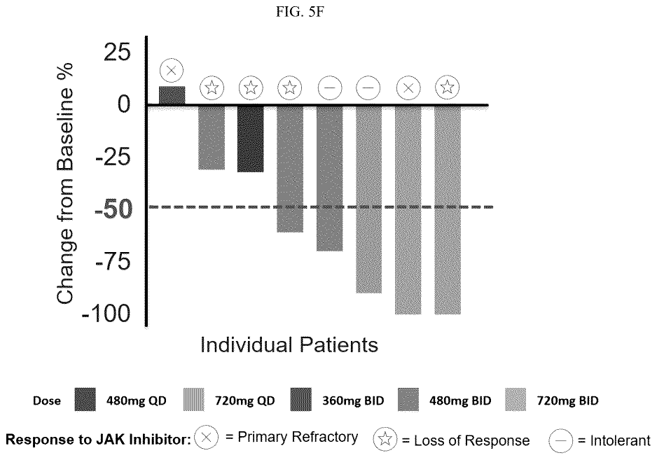

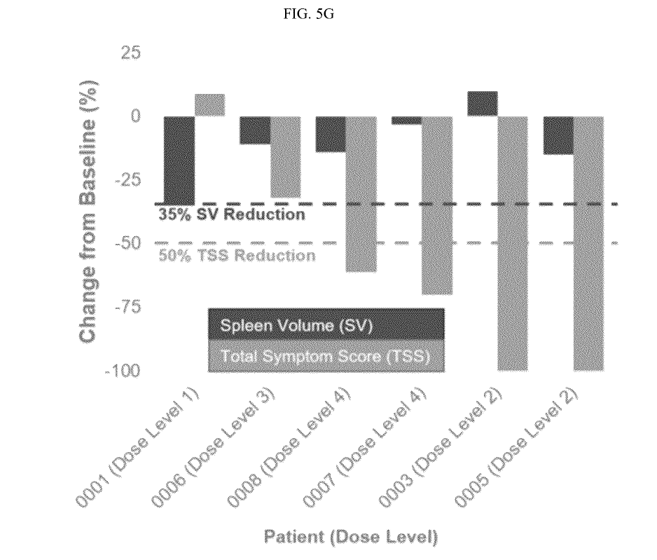

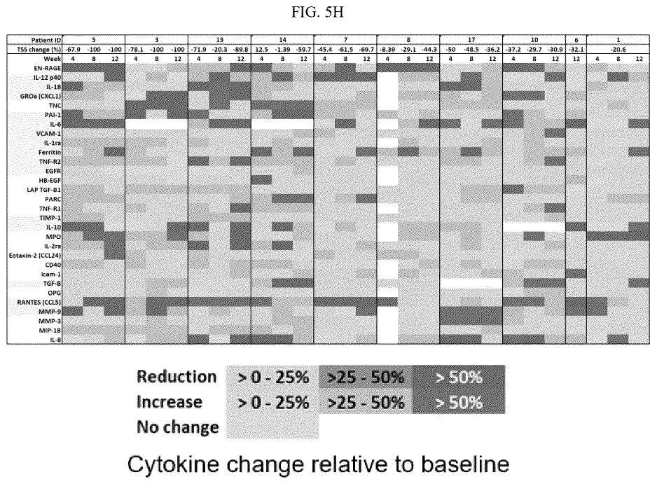

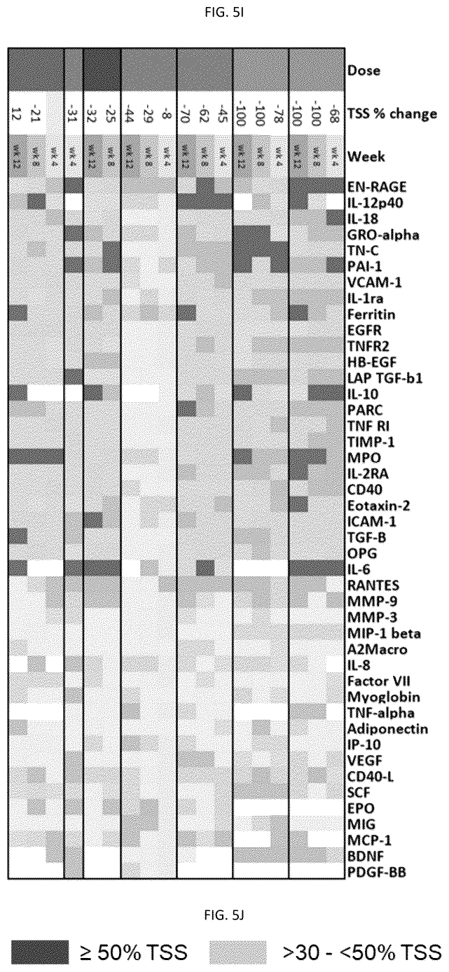

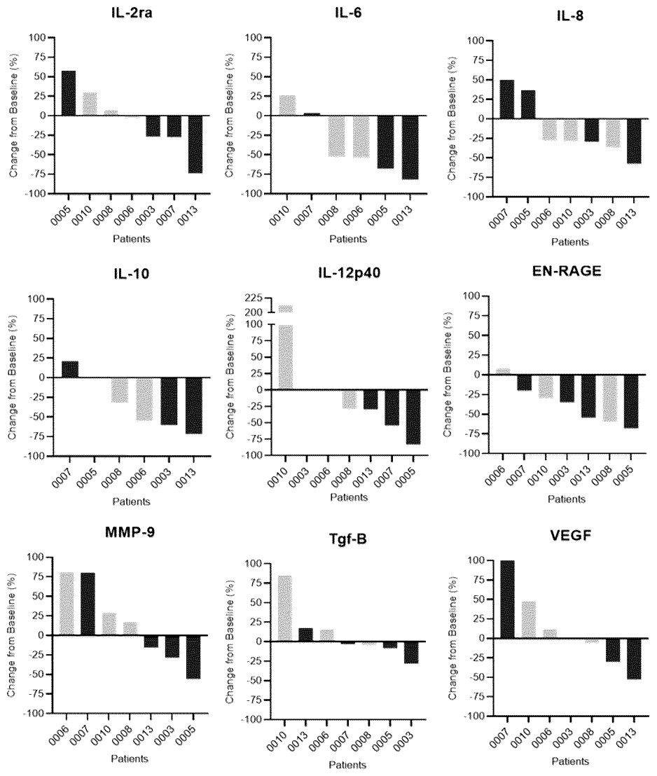

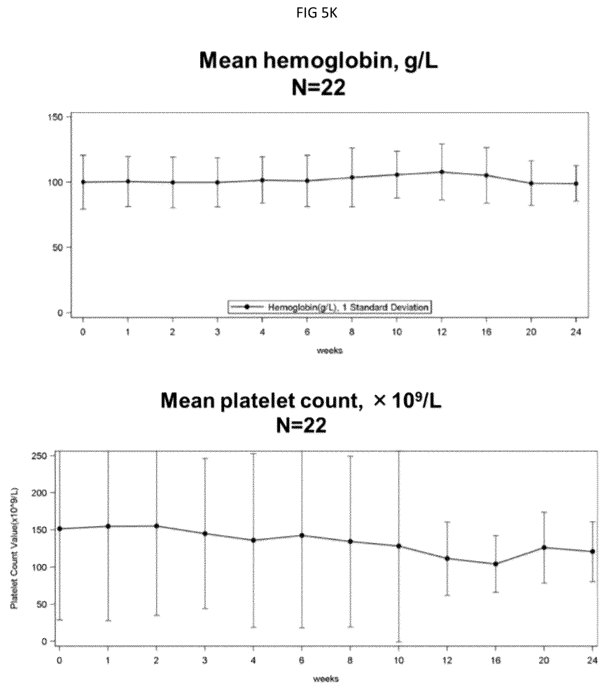

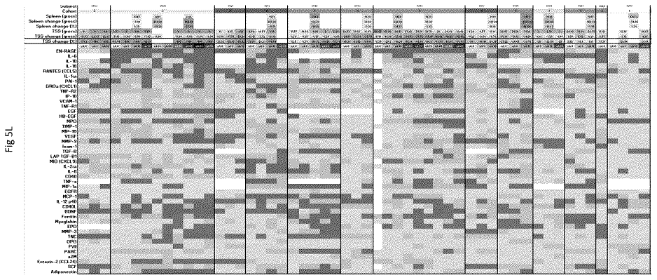

In patients treated with the HCl salt of Compound (1), platelet counts were stable or increased (FIG. 5A), hemoglobin increased in some patients (FIG. 5B), and the neutrophil counts were stable (FIG. 5C). The average blood count values for the patients during treatment are provided in FIG. 5D. Eight out of 10 patients with baseline measurements (i.e., spleen volume was measured before treatment began) exhibited spleen volume reduction over their treatment duration (≥12 weeks) with the HCl salt of Compound (1) at the doses provided in Table 1. The best spleen volume reduction ranged from 3 to 42% (median best change −13%) (FIG. 5E) over their treatment duration. Nine out of 10 patients with baseline measurements (i.e., total symptom score was measured before treatment began) exhibited total symptom reduction over their treatment duration (≥12 weeks) with the HCl salt of Compound (1) at the doses provided in Table 1. The best total symptom reduction ranged from 31 to 100% (median best change −70%) (FIG. 5F). As shown in FIG. 5G, 4 patients exhibited reduced spleen size and reduced symptoms, while 1 patient exhibited spleen volume reduction and increased symptoms and 1 patient exhibited spleen volume increase and reduced symptoms. Cytokine modulation was observed as early as within the first 24 hours. Cytokine changes over treatment duration was available for 7 patients. More reductions in cytokines were observed in patients who had more total symptoms reduction (FIGS. 5H, 5I, and 5J). Bone marrow fibrosis reduction from grade 3 to 2 was seen in one patient who also achieved spleen and symptoms responses and showed reductions in MF associated cytokines: 1L6 (68%), IL12p40 (83%), MMP9 (56%), and EN-RAGE (68%), and was on active treatment for >18 months. Myelosuppressive adverse events (i.e., anemia, neutropenia, thrombocytopenia) were observed in 1 patient.

Compound (1) monotherapy showed spleen volume reduction and total symptoms reduction in patients previously treated with JAK inhibitors. For most patients, less than 1 grade change was observed for platelet count, hemoglobin, and neutrophil reduction (i.e., myelosuppressive adverse events). The results of this study indicate that Compound (1) can be safely used (A) in patients with normal or below normal platelets, hemoglobin, and/or neutrophils and (B) at doses up to 3000 mg/day and in some embodiments more than 3000 mg/day. Not wishing to be bound by theory, this study indicates that such treatment modality will be expected to provide a reduction in inflammatory cytokines (including those associated with MF symptoms) and a reduction in bone marrow fibrosis will be observed. It is expected that reduction in inflammatory cytokines (including those associated with MF symptoms) may be reduced, as well as a decrease in bone marrow fibrosis.

A second interim analysis of baseline patient characteristics after enrolling 23 patients in the study is reported in Table III:

TABLE III

| | |
|---|---|
| Parameter | Results |

Patient Characteristics

| | |
|---|---|
| Age, median (range) | 73 years (61-80) |
| Spleen volume, median (range) | 2008.2 cm³ (609-6006) |
| Total Symptoms Score (MF-SAF v4.0), median (range) | 20.8 (4-62) |
| Platelet count, median (range) | 115 × 10⁹/L (64-520) |
| Hemoglobin, median (range) | 10.1 g/dL (5.9-13.7) |
| Transfusion dependence (n, %) | 5 (33) |

Disease Characteristics

| | |
|---|---|
| MF subtypes (n, %) | |
| Primary | 11 (47.8) |
| Post-polycythemia vera | 10 (43.5) |
| Post-essential thrombocythemia | 2 (8.7) |
| DIPSS Risk Group (n, %) | |
| Intermediate-1 | 7 (30.4) |
| Intermediate-2 | 12 (52.2) |
| High | 4 (17.4) |
| Driver mutations (n, %) | |
| JAK2V617F | 18 (78) |
| CALR | 4 (17) |
| MPL | 2 (9) |
| Duration of prior JAK inhibitor, median (range) | 12.9 months (2.3-81.0) |
| Response to JAK inhibitors (n, %) | |
| Primary refractory | 5 (22) |
| Loss of response | 11 (48) |
| Intolerant | 6 (26) |

DIPSS = Dynamic International Prognostic Scoring System;
JAK = Janus kinase;
MF = myelofibrosis;
MF-SAF = myelofibrosis symptom assessment form;
n = number

Altogether, 23 patients were enrolled at this interim timepoint across the 5 dose levels reported in Table 1, with no dose limiting toxicities reported and doses up to 720 mg BTD declared safe. Mean hemoglobin and platelet counts throughout 24-weeks of treatment for 22-evaluable patients are depicted in FIG. 5K. An updated heat map depicting cytokine reductions substantially at this interim timepoint is provided in FIG. 5L.

As of the interim analysis, 13 patients had completed≥12 weeks of treatment and were evaluable for spleen volume change assessment. Of the 13 patients, 10 experienced spleen volume reduction (SVR) during treatment. Median spleen volume change was −11% and 3 of 13 (23%) patients achieved≥35% SVR.

In addition to the splenic response, there was a clear reduction in the symptom burden. Symptoms were assessed using the Myelofibrosis Symptom Assessment Form v4.0. Overall, 12 out of 13 patients have had TSS reduction, with a median reduction of 70% from baseline. Seven of 13 (54%) patients had ≥50% TSS reduction, and 5 of these patients demonstrated durable response for ≥12 weeks.

Table IV compares the results reported at this interim analysis, in relapsed/refractory patients, to two approved JAK therapies, and three other treatments under development or previously under development for the treatment of myelofibrosis:

TABLE IV

| | | | | | SVR35 at | TSS50 at |
|---|---|---|---|---|---|---|
| Drug | MOA | Study Population | | Regimen | Week 24 | Week 24 |
| Pacritinib | JAKi | R/R MF | <50 × 10⁹/L | Monotherapy | 29% | Endpoint not |
| | | | PLT | | | met |
| Momelotinib | JAKi | R/R MF | ≥25 × 10⁹/L | Monotherapy | 23% | 25% |
| | ACVR1i | | PLT | | | |
| Navitoclax | BCL-2, | R/R MF | ≥100 × 10⁹/L | Monotherapy | 7% | 10% |
| | BCL-XL | | PLT | | | |
| | Inhibitor | | | | | |
| Parsaclisib | P13K | R/R MF | ≥50 × 10⁹/L | RUX+Paraclisib | 3% | 11% |
| | Inhibitor | | PLT | | | |
| Pelabresib | BET Inhibitor | R/R MF | ≥75 × 10⁹/L | Monotherapy | 11% | 28% |
| | | | PLT | | | |
| TP-3654 | PIM1 | R/R MF | ≥25 × 10⁹/L | Monotherapy | 23% | 54% |
| | Inhibitor | | PLT | | | |

Toxicities were also evaluated from this interim dataset and tabulated in Table V, relative to toxicities reported for other MF treatments. As can be seen, there is minimal or no indication of overlapping hematological toxicities (thrombocytopenia and/or anemia) making Compound (1) an ideal partner with other MF treatments including ruxolitinib and momelotinib.

TABLE V

| | | | Momelotinib | | Ruxolitinib | | Fedratinib | | Pacritinib | |
|---|---|---|---|---|---|---|---|---|---|---|
| | TP-3654-102 | | MOMENTUM | | COMFORT 1 | | JAKARTA | | PERSIST 2 | |
| | Indication | | | | | | | | | |
| | | | R/R MF | | | | | | | |
| | | | PLT ≥25, | | Tx Naïve | | Tx Naïve | | Tx Naïve | |
| | R/R MF | | Hgb <10 g/dl | | PLT ≥100 | | PLT ≥50 | | PLT ≤100 | |
| | Treatment | | | | | | | | | |
| | | | Monotherapy | | Monotherapy (5- | | Monotherapy | | Monotherapy | |
| | Monotherapy | | (200 mg QD) | | 25 mg BID) | | (400 mg QD) | | (200 mg BID) | |
| | Phase/# of Subjects | | | | | | | | | |
| | Phase 1/2; N = 23 | | Phase 3; N = 195 | | Phase 3; N = 155 | | Phase 3; N = 96 | | Phase 3; N = 106 | |
| | TEAE/Grade | | | | | | | | | |
| | All Gr | ≥3 Gr | All Gr | ≥3 Gr | All Gr | ≥3 Gr | All Gr | ≥3 Gr | All Gr | ≥3 Gr |
| | (%) | (%) | (%) | (%) | (%) | (%) | (%) | (%) | (%) | (%) |
| Thrombocytopenia | 22 | 13 | 76 | 28 | 70 | 13 | 63 | 17 | 34 | 32 |
| Anemia | 13 | 4.3 | 99 | 61 | 96 | 45 | 99 | 43 | 24 | 22 |
| Neutropenia | 0 | 0 | 29 | 12 | 19 | 7 | 28 | 8 | — | 7 |

The MF clinical data reported in this disclosure for Compound (1), including the efficacy data, the myelosuppressive data, the fibrosis data, and the spleen size reduction data, can reasonably be expected to occur in other myeloproliferative neoplasms including, but not limited to, polycythemia vera (PV), primary or essential thrombocythemia (ET), pre-MF, primary or idiopathic MF, and secondary MF (e.g., MF secondary to polycythemia vera or essential thrombocythemia). This expectation is derived from, among other facts, increased PIM1 expression is observed in ET and PV (Rampal R, Al-Shahrour F, Abdel-Wahab O, Patel J P et al. Integrated genomic analysis illustrates the central role of JAK-STAT pathway activation in myeloproliferative neoplasm pathogenesis. Blood 2014 May 29; 123(22)), as well as the fact that JAK2 and CALR activating mutations are also observed in PV and ET (Greenfield G, McPherson S, Mills K, McMullin M F, The ruxolitinib effect: understanding how molecular pathogenesis and epigenetic dysregulation impact therapeutic efficacy in myeloproliferative neoplasms. Journal of translational medicine 2018 December; Vol. 16, p. 360), and the mouse models of PV and ET reported in the literature (e.g. Li J, Kent D G, Chen E, and Green A R, Mouse models of myeloproliferative neoplasms: JAK of all grades. Dis Model Mech. 2011 May; 4(3): 311-317) essentially are modified versions of the work using Compound (1) in mouse models reported in the literature (e.g. Nath D, Dutta A, Yang Y, Whatcott C, et al., The PIM Kinase Inhibitor TP-3654 in Combination with Ruxolitinib Exhibits Marked Improvement of Myelofibrosis in Murine Models. Blood (2018) 132 (Suppl_1): 54) (single copy of JAK2V617F mutation instead of double).

### Example 4: Clinical Trial Study Treating MF Patients with a Combination of Compound (1) and Ruxolitinib

For combination treatment with the HCl salt of Compound (1), patient enrollment will be substantially the same as described in Example 3. However, patients will be on a stable dose of ruxolitinib from 5 mg QD to 25 mg BID (no dose change within the previous 12 weeks) for treating MF with a platelet count of ≥50×10⁹/L and exhibiting an inadequate response (e.g., an inferior response and/or relapse and/or refractory MF disease). The stable dosing of ruxolitinib will continue and Compound (1) in dose from 240 or 360 mg BID to 1440 mg BID will be added to these patient's treatment regimen. Safety and clinical response will be evaluated.

We expect to observe spleen volume reduction and/or total symptoms improvement. We do not expect patients to exhibit adverse effects to platelet count, neutrophil count, or red blood cells. Rather, we expect patients receiving the combination therapy will exhibit a normalization or improvement in platelet count and/or neutrophil count and/or hemoglobin, and/or transfusion improvement including conversion into transfusion independence. We also expect patients to exhibit a reduction or normalization of cytokines and/or improvement in bone marrow fibrosis by at least 1 grade. Overall, the combination therapy of the HCl salt of Compound (1) with ruxolitinib should lead to an increase in survival and/or decreased rate of leukemia transformation and/or transplant eligibility for patients having an acceptable safety profile.

In carrying out this protocol, one or more of the following will be observed in some or all of the patient population: spleen volume reduction; improvement in total symptom scores. Based upon the foregoing and without wanting to be bound by theory, it is not expected that any of the following will be observed in the patient population: (i) adverse effects to platelet count; (ii) adverse effects to neutrophil count; or (iii) adverse effects to hemoglobin count. Accordingly, it will be observed that patients receiving the combination therapy will exhibit a normalization or improvement in platelet count, neutrophil count, hemoglobin, and/or transfusion improvement, which may include conversion into transfusion independence. It will also be observed that patients will exhibit a reduction or normalization of cytokines and/or improvement in bone marrow fibrosis by at least 1 grade. Overall, the combination therapy of the HCl salt of Compound (1) with ruxolitinib will be seen to provide an increase in survival and/or decreased rate of leukemia transformation and/or a decrease in patients becoming transplant eligible with an acceptable safety profile.

### Example 5: Clinical Trial Study Treating MF Patients with a Combination of Compound (1) and Momelotinib

In combination treatment with the HCl salt of Compound (1), patient enrollment will be substantially the same as described in Example 3, however, patients will be (A) newly diagnosed with MF or (B) have an inadequate response to a previous treatment with a JAK inhibitor (e.g. ruxolitinib or fedratinib), and, at least at the beginning of the study, will have a platelet count≥50×10⁹/L. Patients will receive momelotinib from 100 to 200 mg QD and Compound (1) in dose from 240 mg QD to 1440 mg BID. Safety and clinical response will be evaluated.

It is expected in this treatment population, one or more of the following will be observed in some or all of the patient population: spleen volume reduction; improvement in total symptom scores. Based upon the foregoing and without wanting to be bound by theory, it is not expected that any of the following will be observed in the patient population: (i) adverse effects to platelet count; (ii) adverse effects to neutrophil count; or (iii) adverse effects to hemoglobin count. Accordingly, it will be observed that patients receiving the combination therapy will exhibit a normalization or improvement in platelet count, neutrophil count, hemoglobin, and/or transfusion improvement, which may include conversion into transfusion independence. It will also be observed that patients will exhibit a reduction or normalization of cytokines and/or improvement in bone marrow fibrosis by at least 1 grade. Overall, the combination therapy of the HCl salt of Compound (1) with momelotinib is expected to lead to an increase in survival and/or decreased rate of leukemia transformation and/or a decrease in patients becoming transplant eligible with an acceptable safety profile.

### Example 6: Clinical Trial Study Treating MF Patients with a Combination of Compound (1) and Pacritinib

For combination treatment with the HCl salt of Compound (1), patient enrollment will be substantially the same as described in Example 3. However, patients will be (A) newly diagnosed with MF, (B) have relapsed and/or refractory to a previous treatment with a JAK inhibitor (e.g. ruxolitinib or fedratinib), or (C) discontinued from JAK inhibitor therapy due to adverse event. Patients will receive pacritinib from 100 to 200 mg QD and Compound (1) in dose from 240 mg QD to 1440 mg BID. Safety and clinical response will be evaluated.

It is expected in this treatment population, one or more of the following will be observed in some or all of the patient population: spleen volume reduction; improvement in total symptom scores. Based upon the foregoing and without wanting to be bound by theory, it is not expected that any of the following will be observed in the patient population: (i) adverse effects to platelet count; (ii) adverse effects to neutrophil count; or (iii) adverse effects to hemoglobin count. Accordingly, it will be observed that patients receiving the combination therapy will exhibit a normalization or improvement in platelet count, neutrophil count, hemoglobin, and/or transfusion improvement, which may include conversion into transfusion independence. It will also be observed that patients will exhibit a reduction or normalization of cytokines and/or improvement in bone marrow fibrosis by at least 1 grade. Overall, the combination therapy of the HCl salt of Compound (1) with pacritinib is expected to lead to an increase in survival and/or decreased rate of leukemia transformation and/or a decrease in patients becoming transplant eligible with an acceptable safety profile.

### Example 7: Hematological Evaluation of Selective PIM1 Kinase Inhibitors and their Impact on Platelet Counts

In order to determine the impact off PIM1 selectivity on hematological function, a comparative analysis was undertaken comparing human clinical data reported for 5 different PIM kinase inhibitors. Results are reported in the following Table VI:

TABLE VI

| | | | | CLINICAL | | | |
|---|---|---|---|---|---|---|---|
| Kinase | Preclinical IC50 (nM) | | | Indication | | Non-hem AE | |
| Inhibitor | PIM1 | PIM2 | PIM3 | (Ph 1) | Efficacy | (>10%) | Hem AE (>10%) |
| Cmpd 1 | 5 | 239 | 42 | R/R MF | | G1/2 N/V/D | No ≥G3 |
| | | | | | | | thrombocytopenia |
| PIM447 | 0.006 | 0.018 | 0.009 | R/R MM | ORR 8.9% | G3+ fatigue (8.9%), | G3+ thrombocytopenia |
| | | | | (n = 79) | DCR 72.2% | G3+ diarrhea | (33%) |
| | | | | | PFS 10.9 months | (6.3%), | G3+ anemia (20%), |
| | | | | | | pneumonia (7.6%) | G3+ neutropenia |
| | | | | | | | (20%) |
| INCB053914 | 0.24 | 30 | 0.12 | R/R AML | Not reported | ALT/AST increased | G3+ Anemia (18%) |
| | | | | (n = 30); | | (36%), | G3+ |
| | | | | Other (n = 9) | | nausea (23%), | Thrombocytopenia |
| | | | | | | diarrhea (21%), rash | (13%) |
| | | | | | | (15%) | |
| AZD1208 | 0.4 | 5 | 1.9 | R/R AML | No clinical | Nausea (47%) | Febrile neutropenia |
| | | | | (n = 32) | response, reduction | diarrhea (44%), | (28%) |
| | | | | | in circulating blasts | vomiting (31%), | |
| | | | | | in several patients | pneumonia (19%) | |
| | | | | Solid tumor | Stable disease for | Diarrhea (83%), | Anemia (51%), |
| | | | | (n = 35) | ≥6 weeks in 13 | nausea (74%) | Thrombocytopenia |
| | | | | | patients | vomiting (54%), | (14-26%) |
| | | | | | | | WBC decreased (26%) |

Further confirmation of the platelet sparing effects of selective PIM1 kinase inhibitors can be obtained from a CGLP preclinical dog study with SGI-1776, which has PIM1/PIM2/PIM3 IC₅₀ values of 12, 980, and 20, respectively. Hematology parameters in either sex were not considered to be altered by SGI-1776 at 10 mg/kg/day. The only hematology parameter altered at 30 mg/kg/day were the platelet counts (PLT) which were increased 16.5% in males and 12.1% in females. At 90 mg/kg/day, PLT increases were 20% in males and 26.3% in females.

Based on the foregoing analysis, it can reasonably be expected that TP-3654 as compared to pan-PIM inhibitors spares megakaryocytopoiesis (cell numbers and ploidy) and affects cytokine elaboration by megakaryocytes (MK) generated in vitro from mononuclear cells (MNC) form the peripheral blood or bone marrow aspirates from PV, ET, pre-MF and MF patients and normal controls. Improvements versus pan-PIM inhibitors can further be expected on (1) viability, (2) cell number and (3) apoptosis of MNCs isolated from peripheral blood or bone marrow aspirates of PV, ET, pre-MF and MF patients with JAK2V617F and normal controls before (0 hrs, baseline) and after treatment with TP-3654 for 24 hrs, 48 hrs and 72 hrs, and on MPN driver mutated hematopoietic colony formation after 48 hrs of treatment.

All of the U.S. patents, U.S. patent application publications, U.S. patent applications, foreign patents, foreign patent applications and non-patent publications referred to in this specification are incorporated herein by reference, in their entirety to the extent not inconsistent with the present description.

From the foregoing it will be appreciated that, although specific embodiments of the disclosure have been described herein for purposes of illustration, various modifications may be made without deviating from the spirit and scope of the disclosure.

1. A method for treating a myeloproliferative neoplasm in a human subject in need thereof, comprising administering to the subject a therapeutically effective amount of compound (1): 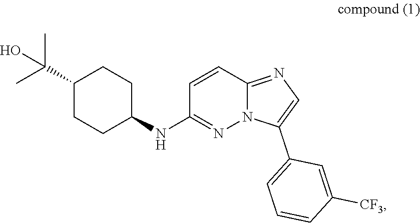 [SMILES: CC(C)(O)[C@H]1CC[C@H](Nc2ccc3ncc(-c4cccc(C(F)(F)F)c4)n3n2)CC1]
or a pharmaceutically acceptable salt thereof,
wherein the subject has poor bone marrow functioning characterized by:
(i) thrombocytopenia;
(ii) anemia;
(iii) transfusion dependence; or
(iv) a combination thereof;
and wherein the treatment induces a clinically meaningful response in the subject.

2-3. (canceled)

4. A method of treating a myeloproliferative neoplasm by inducing:
(a) TSS50 in a human subject in need thereof;
(b) a probability of SVR20 in a human subject affected by the myeloproliferative neoplasm about ≥15%; or
(c) a combination of (a) and (b),
the method comprising administering to the subject a therapeutically effective amount of compound (1): 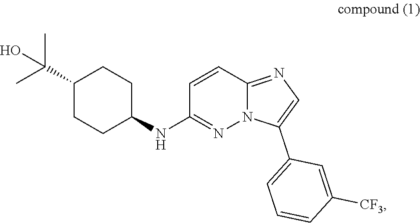 [SMILES: CC(C)(O)[C@H]1CC[C@H](Nc2ccc3ncc(-c4cccc(C(F)(F)F)c4)n3n2)CC1]
or a pharmaceutically acceptable salt thereof,
wherein the myeloproliferative neoplasm is treated by inducing TSS50 in the subject, the treatment induces a probability of SVR20 in the subject about ≥15%, or a combination thereof.

5-6. (canceled)

7. The method of claim 4, wherein the subject has previously been treated by a JAK inhibitor for the myeloproliferative neoplasm and optionally has relapsed disease or is refractory to the JAK inhibitor.

8. (canceled)

9. The method of claim 4, wherein the subject has poor bone marrow functioning characterized by:
(i) thrombocytopenia;
(ii) anemia;
(iii) transfusion dependence; or
(iv) a combination thereof.

10. The method of claim 1, wherein the subject has previously been treated by a JAK inhibitor for the myeloproliferative neoplasm and optionally has relapsed disease or is refractory to the JAK inhibitor.

11-15. (canceled)

16. The method of claim 1, comprising administering to the subject the compound (1) or the pharmaceutically acceptable salt thereof and ruxolitinib or a pharmaceutically acceptable salt thereof or momelotinib or a pharmaceutically acceptable salt thereof in amounts that induce no overlapping hematological toxicities in terms of anemia and thrombocytopenia and transfusion dependence.

17. (canceled)

18. The method of claim 1, by inducing SVR35 and TSS50 in the subject, wherein the myeloproliferative neoplasm is treated by inducing SVR35 and TSS50 in the subject.

19-24. (canceled)

25. The method of claim 1, wherein the clinically meaningful response is selected from:
(a) complete remission;
(b) partial remission;
(c) clinical improvement; and
(d) stable disease;
wherein the clinically meaningful response is evaluated by IWG-MRT response criteria; and the clinically meaningful response is induced in the subject in response to the administration.

26-27. (canceled)

28. The method of claim 1, wherein the subject at baseline has fibrosis.

29-31. (canceled)

32. The method of claim 1, wherein the myeloproliferative neoplasm is selected from myelofibrosis (MF), polycythemia vera (PV), primary or essential thrombocythemia (ET), and chronic myelomonocytic leukemia (CMML).

33. The method of claim 1, wherein the myeloproliferative neoplasm is a myelofibrosis (MF) selected from pre-fibrotic myelofibrosis (pre-PMF), primary or idiopathic MF (PMF), post-essential thrombocythemia MF (post-ET MF) and post-polycythemia vera MF (post-PV MF), optionally wherein the myeloproliferative neoplasm is Intermediate-1, Intermediate-2, or High Risk myelofibrosis on DIPSS scoring system.

34-35. (canceled)

36. The method of claim 1, wherein the subject is positive for a mutation selected from JAK2V617F, MPLW515L, CALR, and any combination thereof.

37. (canceled)

38. The method of claim 1, wherein the compound (1) or the pharmaceutically acceptable salt thereof is administered as monotherapy for the myeloproliferative neoplasm and the subject has previously been treated with a JAK inhibitor.

39. The method of claim 1, wherein the compound (1) or the pharmaceutically acceptable salt thereof is administered in combination with momelotinib or a pharmaceutically acceptable salt thereof, and
wherein the subject has previously been treated with a JAK inhibitor other than momelotinib or the pharmaceutically acceptable salt thereof.

40. The method of claim 1, wherein the compound (1) or the pharmaceutically acceptable salt thereof is administered in combination with ruxolitinib or a pharmaceutically acceptable salt thereof, and
wherein the subject is treatment naïve to a JAK inhibitor prior to initiating the method.

41-42. (canceled)

43. The method claim 1, comprising administering the compound (1) or the pharmaceutically acceptable salt thereof BID.

44. The method of claim 4, comprising administering to the subject the compound (1) or the pharmaceutically acceptable salt thereof and ruxolitinib or a pharmaceutically acceptable salt thereof or momelotinib or a pharmaceutically acceptable salt thereof in amounts that induce no overlapping hematological toxicities in terms of anemia and thrombocytopenia and transfusion dependence.

45. The method of claim 4, wherein the myeloproliferative neoplasm is selected from myelofibrosis (MF), polycythemia vera (PV), primary or essential thrombocythemia (ET), and chronic myelomonocytic leukemia (CMML).

46. The method of claim 4, wherein the compound (1) or the pharmaceutically acceptable salt thereof is administered as monotherapy for the myeloproliferative neoplasm and the subject has previously been treated with a JAK inhibitor.

47. The method of claim 4, wherein the compound (1) or the pharmaceutically acceptable salt thereof is administered in combination with momelotinib or a pharmaceutically acceptable salt thereof, and
wherein the subject has previously been treated with a JAK inhibitor other than momelotinib or the pharmaceutically acceptable salt thereof.

48. The method of claim 4, wherein the compound (1) or the pharmaceutically acceptable salt thereof is administered in combination with ruxolitinib or a pharmaceutically acceptable salt thereof, and
wherein the subject is treatment naïve to a JAK inhibitor prior to initiating the method.

49. A method of treating a myeloproliferative neoplasm by inducing:
(a) TSS50 in a human subject in need thereof;
(b) a probability of SVR20 in the human subject affected by the myeloproliferative neoplasm about ≥15%; or
(c) a combination of (a) and (b),
the method comprising administering to the subject a therapeutically effective amount of compound (1): 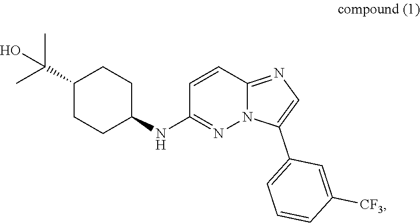 [SMILES: CC(C)(O)[C@H]1CC[C@H](Nc2ccc3ncc(-c4cccc(C(F)(F)F)c4)n3n2)CC1]
or a pharmaceutically acceptable salt thereof and optionally momelotinib or a pharmaceutically acceptable salt thereof,
wherein:
the myeloproliferative neoplasm is treated by inducing TSS50 in the subject, the treatment induces a probability of SVR20 in the subject about ≥15%, or a combination thereof, and
the subject has poor bone marrow functioning characterized by:
(i) thrombocytopenia;
(ii) anemia;
(iv) transfusion dependence; or
(v) a combination thereof.
